(19) United States
(12) Patent Application Publication
(10) Pub. No.: US 2025/0186489 A1
(43) Pub. Date: Jun. 12, 2025

(54) REPROGRAMMING OF CELLS TO TYPE 1 CONVENTIONAL DENDRITIC CELLS OR ANTIGEN-PRESENTING CELLS

(71) Applicant: Asgard Therapeutics AB, Lund (SE)

(72) Inventors: Fábio Fiúza Rosa, Leiria (PT); Olga Zimmermannova, Svetla Nad Sazavou (CZ); Alexandra Gabriela Barros Ferreira, Lomar-Braga (PT); Ervin Ascic, Vienna (AT); Cristiana Ferreira Pires, Palhaça (PT); Carlos Filipe Ribeiro Lemos Pereira, Caminha (PT)

(21) Appl. No.: 18/560,525

(22) PCT Filed: May 19, 2022
(86) PCT No.: PCT/EP2022/063606

§ 371 Date: Nov. 13, 2023

(30) Foreign Application Priority Data
May 19, 2021 (EP) 21174802.5
Feb. 23, 2022 (EP) 22158117.6

(51) Int. Cl. A61K 35/15; A61K 40/19; A61K 40/42; A61K 48/00; A61P 35/00; C07K 14/47; C07K 14/74; C12N 5/00; C12N 5/0784; C12N 15/86

(52) U.S. Cl. CPC A61K 35/15; A61K 40/19; A61K 40/4213; A61K 48/005; A61P 35/00; C07K 14/4702; C07K 14/70539; C12N 5/0018; C12N 5/0639; C12N 15/86; C12N 2501/73; C12N 2710/10043; C12N 2740/15043; C12N 2750/14143

(57) ABSTRACT

The present invention relates to compositions comprising transcription factors under control of promoter regions, wherein said compositions can be used for reprogramming cells to type 1 conventional dendritic cells or antigen-presenting cells. The invention further relates to methods for reprogramming cells into type 1 conventional dendritic cells or antigen-presenting cells.

### TECHNICAL FIELD

The present invention relates to compositions and methods for reprogramming cells to type 1 conventional dendritic cells or antigen-presenting cells.

### BACKGROUND

Cellular reprogramming relies on rewiring the epigenetic and transcriptional network of one cell state to that of a different cell type. Transcription factor (TF)-overexpression experiments have highlighted the plasticity of adult somatic or differentiated cells, providing new technologies to generate any desired cell type. Through forced expression of TFs, it is possible to reprogram somatic or differentiated cells into induced pluripotent stem cells (iPSCs) that are remarkably similar to embryonic stem cells (Takahashi et al., 2007; Takahashi & Yamanaka, 2006). Alternatively, a somatic cell can also be directly converted into another specialized cell type (Pereira, Lemischka, & Moore, 2012). Direct lineage conversion has proven successful to reprogram mouse and human fibroblasts into several cell types, such as neurons, cardiomyocytes and hepatocytes, using TFs specifying the target-cell identity (Xu, Du, & Deng, 2015). Direct cell conversions were also demonstrated in the hematopoietic system, where forced expression of TFs induced a macrophage fate in B cells and fibroblasts (Xie, Ye, Feng, & Graf, 2004) and the direct reprogramming of mouse fibroblasts into clonogenic hematopoietic progenitors was achieved with Gata2, Gfi1b, cFos and Etv6 (Pereira et al., 2013). These four TFs induce a dynamic, multi-stage hemogenic process that progresses through an endothelial-like intermediate, recapitulating developmental hematopoiesis in vitro (Pereira et al., 2016). Reprogrammed cells are very promising therapeutic tools for regenerative medicine, and cells obtained by differentiation of iPSCs are already being tested in clinical studies (Pires et al., 2019). Recently, it has been demonstrated that antigen-presenting dendritic cells (DCs) can be reprogrammed from unrelated cell-types by a small combination of TFs (Rosa et al., 2018), opening up the opportunity to apply cell reprogramming to modulate immune responses and develop new immunotherapies. Classically, the DC compartment can be divided in two functionally different DC subsets: conventional DCs (cDCs), which are professional antigen-presenting cells (APCs), and plasmacytoid DCs (pDCs). cDCs drive antigen-specific immune responses, while pDCs are professional producers of type I interferons during viral infection. However, the timing and exact mechanisms regulating the divergence of the different subsets during DC development are still to be established.

DCs are a class of bone-marrow-derived cells arising from lympho-myeloid hematopoiesis that scan the organism for pathogens, forming an essential interface between the innate immune system and the activation of adaptive immunity. DCs act as professional APCs capable of activating T cell responses by displaying peptide antigens complexed with the major histocompatibility complex (MHC) on the surface, together with all the necessary soluble and membrane associated co-stimulatory molecules. DCs induce primary immune responses by priming naïve T-lymphocytes, potentiate the effector functions of previously primed T-lymphocytes and orchestrate communication between innate and adaptive immunity. DCs are found in most tissues, where they continuously sample the antigenic environment and use several types of receptors to monitor for invading pathogens. In a steady state, and at an increased rate upon detection of pathogens, sentinel DCs in non-lymphoid tissues migrate to the lymphoid organs where they present to T cells the antigens they have collected and processed. The phenotype acquired by the T cell depends on the context of antigen presentation. If the antigen is derived from a pathogen, or damaged self, DCs will receive danger signals, becoming activated and subsequently stimulating T cells to become effectors, necessary to provide protective immunity.

An important aspect of the control of immune responses is the existence of different types of DCs, each specialized to respond to particular pathogens and to interact with specific subsets of T cells. In this context, cDCs can be further divided in myeloid/conventional DC type 1 (cDC1 or cDC1s) and myeloid/conventional DC type 2 (cDC2). This expands the flexibility of the immune system to react appropriately to a wide range of different pathogens and danger signals.

Human cDC1s, characterized by surface expression of CD141, CLEC9A, XCR1 and CD226 (Wculek et al., 2019; Heidkamp et al., 2016; Dutertre et al., 2019), are defined functionally by secreting immune-modulatory cytokines, including IL-12, and interferons (IFN), and chemokines such as CXCL10, and by cross-presenting antigens to CD8⁺ T cells (Lauterbach et al., 2010; Poulin et al., 2010). In the context of anti-tumor immunity, Batf3−/− animals lacking cDC1s fail to reject immunogenic tumours (Hildner et al., 2008). This effect was shown to depend on tumor-resident cDC1s, highlighting the importance of this DC subset at the tumor site to mediate immune-rejection of established tumours (Bottcher et al., 2018) and response to therapy (Salmon et al., 2016, Spranger et al., 2017). Accordingly, cDC1 abundance in human tumours was associated with patient survival and responsiveness to checkpoint inhibitors (Barry et al., 2018, Broz et al., 2014, Hubert et al., 2020, Mayoux et al. 2020, Spranger et al. 2017). Human primary cDC1s are very rare in vivo, so their study and translational applications require methods to generate functional cDC1s in vitro. Human CD34⁺ bone marrow (BM) progenitors have been used to derive CD141⁺cDC1s in vitro in the presence of FLT3L with SCF, GM-CSF and IL-4 (Poulin et al., 2010). More recently, FLT3L was combined with co-culture with Notch-expressing stromal cell lines to favour cDC1 differentiation (Kirkling et al., 2018; Balan et al., 2018). The generation of cDC1-, cDC2- and pDC-like cells from induced pluripotent stem cell (iPSC) cultures was also demonstrated (Sontag et al., 2017). However, these protocols are complex, require feeder layers and result in low yields as well as a mixture of different DC subsets with conflicting functions.

Thus, new strategies are necessary to generate homogeneous populations of differentiated human cDC1s in vitro.

### SUMMARY

Provided herein are compositions and methods for reprogramming cells into dendritic or antigen-presenting cells. The inventors have discovered that reprogramming of cells can be significantly improved by expression of transcription factors BATF3, IRF8, PU.1 under certain promoters. The inventors have also discovered additional transcription factors (i.e. IRF7 and BATF) that increase reprogramming efficiency when co-expressed with PU.1, IRF8 and BATF3.

Thus, provided herein is a composition comprising one or more constructs or vectors, which upon expression encodes the transcription factors:
a) BATF3, or a biologically active variant thereof, wherein the biologically active variant is at least 70% identical to SEQ ID NO: 10 (BATF3), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 10 (BATF3);
b) IRF8, or a biologically active variant thereof, wherein the biologically active variant is at least 70% identical to SEQ ID NO: 11 (IRF8), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 11 (IRF8); and
c) PU.1, or a biologically active variant thereof, wherein the biologically active variant is at least 70% identical to SEQ ID NO: 12 (PU.1), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 12 (PU.1);

wherein the one or more constructs or vectors comprise a promoter region capable of controlling the transcription of the transcription factors, wherein the promoter region comprises spleen focus-forming virus (SFFV) promoter.

Further provided herein is a cell comprising one or more constructs or vectors according to the compositions described herein.

Also provided herein is a method of reprogramming or inducing a cell into a dendritic or antigen-presenting cell, the method comprising the following steps:
a) transducing a cell with a composition comprising a construct or vector according to the compositions described herein.
b) expressing the transcription factors;

whereby a reprogrammed or induced cell is obtained.

Further provided herein is a reprogrammed or induced cell obtained according to the methods disclosed herein.

Also provided herein is a method of treating cancer, the method comprising administering to an individual in need thereof the composition; the cell; the pharmaceutical composition; and/or the reprogrammed or induced cell according to the present invention.

Further provided herein is the use of the composition; the cell; the pharmaceutical composition; and/or the reprogrammed or induced cell according to the present invention, for the manufacture of a medicament for the treatment of cancer.

### DESCRIPTION OF DRAWINGS

FIG. 1. PU.1, IRF8 and BATF3 induce global cDC1 gene expression program in human fibroblasts. (A) Human embryonic fibroblasts (HEF) were co-transduced with Dox-inducible lentiviral particles encoding PU.1, IRF8 and BATF3 (PIB, TetO-PIB) and M2rtTA (UbC-M2rtTA). Purified PIB-transduced HEFs (hiDC) were profiled by single-cell RNA-seq at day 3 (d3, CD45⁺), day 6 (d6, CD45⁺) and day 9 (CD45⁺ HLA-DR⁻, d9 DR⁻; CD45⁺ HLA-DR⁺, d9 DR⁺). HEF and peripheral blood cDC1, cDC2 and pDC were included as controls. (B) Flow cytometry analysis of hiDCs at day 3 and 9 after addition of Dox and (C) Kinetics of DR⁻ (top) and DR⁺ (bottom) cell emergence (n=2-8, mean±SD). (D) Scanning electron microscopy at day 9. Scale bars, 10 μm. (E) t-SNE plot showing 45,870 single cells. (F) Integration with published DC subset data (Villani et al. 2017) using scPred (Alquicira-Hernandez et al. 2019). Heat map shows the percentage of single cells affiliated to cDC1-DC6 subsets. (G) t-SNE plot of cDC1-affiliated single cells. (H) Violin plots showing gene expression distribution of cDC1-specific genes. Log values of gene counts are shown. (I) Heat map showing differentially expressed genes across profiled populations in 5 clusters. (J) Violin plots for genes selected from cluster 3. (K) Top five Reactome pathways enriched in each gene cluster. (L) Heat map and (M) violin plots showing expression of genes associated with antigen cross-presentation.

FIG. 2. Pseudo-temporal ordering of single cells highlights pathways associated with successful and unsuccessful cDC1 reprogramming. (A) Monocle 3 reconstruction of single-cell trajectories for HEF, hiDC at day 3, 6, and 9 (DR⁻ and DR⁺) unaffiliated or affiliated with cDC1 with scPred (Alquicira-Hernandez et al. 2019) and filtered cDC1. (B) cDC1 reprogramming trajectory colored by relative trajectory position (pseudotime, left). Whisker box plots showing pseudotime distribution by cell type (right). (C) tSNE plot showing single-cell velocities generated with scVelo (Bergen et al. 2020). Arrows indicate direction and thickness speed along trajectory. (D) Heat map highlighting 6 gene clusters (A-F) with dynamic expression along scVelo latent time. (E) Top 5 Reactome pathways enriched in each cluster. (F) Heat map showing mean expression values of gene modules by cell-type differentially expressed along trajectory. (G) Violin plots showing expression distribution of genes associated with unsuccessful and successful DC reprogramming. Log values of gene counts are shown. (H) Unsuccessful (left) and successful (right) cDC1 reprogramming transcription factorwith Chea3. SPI1, IRF8 and BATF are highlighted in bold. (I) Flow cytometry analysis of CD226 expression in hiDCs. (J) Classification with DC subset data (Villani et al. 2017). (K) Dead cell engulfment by CD45⁺HLA-DR⁺CD226⁺ and CD45⁺HLA-DR⁺CD226⁻ hiDC (n=6-7, mean±SD). (L) Reprogramming efficiency at day 9 generated by co-transduction of PIB with indicated transcription factors (n=4, mean±SD). M2rtTA- and PIE-transduced cells were included as controls. **p<0.005; ****p<0.00005.

FIG. 3. Inflammatory cytokine signalling enables human cDC1 reprogramming at high efficiency. (A) Quantification of hiDCs (CD45⁺HLA-DR⁺) at day 9 obtained in the presence of individual cytokines and (B) combinations of 2-3 cytokines. Non-transduced HEF were included as control (n=2-10, mean±SD).

FIG. 4. Enforced expression of transcription factors enable human cDC1 reprogramming at high efficiency. (A) Quantification of reprogrammed cells (tdT⁺ MHC-II⁺) obtained by transducing Clec9a-tdTomato (tdT) reporter mouse embryonic fibroblasts with PIB-IRES-GFP driven by Dox-inducible (TetO) or constitutive promoters (UbC, SFFV, PGK, EF1S, EF1 and EF1i). Expression of GFP (tetO-GFP) was used as control (n=2-6, mean±SD). (B) Quantification of hiDCs at day 9 generated with TetO-PIB or SFFV-PIB, in the presence or absence of IFN-γ, IFN-β and TNF-α (n=4-19, mean±SD). (C) hiDC yield per input fibroblast (n=10-12, mean±SD). (D) hiDCs at day 9 generated in the four conditions were purified and profiled by scRNA-seq. Heat map shows the percentage of single cells affiliated to cDC1-DC6 subsets.

FIG. 5. Anti-inflammatory cytokine signalling does not impair cDC1 reprogramming. Flow cytometry quantification of CD45⁺HLA-DR⁺ cells (left) and CD40⁺ cells gated in CD45⁺HLA-DR⁺ cells (right) at day 9 generated by transducing HEF with SFFV-PIB in the presence of anti-inflammatory cytokines (n=3, mean±SD).

FIG. 6. Optimized reprogramming protocol allows generation of functional human cDC1-like cells. (A) Median fluorescence intensity (MFI) of CD40 and CD80 in hiDCs (CD45⁺HLA-DR⁺) at day 9 generated with SFFV-PIB in the absence or presence of IFN-γ, IFN-β and TNF-α (hiDC+cyt) and peripheral blood CD141⁺CLEC9A⁺cDC1. Cells were stimulated overnight with individual TLR agonists LPS, Poly I:C (Polyinosinic:polycytidylic acid), R848 or combination (All) (n=2-14, mean±SD). (B) Quantification of dead cell engulfment by hiDC at day 9 after 2-hour incubation. HEF and CD141⁺CLEC9A⁺cDC1 were included as controls (n=3-12, mean±SD). (C) Cytokine secretion of purified hiDC at day 9 after overnight incubation with TLR agonists. HEF, monocyte-derived DCs (moDC) and CD141⁺CLEC9A+XCR1⁺cDC1 were included as controls (n=2-11, mean±SD). (D) Cells were incubated overnight with LPS, Poly I:C and R848, pulsed with CMV protein for 3 hours, washed and co-cultured with CMV⁺ CD8⁺ T cells. Antigen cross-presentation was quantified by measuring IFN-γ after 24 h (n=2-4, mean±SD). *p<0.05; **p<0.005; ***p<0.0005; ****p<0.00005.

FIG. 7. Efficient cDC1 reprogramming of adult fibroblasts. (A) Flow cytometry analysis and (B) quantification of hiDC (CD45⁺ HLA-DR⁺) at day 9 generated from human dermal fibroblasts (HDF) in the absence (SFFV-PIB) or presence of IFN-γ, IFN-β and TNF-α (SFFV-PIB+cyt) from three independent donors. HDF were included as controls (n=3-13, mean±SD). (C) Expression of CD40 and CD80. (D) HDF-derived hiDC at day 9 were purified and profiled by scRNA-seq. Heat map shows percentage of single cells affiliated to cDC1-6 subsets. (E) Heatmap showing expression of genes upregulated during reprogramming and expressed in cDC1s. cDC1 and antigen presentation genes are highlighted in bold and shown in (F) as violin plots. Log values of gene counts are shown.

FIG. 8. Efficient cDC1 reprogramming of mesenchymal stromal cells. (A) Strategy to derive hiDC from human Mesenchymal Stromal Cells (MSC) under xeno-free conditions. MSC were isolated from three healthy donors, FACS-purified (Lin-CD45-CD271⁺), expanded in pHPL media, transduced and cultured in X-VIVO 15. dX=Day X. (B and C) Quantification of MSC-derived hiDCs at day 9 generated with or without cytokines (n=3-14, mean±SD). (D) Flow cytometry analysis of CD40 and CD80. ns—non significant; ****p<0.00005.

FIG. 9. Induced DCs elicit anti-tumor immunity in vivo. (A) Kinetics of acquisition of cross-presentation ability during DC reprogramming. Representative flow cytometry plots showing CTV labelling of TCR⁺CD8⁺CD44⁺ T cells co-cultured with 50,000 tdT⁺ cells at d4, d7 and d9 of reprogramming. MEF were included as control. (B) Quantification of proliferative T cells after co-culture with tdT⁺ cells sorted at different time points and in three different ratios (n=4, mean±SD). (C) Cytokine secretion of sorted tdT⁺ cells after stimulation with LPS or Poly I:C (n=2, mean±SD). MEF and CD103⁺ bone-marrow-derived DCs (BM-DCs) were included as control. (D) Purified tdT⁺ iDCs were mixed with 0.5M B16OVA cells before subcutaneous implantation of the tumours in C57BL/6 mice. Tumor volume was evaluated during 14 days (n=5-6, mean±SEM). (E) Purified tdT⁺ iDCs were intra-tumorally injected in B16OVA tumours 8 days after establishment. Tumor volume was evaluated until day 20 (n=4-9, mean±SEM, 2 independent experiments). PBS, MEF and CD103⁺ BM-DC-injected animals were included as controls. (F) CTV-labeled OT-I CD8⁺ T-cells were injected intravenously at the same day as iDCs and tumours and tumor-draining lymph nodes analyzed after 4 days. Tumor infiltration (left) and IFN-γ and Granzyme B (GzmB) expression after in vitro re-stimulation (right) of OVA-restricted CD8⁺ T-cells were quantified (n=2-4, mean±SD).

FIG. 10. PU.1 has independent chromatin targeting capacity and recruits IRF8 and BATF3 to the same binding sites. (A) Strategy to profile chromatin binding sites of PU.1, IRF8 and BATF3 (PIB) at early stages of reprogramming. HDFs were transduced with PIB (left) or individual factors (right) and analyzed by ChIP-seq after 48 hours. (B) Heat maps showing genome-wide distribution of PU.1, IRF8 and BATF3 when expressed in combination (left) or individually (right). Signal is displayed within an 8 kb window centred on individual peaks. The number of peaks in each condition is shown. Average signal intensity of peaks is depicted (bottom). (C) De novo motif prediction analysis for PU1, IRF8 and BATF3 target sites when expressed in combination or individually. Motifs for PU.1 are highlighted in bold.

FIG. 11. PU.1, IRF8 and BATF3 bind at open chromatin to inhibit fibroblast genes and impose a cDC1 transcriptional program. (A) Venn diagram shows genome-wide peak overlap between PU.1, IRF8 and BATF3 (PIB). (B) De novo motif prediction analysis for PU.1, IRF8 and BATF3 co-bound sites when expressed in combination. Motifs for PU.1-IRF and BATF are highlighted in bold. (C) Motif comparison between PU.1-IRF and BATF. Jaccard similarity coefficient=0.02. (D) Immunoblots showing immunoprecipitation (IP) for PU.1 (top), IRF8 (middle), and BATF3 (bottom) in HEK293T cells 24 hours after transfection with PIB (left). Co-immunoprecipitation (Co-IP) performed with 1, 2 and 5 million (M) cells (right). Input (10%) and IgG isotype were used as controls. (E) Heat map showing differentially expressed genes between HDFs and hiDC at day 9 that are bound by either PU.1, IRF8 and BATF3 or the three factors (intersect). (F) Heat maps of normalized read coverages of chromatin marks in HDFs for co-bound sites. Signal is displayed within an 8 kb window and centered on transcription factor binding sites. Average signal intensity is shown (upper panel). (G) Model for the mechanism to set in motion cDC1 reprogramming.

FIG. 12. PU.1, IRF8 and BATF3 reprogram mouse cancer cells in cDC1-like cells. (A) Flow cytometry analysis of mouse lewis lung carcinoma (3LL) and melanoma (B16) cells at day 9 after transduction with SFFV-PIB-GFP lentiviral particles (tumour-antigen presenting cells, Tumour-APCs). SFFV-GFP transduced parental cell lines were included as controls. (B) Lewis Lung carcinoma (LLC) and melanoma B16-derived reprogrammed 2 cells (GFP⁺CD45⁺ MHC-II⁺) were purified by FACS at day 9 (d9). Cancer cells 3 transduced with GFP vector were included as controls (d0). Heatmaps show expression 4 genes related to IFN-γ (left) and STING (right) pathways in reprogrammed LLC and induced 5 dendritic cells (iDCs). Splenic dendritic cells type 1 (cDC1) were included as reference 6 (GSE103618). (C) Flow cytometry analysis (left) and quantification (right) of endogenous antigen presentation measured as CD8⁺ T cell proliferation (CTV dilution) and activation (CD44⁺) after co-culture with FACS-purified B16-OVA cells transduced with PIB or eGFP lentiviruses at reprogramming day 3 after overnight stimulation with Poly (I:C) (P(I:C)), when indicated (n=3-4). (D) Flow cytometry analysis (left) and quantification (right) of T cell mediated killing of B16-OVA target cells (mOrange⁺) that were either PIB-transduced or IFN-treated, after 72 h of co-culture (n=6-9). (E) Flow cytometry quantification of antigen cross-presentation capacity measured as percentage of CD44⁺ proliferative OT-I CD8⁺9 T cells after co-coculture with B16 cells transduced with SFFV-PIB-GFP lentiviral particles and incubated overnight with OVA protein in the presence or P(I:C) and/or Interferon gamma (IFN-g), when indicated (n=4-8). (F) B16 derived tumour-APCs at reprogramming day 5 were pulsed with OVA protein and P(I:C) and injected intratumorally at day 7, 10 and 13 in pre-established B16-OVA tumours. (G) Tumour growth and (H) survival in mice injected with tumour-APCs (PIB), PBS or B16 cells transduced with control lentiviruses (MCS) (n=6). Mean±SD is represented. **p<0.01, ****p<0.0001.

FIG. 13. PU.1, IRF8 and BATF3 reprogram human cancer cells into cDC1-like cells. (A) Reprogramming efficiency of glioblastoma (T98G), rectal carcinoma (ECC4) and mesothelioma (ACC-Meso-1, ACCM1) cell lines, analyzed by flow cytometry as the percentage of cells co-expressing CD45 and HLA-DR gated in transduced EGFP⁺ cells (red), when transduced with SFFV-PIB-GFP or control SFFV-GFP lentiviruses. (B) cDC1-reprogramming efficiency across 28 solid tumour cell lines. Reprogrammed populations expressing (CD45⁺HLA-DR⁺) and intermediate populations (CD45⁺HLA-DR⁻ or CD45-HLA-DR⁺) are shown (n=2-8). Mean±SD represented. (C) Flow cytometry quantification of cDC1 surface markers (CLEC9A, CD141, CD11c) in human glioblastoma (T98G) cells 9 days after transduction with SFFV-PIB-GFP lentiviral particles. SFFV-GFP transduced parental cell lines were included as controls. (D) Kinetics of surface expression of the co-stimulatory molecules CD40, CD80 and CD86 in CD45⁺ HLA-DR⁺ cancer cells 9 days after transduction with SFFV-PIB-GFP lentiviral particles stimulated overnight with Poly I:C and LPS. SFFV-GFP transduced parental cell lines and un-stimulated SFFV-PIB-GFP transduced parental cell lines were included as controls. Exemplificative plots at day 9 are shown in (E). (F) Human primary tonsil carcinoma tissue (JCA10) and patient-xenograft derived bladder carcinoma cells (U3P2E2) were transduced with hPIB-IRES-EGFP or EGFP control vector (day 0) and analysed by flow cytometry at day 9 to determine the percentage of reprogrammed CD45⁺ HLA-DR⁺ cells (black) and partially reprogrammed cells expressing either CD45 or HLA-DR. (G) Reprogramming efficiency shown in primary human tumour cells from melanoma (n=2), lung (n=2), head and neck (tonsil, n=2; tongue, n=3), pancreatic (n=2), breast (n=2), and bladder carcinoma (n=2) as well as cancer-associated fibroblasts (CAFs, n=2). Partially reprogrammed cells are shown (CD45⁺ HLA-DR⁻, CD45-HLA-DR⁺).

FIG. 14. PU.1, IRF8 and BATF3 induce rapid global transcriptional and epigenetic reprogramming. (A) Experimental design to evaluate the kinetics of transcriptomic and epigenetic reprogramming. The human glioblastoma cell line (T98G) was transduced with SFFV-hPIB-IRES-EGFP. Reprogrammed (CD45⁺ HLA-DR⁺, ++) and partially reprogrammed (CD45-HLA-DR⁺, +) cells were FACS sorted and profiled with mRNA-sequencing and ATAC-sequencing at day 3 (d3), 5 (d5), 7 (d7) and 9 (d9). Control cells transduced with empty EGFP vector are represented as day 0 (d0). cDC1 donor cells were used as reference. (B) Principal component analysis (PCA) of cancer cell reprogramming time-course based on differentially expressed genes (left panel). Reprogramming of human embryonic fibroblasts (HEF) was also included as a reference for the dynamics of the process. Arrow highlights reprogramming trajectories. PCA based on differentially accessible chromatin regions (right panel). Donor peripheral blood cDC1 were used as reference. (C) Establishment of tumour-APC transcriptomic signature in reprogrammed and partially reprogrammed T98G cells (left). Chromatin accessibility at the tumour-APC gene set is shown on the right.

FIG. 15. Histone deacetylase inhibition enhances tumour-APC reprogramming efficiency. (A) Lewis Lung Carcinoma (LLC) and B16 cancer cells were transduced with PU.1, IRF8 and BATF3 (SFFV-PIB-eGFP), cultured in the presence or absence of valproic acid (VPA) and analysed by flow cytometry at day 9 for CD45 and MHC-II expression. (B) Quantification of reprogramming efficiency (% CD45⁺ MHC-II⁺ cells) in the presence of VPA, gated in eGFP⁺ transduced cells (n=6-16). Cancer cells transduced with eGFP vector (green, striped) were included as control. (C) Quantification of the percentage of MHC-I⁺ cells gated in eGFP⁺transduced cells (n=6-11). (D) Quantification of CD44⁺ proliferative OT-I CD8⁺ T cells after co-culture with reprogrammed LLC-OVA and B16-OVA cells (n=4-11). (E) Quantification of T cell mediated killing by flow cytometry of reprogrammed B16-OVA target cells (mOrange⁺), and co-cultured with non-target B16-OVA cells after 0 and 72 h in a 1:1 ratio of activated OT-I T cells and cancer cells (n=5-7). (F) Quantification of CD44⁺ proliferative OT-I CD8⁺ T cells after co-culture with reprogrammed LLC or B16 cells pre-incubated with OVA peptides (SIINFEKL) (n=4-12). Mean±SD is represented. **p<0.01, ****p<0.0001. (G) Flow cytometry analysis (top panel) and quantification (lower panel) of cDC1 reprogramming efficiency in the presence and absence of valproic acid (VPA) in 6 human cancer cell lines at day 9. Cancer cell lines were transduced with SFFV-hPIB-IRES-EGFP lentiviral particles or EGFP as control and cultured in the presence or absence of VPA from day 1 to day 4 of reprogramming.

FIG. 16. SPIB and SPIC compensate PU.1 role in cDC1 reprogramming. (A) Flow cytometry quantification of Clec9a reporter activation in mouse embryonic fibroblasts (MEFs) 5 days after transduction with PU.1 homologs alone or in combination with IRF8 and BATF3. (B) Flow cytometry quantification of CD45 and MHC-II expression levels (gated in tdTomato⁺ cells). Graph bars indicate the mean±SEM (N=4). **p<0.01, ****p<0.0001.

FIG. 17. PU.1, IRF8 and BATF3 delivered by Adenovirus and Adeno-associated virus allows cDC1 reprograming in mouse and human cells. (A) Flow cytometry analysis of Clec9a reporter activation and (B) quantification of CD45 and MHC-II expression in mouse embryonic fibroblasts (MEFs) 9 days after transduction with lentivirus (Lenti), Adenovirus (Ad5 and Ad5/F35) and Adeno-associated virus (AAV-DJ and AAV2-qYF) encoding PU1, IRF8 and BATF3 (PIB) and GFP (PIB-GFP). Virus encoding GFP only were included as controls, and did not induce tdTomato expression in transduced cells. Graph bars indicate the mean±SEM (N=4). (C) Flow cytometry quantification of cDC1 reprogramming efficiency in B2905 mouse melanoma cell line measured as expression of CD45 and MHC-II 9 days after transduction with virus encoding PIB-GFP. Lentivirus encoding GFP only were included as controls. Graph bars indicate the mean±SEM (N=4). (D) Flow cytometry quantification of cDC1 reprogramming efficiency in 2 human cancer cell lines (IGR-39 and T98G) and 1 primary melanoma sample (2778) measured as expression of CD45 and HLA-DR 9 days after transduction with virus encoding PIB-GFP. Lentivirus encoding GFP only were included as controls. Graph bars indicate the mean±SEM (N=4).

### DETAILED DESCRIPTION

### Definitions

“Biologically active variant” refers herein to a biologically active variant of a transcription factor (TF), which retains at least some of the activity of the parent TF. For example, a biologically active variant of Basic Leucine Zipper ATF-Like Transcription Factor 3 (BATF3), Interferon Regulatory Factor 8 (IRF8), and PU.1 can act as said respective TF and induce or inhibit expression of the same genes in a cell as BATF3, IRF8, and PU.1, respectively, do, although the efficiency of the induction may be different, e.g. the efficiency of inducing or inhibiting genes is decreased or increased compared to the parent TF.

“Identity and homology”, with respect to a polynucleotide or polypeptide, are defined herein as the percentage of nucleic acids or amino acids in the candidate sequence that are identical or homologous, respectively, to the residues of a corresponding native nucleic acids or amino acids, after aligning the sequences and introducing gaps, if necessary, to achieve the maximum percent identity/similarity/homology, and considering any conservative substitutions according to the NCIUB rules (hftp://www.chem.qmul.ac.uk/iubmb/misc/naseq.html; NC-IUB, Eur J Biochem (1985)) as part of the sequence identity. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given homology between two sequences implies that the identity between these sequences is at least equal to the homology; for example, if two sequences are 70% homologous to one another, they cannot be less than 70% identical to one another—but could be sharing 80% identity.

“Mesenchymal stem cells” or “Mesenchymal stromal cells” (both referred to as MSCs”) are used herein interchangeably, and are referred to herein as being multipotent stromal cells that can differentiate into a variety of cell types, including but not limited to: osteoblasts (bone cells), chondrocytes (cartilage cells), and adipocytes (fat cells).

“Murine” refers herein to any and all members of the family Muridae, including rats and mice.

“Reprogramming” refers herein to the process of converting of differentiating cells from one cell type into another. In particular, reprogramming herein refers to converting or transdifferentiating any type of cell into a type 1 conventional dendritic cell or an antigen-presenting cell.

“Treating,” or “Treatment,” refers herein to any administration or application of a therapeutic for the disclosed diseases, disorders and conditions in subject, and includes inhibiting the progression of the disease, slowing the disease or its progression, arresting its development, partially or fully relieving the disease, or partially or fully relieving one or more symptoms of a disease.

As used herein, the term “adenovirus” is used to refer to any and all viruses that may be categorized as an adenovirus, including any adenovirus that infects a human or a non-human animal, including all groups, subgroups, and serotypes, except when required otherwise. Thus, as used herein, “adenovirus” refers to the virus itself or derivatives thereof and cover all serotypes and subtypes, naturally occurring (wild-type), modifications to be used as an adenoviral vector, e.g., a gene delivery vehicle, forms modified in ways known in the art, such as for example capsid mutations, and recombinant forms, replication-competent, conditionally replication-competent, or replication-deficient forms, except where indicated otherwise.

As used herein, the term “adeno-associated virus” may be used to refer to the naturally occurring wild-type virus itself or derivatives thereof. The term is used to refer to any and all viruses that may be categorized as an adeno-associated virus, including any adeno-associated virus that infects a human or a non-human animal, and covers all subtypes, serotypes and pseudotypes, and both naturally occurring, modified and recombinant forms, such as modifications to be used as an adeno-associated viral vector, e.g., a gene delivery vehicle except where required otherwise.

As used herein, the abbreviation “Ad” in the context of a viral vector refers to an adenovirus and is typically followed by a number indicating the serotype of the adenovirus. For example, “Ad5” refers to adenovirus serotype 5.

Any Ad suitable for the purpose may be used herein, such as but not limited to Ad from any serotype from any of the A, B, C, D, E, F, G Ad subgroups, for example Ad2, Ad5, or Ad35, avian Ad, bovine Ad, canine Ad, caprine Ad, equine Ad, primate Ad, non-primate Ad, and ovine Ad. “Primate Ad refers to Ad that infect primates, “non-primate Ad” refers to Ad that infect non-primate mammals, “bovine Ad refers to Ad that infect bovine mammals.

The genomic sequences of various serotypes of Ad, as well as the sequences of the native terminal repeats (TRs) and capsid subunits are known in the art.

As used herein, the abbreviation “AAV” in the context of a viral vector refers to an adeno-associated virus and is typically followed by a number indicating the serotype of the adeno-associated virus. For example, “AAV2” refers to adeno-associated virus serotype 2.

Any AAV suitable for the purpose may be used herein, such as but not limited to AAV serotype 1 (AAV1), AAV serotype 2 (AAV2), AAV serotype 3A (AAV3A), AAV serotype 3B (AAV3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), avian AAV, bovine AAV, canine AAV, caprine AAV, equine AAV, primate AAV, non-primate AAV, and ovine AAV. “Primate AAV refers to AAV that infect primates, “non-primate AAV” refers to AAV that infect non-primate mammals, “bovine AAV refers to AAV that infect bovine mammals.

The genomic sequences of various serotypes of AAV, as well as the sequences of the native terminal repeats (TRs), Rep proteins, and capsid subunits are known in the art.

“Hybrid” Ad or AAV vectors as used herein refers to vectors based on Ads or AAVs engineered in a way that the Ad or AAV vectors contains proteins derived from two or more different Ad or AAV serotypes.

“AAV2-qYF” or “AAV2-QuadYF” as used herein refers to a quadruple tyrosine to phenylalanine mutant of AAV2.

“AAV-DJ” as used herein refers to a hybrid capsid derived from DNA family shuffling of 8 wild type serotypes of AAV, including AAV2, 4, 5, 8, 9, avian, bovine and caprine AAV. AAV-DJ is a synthetic serotype, type 2/type 8/type 9 chimera, distinguished from its closest natural relative (AAV-2) by 60 capsid amino acids.

### Compositions

The present invention relates to compositions and their use in methods for reprogramming or inducing cells into dendritic or antigen-presenting cells. The inventors have surprisingly discovered that reprogramming can be significantly improved by expressing TFs BATF3, IRF8, and PU.1 under specific promoters.

Thus, provided herein is a composition comprising one or more constructs or vectors, which upon expression encodes the transcription factors:
a) BATF3, or a biologically active variant thereof, wherein the biologically active variant is at least 70% identical to SEQ ID NO: 10 (BATF3);
b) IRF8, or a biologically active variant thereof, wherein the biologically active variant is at least 70% identical to SEQ ID NO: 11 (IRF8); and
c) PU.1, or a biologically active variant thereof, wherein the biologically active variant is at least 70% identical to SEQ ID NO: 12 (PU.1);

wherein the one or more constructs or vectors comprise a promoter region capable of controlling the transcription of the transcription factors, wherein the promoter region comprises spleen focus-forming virus (SFFV) promoter, MND (myeloproliferative sarcoma virus enhancer, negative control region deleted, dl587rev primer-binding site substituted) promoter, CAG (CMV early enhancer/chicken β actin) promoter, cytomegalovirus (CMV) promoter, ubiquitin C (UbC) promoter, EF-1 alpha (EF-1α) promoter, EF-1 alpha short (EF1S) promoter, EF-1 alpha with intron (EF1i) promoter, phosphoglycerate kinase (PGK) promoter, or a promoter exhibiting essentially the same effect.

The TFs may be as defined herein in the section “Transcription factors”.

The promoter region may be as defined herein in the section “Promoters”.

The TFs may be expressed from one or more vectors or constructs as polycistronic constructs, dicistronic (or bicistronic) constructs, and/or monocistronic constructs. An mRNA molecule is said to be monocistronic when it contains the genetic information to translate only a single protein chain. On the other hand, polycistronic mRNA carries several open reading frames (ORFs), each of which is translated into a polypeptide. Dicistronic mRNA encodes only two proteins. Polycistronic and dicistronic mRNA are expressed from a single promoter or promoter region.

In one embodiment, the composition further comprises one or more constructs or vectors, which upon expression encode one or more transcription factors selected from:
a) IRF7, or a biologically active variant thereof, wherein the biologically active variant is at least 70% identical to SEQ ID NO: 21 (IRF7);
b) BATF, or a biologically active variant thereof, wherein the biologically active variant is at least 70% identical to SEQ ID NO: 19 (BATF);
c) SPIB, or a biologically active variant thereof, wherein the biologically active variant is at least 70% identical to SEQ ID NO: 23 (SPIB);
d) SPIC, or a biologically active variant thereof, wherein the biologically active variant is at least 70% identical to SEQ ID NO: 25 (SPIC);
e) CEBPα, or a biologically active variant thereof, wherein the biologically active variant is at least 70% identical to SEQ ID NO: 13 (CEBPα);

wherein the one or more constructs or vectors comprise a promoter region capable of controlling the transcription of the transcription factors, wherein the promoter region comprises spleen focus-forming virus (SFFV) promoter, MND (myeloproliferative sarcoma virus enhancer, negative control region deleted, dl587rev primer-binding site substituted) promoter, CAG (CMV early enhancer/chicken β actin) promoter, cytomegalovirus (CMV) promoter, ubiquitin C (UbC) promoter, EF-1 alpha (EF-1α) promoter, EF-1 alpha short (EF1S) promoter, EF-1 alpha with intron (EF1i) promoter, phosphoglycerate kinase (PGK) promoter, or a promoter exhibiting essentially the same effect.

In one embodiment, the composition comprises:
a) one construct or vector which upon expression encodes the transcription factors BATF3, IRF8 and PU.1;
b) one construct or vector which upon expression encodes the transcription factors BATF3, IRF8 and SPIB;
c) a first construct or vector which upon expression encodes the transcription factors BATF3 and IRF8, and a second construct or vector, which upon expression encodes the transcription factor PU1;
d) a first construct or vector which upon expression encodes the transcription factors BATF3 and IRF8, and a second construct or vector, which upon expression encodes the transcription factor SPIB;
e) a first construct or vector which upon expression encodes the transcription factor BATF3, and a second construct or vector, which upon expression encodes the transcription factors IRF8 and PU1;
f) a first construct or vector which upon expression encodes the transcription factor BATF3, and a second construct or vector, which upon expression encodes the transcription factors IRF8 and SPIB;
g) a first construct or vector which upon expression encodes the transcription factor IRF8, and a second construct or vector, which upon expression encodes the transcription factors BATF3 and PU.1;
h) a first construct or vector which upon expression encodes the transcription factor IRF8, and a second construct or vector, which upon expression encodes the transcription factors BATF3 and SPIB;
i) a first construct or vector which upon expression encodes the transcription factor BATF3, a second construct or vector which upon expression encodes the transcription factor IRF8, and a third construct or vector which upon expression encodes the transcription factor PU.1;

and/or
j) a first construct or vector which upon expression encodes the transcription factor BATF3, a second construct or vector which upon expression encodes the transcription factor IRF8, and a third construct or vector which upon expression encodes the transcription factor SPIB.

In one embodiment, the one or more constructs or vectors upon expression further encodes the transcription factor CCAAT/enhancer-binding protein alpha (cEBPa), or a biologically active variant thereof. cEBPa may be as defined herein in the section “Transcription factors”.

In another embodiment, the one or more constructs or vectors upon expression further encodes the transcription factor Interferon regulatory factor 7 (IRF7), or a biologically active variant thereof. IRF7 may be as defined herein in the section “Transcription factors”.

In another embodiment, the one or more constructs or vectors upon expression further encodes the transcription factor Basic Leucine Zipper ATF-Like (BATF), or a biologically active variant thereof. BATF may be as defined herein in the section “Transcription factors”.

In another embodiment, the one or more constructs or vectors upon expression further encodes the transcription factor Spi-C (SPIC), or a biologically active variant thereof. SPIC may be as defined herein in the section “Transcription factors”.

In one embodiment, the composition comprises:
a) one construct or vector which upon expression encodes the transcription factors BATF3, IRF8, PU.1 and IRF7;
b) a first construct or vector which upon expression encodes the transcription factors BATF3 and IRF8, and a second construct or vector which upon expression encodes the transcription factors PU.1 and IRF7;
c) a first construct or vector which upon expression encodes the transcription factors BATF3 and PU.1, and a second construct or vector which upon expression encodes the transcription factors IRF8 and IRF7;
d) a first construct or vector which upon expression encodes the transcription factors PU.1 and IRF8, and a second construct or vector which upon expression encodes the transcription factors BATF3 and IRF7;
e) a first construct or vector which upon expression encodes the transcription factors BATF3, IRF8 and PU.1, and a second construct or vector which upon expression encodes the transcription factor IRF7;
f) a first construct or vector which upon expression encodes the transcription factor BATF3, and a second construct or vector which upon expression encodes the transcription factors IRF8, PU.1 and IRF7;
g) a first construct or vector which upon expression encodes the transcription factor IRF8, and a second construct or vector which upon expression encodes the transcription factors BATF3, PU.1 and IRF7;
h) a first construct or vector which upon expression encodes the transcription factor PU.1, and a second construct or vector which upon expression encodes the transcription factors BATF3, IRF8 and IRF7; and/or
i) a first construct or vector which upon expression encodes the transcription factor BATF3; a second construct or vector which upon expression encodes the transcription factor IRF8; a third construct or vector which upon expression encodes the transcription factor PU.1 and a fourth construct or vector which upon expression encodes the transcription factor IRF7;

In one embodiment, the composition comprises:
a) one construct or vector which upon expression encodes the transcription factors BATF3, IRF8, PU.1 and BATF;
b) a first construct or vector which upon expression encodes the transcription factors BATF3 and IRF8, and a second construct or vector which upon expression encodes the transcription factors PU.1 and BATF;
c) a first construct or vector which upon expression encodes the transcription factors BATF3 and PU.1, and a second construct or vector which upon expression encodes the transcription factors IRF8 and BATF;
d) a first construct or vector which upon expression encodes the transcription factors PU.1 and IRF8, and a second construct or vector which upon expression encodes the transcription factors BATF3 and BATF;
e) a first construct or vector which upon expression encodes the transcription factors BATF3, IRF8 and PU.1, and a second construct or vector which upon expression encodes the transcription factor BATF;
f) a first construct or vector which upon expression encodes the transcription factor BATF3, and a second construct or vector which upon expression encodes the transcription factors IRF8, PU.1 and BATF;
g) a first construct or vector which upon expression encodes the transcription factor IRF8, and a second construct or vector which upon expression encodes the transcription factors BATF3, PU.1 and BATF;
h) a first construct or vector which upon expression encodes the transcription factor PU.1, and a second construct or vector which upon expression encodes the transcription factors BATF3, IRF8 and BATF; and/or
i) a first construct or vector which upon expression encodes the transcription factor BATF3; a second construct or vector which upon expression encodes the transcription factor IRF8; a third construct or vector which upon expression encodes the transcription factor PU.1 and a fourth construct or vector which upon expression encodes the transcription factor BATF;

In one embodiment, the composition comprises:
a) one construct or vector which upon expression encodes the transcription factors BATF3, IRF8, SPIB and IRF7;
b) a first construct or vector which upon expression encodes the transcription factors BATF3 and IRF8, and a second construct or vector which upon expression encodes the transcription factors SPIB and IRF7;
c) a first construct or vector which upon expression encodes the transcription factors BATF3 and SPIB, and a second construct or vector which upon expression encodes the transcription factors IRF8 and IRF7;
d) a first construct or vector which upon expression encodes the transcription factors SPIB and IRF8, and a second construct or vector which upon expression encodes the transcription factors BATF3 and IRF7;
e) a first construct or vector which upon expression encodes the transcription factors BATF3, IRF8 and SPIB, and a second construct or vector which upon expression encodes the transcription factor IRF7;
f) a first construct or vector which upon expression encodes the transcription factor BATF3, and a second construct or vector which upon expression encodes the transcription factors IRF8, SPIB and IRF7;
g) a first construct or vector which upon expression encodes the transcription factor IRF8, and a second construct or vector which upon expression encodes the transcription factors BATF3, SPIB and IRF7;
h) a first construct or vector which upon expression encodes the transcription factor SPIB, and a second construct or vector which upon expression encodes the transcription factors BATF3, IRF8 and IRF7;
and/or
i) a first construct or vector which upon expression encodes the transcription factor BATF3; a second construct or vector which upon expression encodes the transcription factor IRF8; a third construct or vector which upon expression encodes the transcription factor SPIB and a fourth construct or vector which upon expression encodes the transcription factor IRF7;

In one embodiment, the composition comprises:
a) one construct or vector which upon expression encodes the transcription factors BATF3, IRF8, SPIB and BATF;
b) a first construct or vector which upon expression encodes the transcription factors BATF3 and IRF8, and a second construct or vector which upon expression encodes the transcription factors SPIB and BATF;
c) a first construct or vector which upon expression encodes the transcription factors BATF3 and SPIB, and a second construct or vector which upon expression encodes the transcription factors IRF8 and BATF;
d) a first construct or vector which upon expression encodes the transcription factors SPIB and IRF8, and a second construct or vector which upon expression encodes the transcription factors BATF3 and BATF;
e) a first construct or vector which upon expression encodes the transcription factors BATF3, IRF8 and SPIB, and a second construct or vector which upon expression encodes the transcription factor BATF;
f) a first construct or vector which upon expression encodes the transcription factor BATF3, and a second construct or vector which upon expression encodes the transcription factors IRF8, SPIB and BATF;
g) a first construct or vector which upon expression encodes the transcription factor IRF8, and a second construct or vector which upon expression encodes the transcription factors BATF3, SPIB and BATF;
h) a first construct or vector which upon expression encodes the transcription factor SPIB, and a second construct or vector which upon expression encodes the transcription factors BATF3, IRF8 and BATF; and/or
i) a first construct or vector which upon expression encodes the transcription factor BATF3; a second construct or vector which upon expression encodes the transcription factor IRF8; a third construct or vector which upon expression encodes the transcription factor SPIB and a fourth construct or vector which upon expression encodes the transcription factor BATF;

The one or more constructs and vectors disclosed herein may be any type of constructs and vectors, such as a plasmid.

In one embodiment, the one or more constructs or vectors are one or more viral vectors. In other embodiments, the viral vector is selected from the group consisting of: lentiviral vectors, retrovirus vectors, adenovirus vectors, herpes virus vectors, pox virus vectors, adeno-associated virus vectors, paramyxoviridae vectors, rabdoviral vectors, alphaviral vectors, flaviral vectors, and adeno-associated viral vectors. In another embodiment, the viral vector is a lentiviral vector.

The adenovirus (Ad) and adeno-associated virus (AAV) vectors may be vectors derived from any Ad or AAV serotype known in the art and may permit gene expression in particular cells (e.g., nerve cells, muscle cells, and hepatic cells), tissues, and organs for instance by the application of the specificity of the target cells to be infected for each serotype. The Ad or AAV may be wild-type or have one or more of the wild-type genes deleted in whole or part. The Ad or AAV may be further engineered by any method known in the art, such as for example pseudotyping, resulting in hybrid (or chimeric) viral particles, such as hybrid viral capsids.

The AAV or Ad viral particles may also for instance have been mutated on one or more amino-acid residues, such as for instance one or more tyrosine residues.

In one embodiment, the adenovirus vector is selected from the group consisting of: wild-type Ad vectors, hybrid Ad vectors and mutant Ad vectors.

In another embodiment, the adeno-associated virus vector is selected from the group consisting of: wild-type AAV vectors, hybrid AAV vectors and mutant AAV vectors.

In further embodiments, the wild-type Ad vectors is Ad5 and the hybrid Ad vector is Ad5/F35.

In yet another embodiment, the hybrid AAV vector is AAV-DJ and the mutant AAV vector is AAV2-QuadYF.

In one embodiment, the vector or construct is synthetic mRNA, naked alphavirus RNA replicons or naked flavivirus RNA replicons.

In one embodiment, the lentiviral vector comprises a chimeric 5′ long terminal repeat (LTR) fused to a heterologous enhancer/promoter, such as the Rous Sarcoma Virus (RSV) or CMV promoter.

In one embodiment, the lentiviral vector comprises a deletion within the U3 region of the 3′ LTR, whereby said vector is replication incompetent and self-inactivating after integration.

In one embodiment, the one or more constructs or vectors are one or more plasmids.

In one embodiment, the backbone of the one or more constructs or vectors is selected from the group consisting of: FUW, pRRL-cPPT, pRLL, pCCL, pCLL, pHAGE2, pWPXL, pLKO, pHIV, pLL, pCDH and pLenti.

pRRL, pRLL, pCCL, and pCLL are lentivirus transfer vectors containing chimeric Rous sarcoma virus (RSV)-HIV or CMV-HIV 5′ LTRs, and vector backbones in which the simian virus 40 polyadenylation and (enhancerless) origin of replication sequences have been included downstream of the HIV 3′ LTR, replacing most of the human sequence remaining from the HIV integration site. In pRRL, the enhancer and promoter (nucleotides −233 to −1 relative to the transcriptional start site; GenBank accession no. J02342) from the U3 region of RSV are joined to the R region of the HIV-1 LTR. In pRLL, the RSV enhancer (nucleotides −233 to −50) sequences are joined to the promoter region (from position −78 relative to the transcriptional start site) of HIV-1. In pCCL, the enhancer and promoter (nucleotides −673 to −1 relative to the transcriptional start site; GenBank accession no. K03104) of CMV are joined to the R region of HIV-1. In pCLL, the CMV enhancer (nucleotides −673 to −220) is joined to the promoter region (position −78) of HIV-1.

The one or more constructs or vectors disclosed herein may comprise any type of element in addition to the polynucleotides encoding the TFs and the promoter region(s) driving expression of said TFs. For example, the one or more constructs or vectors may comprise regulatory, selectable and/or structural elements and/or sequences.

In one embodiment, the one or more constructs or vectors comprise self-cleaving peptides operably linked to at least two of the at least three coding regions, thus forming a single open reading frame. The self-cleaving peptide may be any type of self-cleaving peptide. In one embodiment, the self-cleaving peptide is a 2A peptide. In one embodiment, the 2A peptide is selected from the group consisting of equine rhinitis A virus (E2A), foot-and-mouth disease virus (F2A), porcine teschovirus-1 (P2A) and Thosea asigna virus (T2A) peptides.

In one embodiment, the one or more constructs or vectors comprises a posttranscriptional regulatory element (PRE) sequence. In a preferred embodiment, the PRE sequence is a Woodchuck hepatitis virus posttranscriptional regulatory element (WPRE).

In one embodiment, the one or more constructs or vectors comprise 5′ and 3′ terminal repeats. In a preferred embodiment, at least one of the 5′ and 3′ terminal repeats is a lentiviral long terminal repeat or a self-inactivating (SIN) design with partially deleted U3 of the 3′ long terminal repeat.

In one embodiment, the one or more constructs or vectors comprise a central polypurine tract (cPPT).

In one embodiment, the one or more constructs or vectors comprise a nucleocapsid protein packaging target site. In a preferred embodiment, the protein packaging target site comprises a HIV-1 psi sequence.

In one embodiment, the one or more constructs or vectors comprise a REV protein response element (RRE).

The composition disclosed herein may further comprise additional components, such as components that improve the efficiency of reprogramming cells according to the methods disclosed herein. The additional components may be macromolecules, such as for instance proteins, for example cytokines.

Cytokines are small proteins (peptides) important in cell signaling. Cytokines cannot cross the lipid bilayer of cells to enter the cytoplasm but act through surface receptors modulating intra-cellular signaling pathways. They have been shown to be involved in autocrine, paracrine, and endocrine signaling as immunomodulating agents. Cytokines include chemokines, interferons, interleukins, lymphokines, and tumour necrosis factors.

In one embodiment, the composition further comprises one or more pro-inflammatory cytokines. In one embodiment, the composition further comprises one or more hematopoietic cytokines. In one embodiment, the composition further comprises one or more cytokines selected from the group consisting of: IFNβ, IFNγ, TNFα, IFNα, IL-1β, IL-6, CD40I, Flt3I, GM-CSF, IFN-λ1, IFN-ω, IL-2, IL-4, IL-15, prostaglandin 2, SCF and oncostatin M (OM). In a preferred embodiment, the one or more cytokines are selected from the group consisting of: IFNβ, IFNγ and TNFα.

The additional components may also include for example small molecules. Small molecules are low molecular weight molecules that include lipids, monosaccharides, second messengers, other natural products and metabolites, as well as drugs and other xenobiotics, distinct from macromolecules such as proteins. Small molecules have high level of cell permeability, are cheap to produce, easy to synthesis and standardize.

In one embodiment, the composition further comprises one or more small molecules.

The small molecules may be for example small molecules that work as epigenetic modulators. The small molecules may be also for example small molecules targeting the epigenetic regulation of gene expression, such as for example histone deacetylase inhibitors (HDACi), DNA methyltransferase inhibitors, Histone methyltransferase (HMT) inhibitors or Histone demethylase inhibitor.

Thus, in one embodiment, the composition further comprises one or more histone deacetylase inhibitors.

In one embodiment, the composition further comprises valproic acid, suberoylanilide hydroxamic acid (SAHA), trichostatin A (TSA), sodium butyrate.

Thus, in one embodiment, the composition further comprises one or more DNA methyltransferase inhibitors, such as 5′-azacytidine (5′-azaC) or RG108.

In one embodiment, the composition further comprises one or more histone methyltransferase (HMT) inhibitors, such as BIX-01294, an inhibitor of inhibition of G9a-mediated H3K9me2 methylation.

In one embodiment, the composition further comprises one or more histone demethylase inhibitor, such as parnate (LSD1 inhibitor).

Such additional components may also include for example nucleic acids encoding additional TFs or genes associated with successful reprogramming.

Thus, in one embodiment, the composition further comprises one or more additional TFs and/or genes encoding one or more additional TFs, wherein the one or more TFs are associated with successful reprogramming. In one embodiment, the one or more TFs associated with successful reprogramming are selected from the TFs associated with successful reprogramming listed in Table 1.

In one embodiment, the composition further comprises one or more additional TFs and/or genes encoding additional TFs associated with successful reprogramming, wherein the one or more TFs associated with successful reprogramming are selected from the list in Table 1.

In one embodiment, the composition comprises a cell expressing one or more additional surface markers, wherein the one or more additional surface markers are selected from the surface markers listed in Table 1.

TABLE 1
List of genes associated with successful cDC1 reprogramming encoding Transcriptional regulators and surface markers.
Transcriptional regulators

| | | | | | | | | |
|---|---|---|---|---|---|---|---|---|
| PRDM2 | RREB1 | ARHGAP5 | OGT | ARID3B | TCERG1 | SIN3A | UBE2D3 | ZNF324 |
| SPEN | HIVEP1 | MIS18BP1 | RLIM | CIITA | NSD1 | RRN3 | EXOSC9 | TGIF2 |
| PNRC2 | KDM1B | ARID4A | BRWD3 | FBRS | DEK | ARHGAP17 | NAA15 | PABPC1L |
| GMEB1 | E2F3 | FOXN3 | ZMAT1 | SETD1A | PBX2 | NFATC2IP | PRMT9 | PRMT2 |
| ZNF362 | SOX4 | YY1 | ELF4 | IRF8 | BRD2 | ZNF689 | BDP1 | MED15 |
| AGO1 | ZKSCAN8 | RCOR1 | ZFY | ARRB2 | PHF1 | KAT8 | CCNH | CBX6 |
| SNIP1 | POU5F1 | COPS2 | KDM5D | PER1 | PPARD | ZDHHC1 | PPP2CA | ZFX |
| RLF | ZNF451 | RAB8B | SFPQ | JUNB | RUNX2 | EXOSC6 | TAF11 | HDAC8 |
| FOXJ3 | ZNF292 | USP3 | MYCL | LYL1 | PHIP | TERF2IP | CCNC | HTATSF1 |
| MYSM1 | ZBTB24 | ZNF592 | RBM15 | ELL | PNRC1 | BANP | HOXA9 | RBMX |
| HIPK1 | HSF2 | ALPK3 | CRTC2 | NFKBID | BCLAF1 | ZNF778 | ZNF655 | HCFC1 |
| TRIM33 | NCOA7 | CHD2 | AIM2 | ZFP36 | FOXK1 | MNT | POLR2J3 | MYRFL |
| CSDE1 | TNFAIP3 | MEF2A | BATF3 | DEDD2 | ZNF394 | ELP5 | DNAJC2 | CTNNBIP1 |
| TXNIP | ARID1B | CREBBP | NLRP3 | ZNF296 | KMT2E | KDM6B | ING3 | CDKN2C |
| GATAD2B | EZR | CNOT1 | ID2 | FOSB | ZNF800 | FLII | TOX | DR1 |
| ZBTB7B | PHF10 | NFAT5 | GRHL1 | HCK | PARP12 | ZNF207 | MTDH | DEDD |
| MEF2D | RBAK | WWP2 | KLF11 | STK4 | ZNF467 | CDK12 | MED30 | ZFP36L2 |
| ETV3 | ZNF316 | GSE1 | FOXN2 | NFATC2 | PRKAG2 | HEXIM1 | XPA | CTDSP1 |
| USF1 | AHR | HIC1 | BCL11A | APOBEC3A | EGR3 | TOB1 | ANP32B | PTMA |
| POGK | CDK13 | TOP3A | REL | ZNF711 | PDP1 | DDX5 | CDK9 | SS18L2 |
| POU2F1 | ZMIZ2 | PHF12 | PELI1 | BEX2 | TRPS1 | SAP30BP | SET | MAPKAPK3 |
| DHX9 | ZNF713 | MLLT6 | SERTAD2 | TSC22D3 | TRIB1 | USP36 | TAF3 | RBM15B |
| ELF3 | ZNF736 | MSL1 | PLEK | RUNX3 | CHRAC1 | MAFG | MLLT10 | PHF7 |
| ELK4 | RABGEF1 | STAT5B | NR4A2 | IKZF1 | AGO2 | TSHZ1 | CCAR1 | ABTB1 |
| RCOR3 | AUTS2 | STAT5A | CSRNP1 | NOD2 | PARP10 | ZNF236 | TIAL1 | CNBP |
| LBR | SMURF1 | ATXN7L3 | HCLS1 | CBFA2T3 | ZNF7 | SAFB | NAT10 | ING2 |
| TAF5L | CUX1 | KANSL1 | ZXDC | SPIB | UHRF2 | KHSRP | ZNF408 | MED10 |
| TARBP1 | KMT2C | ZNF652 | RNF168 | BTK | TOPORS | XAB2 | SART1 | SUB1 |
| ATAD2B | HMBOX1 | KAT7 | TET2 | UTY | KLF9 | ZNF121 | KMT5B | BTF3 |
| NCOA1 | KAT6A | MBTD1 | MEF2C | PADI4 | POLE3 | ILF3 | NFRKB | ANKRA2 |
| MXD1 | ZBTB10 | VEZF1 | TCF7 | PPP1R16B | ZBTB43 | RAB8A | PPHLN1 | HDAC3 |
| TET3 | YWHAZ | MED13 | KDM3B | ARID1A | ABL1 | USF2 | DAZAP2 | THAP1 |
| KCMF1 | UBR5 | ERN1 | PPARGC1B | GPBP1L1 | SFMBT2 | ERF | IKZF4 | TERF1 |
| KDM3A | RAD21 | HELZ | CNOT8 | GTF2B | CREM | CIC | CCDC59 | MAF1 |
| SAP130 | SMARCA2 | CBX4 | JARID2 | ZNF326 | ZNF487 | RELB | EID3 | OSTF1 |
| CCNT2 | RFX3 | RBBP8 | MED23 | ZNF644 | DDX21 | ZNF865 | DDX54 | CIZ1 |
| EPC2 | KDM4C | MBD2 | HDAC9 | MTF2 | ECD | RBM39 | TDRD3 | EGR2 |
| SP3 | RNF38 | MALT1 | SAP25 | DNTTIP2 | SPI1 | CTNNBL1 | ING1 | IRF7 |
| INO80D | ZBTB34 | ZNF407 | KDM7A | POGZ | CLP1 | SON | EAPP | ESRRA |
| BRPF1 | WAC | ZNF516 | EZH2 | ASH1L | MED17 | BRWD1 | BAZ1A | AIP |
| SATB1 | SIRT1 | ADNP2 | REPIN1 | YY1AP1 | FLI1 | PATZ1 | ZBTB25 | CLNS1A |
| KAT2B | KAT6B | DOT1L | CHD7 | ZNF281 | ZBTB44 | MCM5 | MED6 | CERS5 |
| TRAK1 | ZMIZ1 | ZBTB7A | KLF10 | MDM4 | YAF2 | MAFF | ELMSAN1 | PFDN5 |
| SETD2 | PTEN | PKN1 | TOP1MT | TRIM11 | ZNF641 | ATF4 | SNW1 | NAB2 |
| WDR82 | BTAF1 | MED26 | NR4A3 | ARID4B | KANSL2 | SREBF2 | PAPOLA | YEATS4 |
| ATXN7 | NFKB2 | GATAD2A | KLF4 | ZNF669 | SMARCD1 | HDAC10 | MTA1 | TDG |
| RYBP | EDRF1 | ZNF329 | AKNA | ZNF672 | NR4A1 | MSL3 | RTF1 | GTF3A |
| ZBTB11 | NUP98 | ZNF8 | NRARP | C1D | ZNF385A | BCOR | SLTM | HMGB1 |
| USF3 | ARNTL | ITCH | KLF6 | NMI | BAZ2A | ELK1 | ZFAND6 | MED4 |
| MSL2 | LMO2 | RBL1 | EPC1 | CIR1 | MBD6 | TSPYL2 | ZNF263 | HNRNPC |
| ATR | TRAF6 | SRC | JMJD1C | ANKAR | CNOT2 | RAP2C | LITAF | MAX |
| TSC22D2 | ZFP91 | RPRD1B | PCGF5 | SF3B1 | BTG1 | MECP2 | POLR3E | RAB11A |
| PHC3 | KDM2A | CHD6 | IKZF5 | TYW5 | NR2C1 | RAB25 | FUS | SMAD3 |
| BCL6 | EED | NCOA3 | ASCL2 | CREB1 | RNF10 | PAX4 | CBFB | ISL2 |
| RNF4 | KDM5A | NRIP1 | EHF | LRRFIP1 | MLXIP | ZBTB17 | NCOR1 | RNPS1 |
| TADA2B | ZNF384 | BACH1 | RPS6KA4 | NR1D2 | CCDC62 | TCEA3 | AATF | NAA60 |
| CENPC | ETV6 | PAXBP1 | LRRK2 | THRB | SFSWAP | KHDRBS1 | NOL11 | PYCARD |
| CNOT6L | CDKN1B | RUNX1 | VDR | CNOT10 | PSPC1 | THRAP3 | PRKAR1A | E2F4 |
| NFKB1 | AEBP2 | ETS2 | NACA | SMARCC1 | ZMYM5 | PLK3 | SS18 | NFATC3 |
| ELF2 | ARID2 | ZBTB21 | ZBTB39 | FOXP1 | FOXO1 | RFX5 | SAFB2 | HDAC5 |
| MAML3 | CCNT1 | APOBEC3G | NAP1L1 | NFKBIZ | ARGLU1 | TDRKH | ZGLP1 | JMJD6 |
| FBXW7 | KMT2D | EP300 | KDM2B | ZNF639 | PARP2 | ARID5A | CDKN2D | SRSF2 |
| MIER3 | USP15 | TCF20 | ZMYM2 | RBPJ | RALGAPA1 | AFF3 | CC2D1A | SIRT7 |
| PIK3R1 | CDK17 | ZBED4 | ELF1 | KLF3 | ZFP36L1 | ERCC3 | CEBPG | TGIF1 |
| NFKBIB | ZNF665 | XBP1 | HMGXB4 | SUV39H1 | BRCA2 | CHD1 | ZNF606 | USP12 |
| CHAF1A | SNRPB | EWSR1 | RBBP7 | PQBP1 | OGT | ANP32A | DBP | TAF1 |
| GPBP1 | FEM1B | MYNN | ZNF524 | SERTAD1 | NR3C1 | NCOR2 | PHF8 | FOS |
| ZNF131 | PIAS1 | TOP2B | NR1H2 | FBL | ANKHD1 | ZCCHC8 | KDM6A | NFKBIA |
| NIPBL | BRF1 | EAF1 | CRX | SSBP4 | JADE2 | SETD1B | TAB3 | ANKRD10 |
| HNRNPD | CCNK | RAF1 | SERTAD3 | FEM1A | AFF4 | MED13L | RPS6KA3 | RGCC |
| HMGN1 | NELFCD | ASCC2 | UXT | UIMC1 | TLE3 | TNIP2 | CREBRF | ASB2 |
| POLR2B | RPS6KA5 | CARF | | | | | | |

Surface Markers

| | | | | | | | | |
|---|---|---|---|---|---|---|---|---|
| LRRC8C | HLA-C | HLA-DQA1 | PHB2 | ITGA4 | SELL | ACAA1 | THBD | LRRC25 |
| TOR1AIP1 | HLA-B | PTGDR2 | A2M | CALCRL | BTLA | CD47 | IFNAR2 | FCGRT |
| PTPRC | HLA-DRA | JAML | PCBP2 | SLC9A9 | HLA-DRB5 | EPHB3 | ICOSLG | IL17RA |
| HNRNPU | HLA-DRB1 | MPZL2 | MPHOSPH9 | SLC2A9 | IL10RA | PLPP1 | ITGB2 | CD80 |
| BIRC6 | HLA-DQB1 | SIGLEC9 | LGMN | CD38 | ST14 | SLC38A9 | CSF2RA | SIGLEC1 |
| SLC8A1 | HLA-DPA1 | SIGLEC7 | DISP2 | AREG | LPAR5 | SSR1 | CD53 | CD58 |
| ST3GAL5 | HLA-DPB1 | HLA-DQA2 | HEXA | PARM1 | CLEC12A | BTN3A3 | FCRL6 | TOR3A |
| CD302 | ENPP1 | CLEC17A | SEMA7A | SLC39A8 | FLT3 | LEMD2 | CD48 | EPCAM |
| CERS6 | IFNGR1 | HLA-DQB2 | NAGPA | TLR3 | GPR65 | TNFRSF21 | SLAMF7 | GGCX |
| AGPS | TSPAN13 | MMRN1 | RSL1D1 | SLC12A7 | PLD4 | SUN1 | IL27RA | CLN3 |
| PGAP1 | CCDC126 | CD5 | CD68 | EMB | ITGAL | CHPF2 | ADGRE5 | EMILIN2 |
| SLC1A3 | CPVL | P2RX1 | TRPV2 | ERAP2 | CDH1 | ASAH1 | BST2 | CD226 |
| LNPEP | GUSB | CD22 | GRN | ST8SIA4 | PECAM1 | HGSNAT | CD37 | PTBP1 |
| SDK1 | FGL2 | CLCN6 | LAMA5 | CD74 | CD33 | HNRNPK | SIGLEC10 | TMEM259 |
| CPQ | PILRA | TACSTD2 | SUN2 | CD83 | LILRA5 | TSPAN15 | MILR1 | BRI3BP |
| GPR107 | TSPAN33 | ATP1A1 | NAGA | HLA-F | CSF2RB | LIPA | TTYH2 | TSHR |
| VSIR | ZC3HAV1 | TOR1AIP2 | CLEC1A | HLA-A | CXCR3 | NCAM1 | CYTH1 | SLC12A6 |
| ITPRIP | ENTPD1 | CD55 | ADAM15 | HLA-E | CD1C | LTBR | ICAM3 | NOMO3 |
| PTPRJ | TPP1 | ADAM17 | NCSTN | MAN2B1 | IL18R1 | ITGAX | ERMP1 | ATF6B |
| SORL1 | CADM1 | RNF149 | ATP1B1 | SLC23A2 | LY75 | ADCY7 | DPP7 | SRSF3 |
| SLC38A1 | CD4 | HS6ST1 | RPL32 | SULF2 | P2RY11 | SERPINF1 | PSAP | TMEM248 |
| GNPTAB | CD69 | SLC6A6 | SCAP | SLC9A7 | ICAM1 | CXCL16 | M6PR | CD36 |
| CHST11 | CLEC2B | NKTR | PLXND1 | MMGT1 | ZNF844 | EVI2A | P2RX7 | SYPL1 |
| SLC8B1 | PLBD1 | CLDND1 | ATP1B3 | TNFRSF14 | GAA | LTB4R | LMBRD1 | RPS23 |
| CEACAM1 | RPL18 | LAIR1 | APMAP | PIGT | XRCC6 | CD180 | ITGAM | CDH5 |
| UQCRC2 | ITFG1 | RPL19 | ABCA5 | INSR | P2RX4 | RPL21 | IDH2 | RPS15A |
| CD1D | PLBD2 | ITGB7 | PPM1L | RPL35A | PPT1 | PTGER3 | ABCA7 | INTS12 |
| SEMA4A | TSPAN2 | ADCY3 | ERO1B | CREG1 | TAGLN2 | SLCO3A1 | ATP6V0A2 | TLR1 |
| RPL9 | PDE4B | TNFRSF11A | CES1 | SCARB1 | LTB | CD7 | SERPINA1 | SLC12A9 |
| ADAM19 | | | | | | | | |

As stated above, the composition may further comprise genes encoding proteins associated with successful reprogramming. In one embodiment, said genes encode proteins other than TFs.

Expression of genes can be tested using methods known in the art, such as for example transcriptomics or other methods described herein.

In one embodiment, the composition is a pharmaceutical composition.

### Cell

Provided herein is a cell comprising one or more constructs or vectors, which upon expression encodes the transcription factors:
a) BATF3, or a biologically active variant thereof, wherein the biologically active variant is at least 70% identical to SEQ ID NO: 10 (BATF3);
b) IRF8, or a biologically active variant thereof, wherein the biologically active variant is at least 70% identical to SEQ ID NO: 11 (IRF8);
c) PU.1, or a biologically active variant thereof, wherein the biologically active variant is at least 70% identical to SEQ ID NO: 12 (PU.1);
d) IRF7, or a biologically active variant thereof, wherein the biologically active variant is at least 70% identical to SEQ ID NO: 21 (IRF7);
e) BATF, or a biologically active variant thereof, wherein the biologically active variant is at least 70% identical to SEQ ID NO: 19 (BATF);
f) SPIB, or a biologically active variant thereof, wherein the biologically active variant is at least 70% identical to SEQ ID NO: 23 (SPIB);
g) SPIC, or a biologically active variant thereof, wherein the biologically active variant is at least 70% identical to SEQ ID NO: 25 (SPIC); and/or
h) CEBPα, or a biologically active variant thereof, wherein the biologically active variant is at least 70% identical to SEQ ID NO: 13 (CEBPα); or any combination thereof;

wherein the one or more constructs or vectors comprise a promoter region capable of controlling the transcription of the transcription factors, wherein the promoter region comprises spleen focus-forming virus (SFFV) promoter, MND (myeloproliferative sarcoma virus enhancer, negative control region deleted, dl587rev primer-binding site substituted) promoter, CAG (CMV early enhancer/chicken β actin) promoter, cytomegalovirus (CMV) promoter, ubiquitin C (UbC) promoter, EF-1 alpha (EF-1α) promoter, EF-1 alpha short (EF1S) promoter, EF-1 alpha with intron (EF1i) promoter, phosphoglycerate kinase (PGK) promoter, or a promoter exhibiting essentially the same effect.

In one embodiment, the cell comprises:
a) one construct or vector which upon expression encodes the transcription factors BATF3, IRF8 and PU.1;
b) one construct or vector which upon expression encodes the transcription factors BATF3, IRF8 and SPIB;
c) a first construct or vector which upon expression encodes the transcription factors BATF3 and IRF8, and a second construct or vector which upon expression encodes the transcription factor PU1;
d) a first construct or vector which upon expression encodes the transcription factors BATF3 and IRF8, and a second construct or vector which upon expression encodes the transcription factor SPIB;
e) a first construct or vector which upon expression encodes the transcription factor BATF3, and a second construct or vector which upon expression encodes the transcription factors IRF8 and PU.1
f) a first construct or vector which upon expression encodes the transcription factor BATF3, and a second construct or vector which upon expression encodes the transcription factors IRF8 and SPIB;
g) a first construct or vector which upon expression encodes the transcription factor IRF8, and a second construct or vector which upon expression encodes the transcription factors BATF3 and PU.1;
h) a first construct or vector which upon expression encodes the transcription factor IRF8, and a second construct or vector which upon expression encodes the transcription factors BATF3 and SPIB;
i) a first construct or vector which upon expression encodes the transcription factor BATF3; a second construct or vector which upon expression encodes the transcription factor IRF8; and a third construct or vector which upon expression encodes the transcription factor PU.1; and/or
j) a first construct or vector which upon expression encodes the transcription factor BATF3; a second construct or vector which upon expression encodes the transcription factor IRF8; and a third construct or vector which upon expression encodes the transcription factor SPIB;

The TFs may be as defined herein in the section “Transcription factors”.

The promoter region may be as defined herein in the section “Promoters”.

The one or more constructs or vectors may be as defined herein in the section “Compositions”.

The cell may be any type of cell. In one embodiment, the cell is a mammalian cell. In one embodiment, the cell is a human cell. In another embodiment, the cell is a murine cell.

In one embodiment, the cell is selected from the group consisting of: a stem cell, a differentiated cell and a cancer cell.

In one embodiment, the stem cell is selected from the group consisting of: a pluripotent stem cell, an endoderm-derived cell, a mesoderm-derived cell, an ectoderm-derived cell and a multipotent stem cell, such as a mesenchymal stem cell and a hematopoietic stem cell.

In one embodiment, the differentiated cell is a cancer cell, such as for example a solid tumor cell, a hematopoietic tumor cell, a melanoma cell, a bladder cancer cell, a breast cancer cell, a lung cancer cell, a pleural cancer cell, a colon cancer cell, a rectal cancer cell, a colorectal cancer cell, a prostate cancer cell, a liver cancer cell, a pancreatic cancer cell, a bile duct cancer cell, a stomach cancer cell, a testicular cancer cell, a brain cancer cell, an ovarian cancer cell, a lymphatic cancer cell, a lymphoma cancer cell, a sarcoma cancer cell, a skin cancer cell, a brain cancer cell, a bone cancer cell, an oral cavity cancer cell, an head and neck cancer cell, or a soft tissue cancer cell, such as a glioblastoma cell, rectal carcinoma cell, or a mesothelioma cell.

In one embodiment, the differentiated cell is any somatic cell.

In one embodiment, the somatic cell is selected from the group consisting of: a fibroblast and a hematopoietic cell, such as a monocyte.

The cell disclosed herein may further be engineered or modified in a way that improves reprogramming efficiency according to the methods disclosed herein in the section “Methods”. Such modifications may for example include the overexpression or silencing of genes encoding TFs associated with successful reprogramming, respectively. It may also include the overexpression or silencing of other genes associated with reprogramming efficiency, such as overexpression or silencing of genes which are differentially expressed upon expression of the TFs disclosed herein in the section “Transcription factors”. Methods for overexpressing or silencing genes are well known in the art.

In one embodiment, the cell is engineered to overexpress one or more genes encoding TFs associated with successful reprogramming, such as for example one or more genes encoding TFs associated with successful reprogramming listed in Table 1. In one embodiment, the cell is engineered to overexpress one or more genes encoding TFs associated with successful reprogramming, wherein the one or more genes encoding TFs associated with successful reprogramming are selected from the list in Table 1.

### Methods

Provided herein is a method of reprogramming or inducing a cell into a dendritic or antigen-presenting cell, the method comprising the following steps:
a) transducing a cell with one or more constructs or vectors, which upon expression encodes the transcription factors:
   i) BATF3, or a biologically active variant thereof, wherein the biologically active variant is at least 70% identical to SEQ ID NO: 10 (BATF3);
   ii) IRF8, or a biologically active variant thereof, wherein the biologically active variant is at least 70% identical to SEQ ID NO: 11 (IRF8);
   iii) PU.1, or a biologically active variant thereof, wherein the biologically active variant is at least 70% identical to SEQ ID NO: 12 (PU.1);
   iv) IRF7, or a biologically active variant thereof, wherein the biologically active variant is at least 70% identical to SEQ ID NO: 21 (IRF7);
   v) BATF, or a biologically active variant thereof, wherein the biologically active variant is at least 70% identical to SEQ ID NO: 19 (BATF);
   vi) SPIB, or a biologically active variant thereof, wherein the biologically active variant is at least 70% identical to SEQ ID NO: 23 (SPIB);
   vii) SPIC, or a biologically active variant thereof, wherein the biologically active variant is at least 70% identical to SEQ ID NO: 25 (SPIC); and/or
   viii) CEBPα, or a biologically active variant thereof, wherein the biologically active variant is at least 70% identical to SEQ ID NO: 13 (CEBPα): wherein the one or more constructs or vectors comprise a promoter region capable of controlling the transcription of the transcription factors, wherein the promoter region comprises spleen focus-forming virus (SFFV) promoter, MND (myeloproliferative sarcoma virus enhancer, negative control region deleted, dl587rev primer-binding site substituted) promoter, CAG (CMV early enhancer/chicken β actin) promoter, cytomegalovirus (CMV) promoter, ubiquitin C (UbC) promoter, EF-1 alpha (EF-1α) promoter, EF-1 alpha short (EF1S) promoter, EF-1 alpha with intron (EF1i) promoter, phosphoglycerate kinase (PGK) promoter, or a promoter exhibiting essentially the same effect;
b) expressing the transcription factors;

whereby a reprogrammed or induced cell is obtained.

In one embodiment, the cell to be reprogrammed or induced is not a dendritic cell or an antigen-presenting cell.

In one embodiment, the reprogramming or induction is in vivo, such as in an animal or in a human.

In another embodiment, the reprogramming or induction is in vitro.

In another embodiment, the reprogramming or induction is ex vivo.

In one embodiment, the method further comprises a step of culturing the transduced cell in a cell media. The step of culturing the transduced cell in a cell media can be performed before or after step b) of the method, i.e. before or after expressing the transcription factors. In one embodiment, the step of culturing the transduced cells in a cell media is performed before expressing the transcription factors, i.e. after step a) and before step b) in the method presented herein. In one embodiment, the transduced cell is cultured during at least 2 days, such as at least 5 days, such as at least 8 days, such as at least 10 days, such as at least 12 days.

It may be beneficial for example for the efficiency of the reprogramming that the cell culture media contains one or more additional components.

In one embodiment, the method further comprises culturing the transduced cell in a media comprising one or more cytokines. In one embodiment, the one or more cytokines are pro-inflammatory cytokines. In one embodiment, the one or more cytokines are hematopoietic cytokines. In one embodiment, the one or more cytokines are selected from the group consisting of: IFNβ, IFNγ, TNFα, IFNα, IL-1β, IL-6, CD40I, Flt3I, GM-CSF, IFN-λ1, IFN-ω, IL-2, IL-4, IL-15, prostaglandin 2, SCF and oncostatin M (OM). In a preferred embodiment, the one or more cytokines are selected from the group consisting of: IFNβ, IFNγ and TNFα.

The method may further comprise culturing the transduced cell in a cell media comprising small molecules. The small molecules may be for example small molecules that work as epigenetic modulators. The small molecules may be also for example small molecules targeting the epigenetic regulation of gene expression, such as epigenetic modifiers, such as for example histone deacetylase inhibitors (HDACi), DNA methyltransferase inhibitors, Histone methyltransferase (HMT) inhibitors or Histone demethylase inhibitor, or any small molecule belonging to these categories such as the small molecules belonging to these categories disclosed herein.

In some embodiments, the method further comprises culturing the transduced cell in a a cell media comprising one or more histone deacetylase inhibitor(s).

In one embodiment, the one or more histone deacetylase inhibitor is (are) valproic acid.

In one embodiment, the cell is transduced with:
a) one construct or vector which upon expression encodes the transcription factors BATF3, IRF8 and PU.1;
b) one construct or vector which upon expression encodes the transcription factors BATF3, IRF8 and SPIB;
c) a first construct or vector which upon expression encodes the transcription factors BATF3 and IRF8, and a second construct or vector which upon expression encodes the transcription factor PU1;
d) a first construct or vector which upon expression encodes the transcription factors BATF3 and IRF8, and a second construct or vector which upon expression encodes the transcription factor SPIB;
e) a first construct or vector which upon expression encodes the transcription factor BATF3, and a second construct or vector which upon expression encodes the transcription factors IRF8 and PU1;
f) a first construct or vector which upon expression encodes the transcription factor BATF3, and a second construct or vector which upon expression encodes the transcription factors IRF8 and SPIB;
g) a first construct or vector which upon expression encodes the transcription factor IRF8, and a second construct or vector which upon expression encodes the transcription factors BATF3 and PU.1;
h) a first construct or vector which upon expression encodes the transcription factor IRF8, and a second construct or vector which upon expression encodes the transcription factors BATF3 and SPIB;
i) a first construct or vector which upon expression encodes the transcription factor BATF3; a second construct or vector which upon expression encodes the transcription factor IRF8; and a third construct or vector which upon expression encodes the transcription factor PU.1; and/or
j) a first construct or vector which upon expression encodes the transcription factor BATF3; a second construct or vector which upon expression encodes the transcription factor IRF8; and a third construct or vector which upon expression encodes the transcription factor SPIB;

The TFs may be as defined herein in the section “Transcription factors”.

The promoter region may be as defined herein in the section “Promoters”.

The one or more constructs or vectors may be as defined herein in the section “Compositions”.

The cell may be as defined herein in the section “Cell”.

The method may comprise additional steps that improve the reprogramming efficiency.

In one embodiment, the method further comprises overexpressing in the transduced cell one or more genes encoding TFs associated with successful reprogramming, such as for example one or more of the genes encoding the TFs associated with successful reprogramming listed in Table 1.

In one embodiment, the method further comprises overexpressing in the transduced cell one or more genes encoding TFs associated with successful reprogramming, wherein the one or more genes encoding TFs associated with successful reprogramming are selected from the list in Table 1.

In another embodiment, the method further comprises overexpressing in the transduced cell one or more genes encoding proteins associated with successful reprogramming. In one embodiment, said genes encode proteins other than TFs.

Overexpression and silencing of genes can be done using methods known in the art, such as for example by expressing the gene from a vector or by deleting part of the gene or the whole gene from the cell, respectively.

In one embodiment, the resulting reprogrammed cell is a type 1 conventional dendritic cell (DC1). cDC1s are a specialized subset of DCs, which for example express human leukocyte antigen-DR isotype (HLA-DR) and hematopoietic marker cluster differentiation 45 (CD45). cDC1s further have a typical RNA expression profile, and express surface markers cluster differentiation 141 (CD141), C-type lectin domain family 9 member A (CLEC9A), X-C Motif Chemokine Receptor 1 (XCR1) and cluster differentiation 226 (CD226).

Thus, in one embodiment, the resulting reprogrammed cell is enriched in one or more surface marker(s) selected from the list in Table 1.

In one embodiment, the resulting reprogrammed cell is CD45 positive. In one embodiment, the resulting reprogrammed cell is HLA-DR positive. In one embodiment, the resulting reprogrammed cell is CD141 positive. In one embodiment, the resulting reprogrammed cell is CLEC9A positive. In one embodiment, the resulting reprogrammed or induced cell is CD226 positive. In one embodiment, the resulting reprogrammed or induced cell is XCR1 positive. In one embodiment, the resulting reprogrammed or induced cell is CD45, HLA-DR, CD141, CLEC9A, XCR1 and/or CD226 positive.

Methods for determining whether or not a cell or cells are cDC1 cells are well known in the art. For example, one can determine whether said cells express CD45, HLA-DR, CD226, CD141, XCR1 and/or CLEC9A by incubating the cells with fluorophore-conjugated antibodies specific for CD45, HLA-DR, CD226, CD141, XCR1 and/or CLEC9A, and subsequentially screening the cells using flow cytometry. For example, one can determine whether said cells express one or more of the surface marker(s) selected from the list in Table 1 and identified as successful for reprogramming cells into cDC1, by incubating the cells with fluorophore-conjugated antibodies specific for said surface marker(s) and subsequentially screening the cells using flow cytometry. Furthermore, the RNA profile of the cells can be determined using single cell RNA seq, and used to classify the cell as a cDC1 if said RNA profile is identical or similar to that of a natural cDC1 cell. In addition, said cells can be characterized in terms of their functional properties, for example ability to response to TLR stimuli and up-regulate surface expression of CD40, CD80 and other co-stimulatory molecules, ability to secrete pro-inflammatory cytokines and chemokines and ability to activate antigen-specific T cells.

Thus, provided herein is a reprogrammed or induced cell obtained by the methods presented herein. In one embodiment, the cell is a dendritic or antigen-presenting cell.

### Transcription Factors

A transcription factor (TF) is a protein that controls the rate of transcription of genetic information from DNA to mRNA, by binding to a specific DNA sequence. The function of TFs is to regulate, i.e. turn on and off, the expression of genes. Groups of TFs function in a coordinated fashion to direct cell division, cell growth, and cell death throughout life; cell migration and organization during embryonic development; and intermittently in response to signals from outside the cell, such as a hormone. There are up to 1600 TFs in the human genome. Transcription factors are members of proteome as well as regulome.

TFs work alone or with other proteins in a complex, by promoting (as an activator), or blocking (as a repressor) the recruitment of RNA polymerase to specific genes. A defining feature of TFs is that they contain at least one DNA-binding domain (DBD), which attaches to a specific sequence of DNA adjacent to the genes that they regulate.

Presented herein are TFs that can be used to reprogram cells into a dendritic or antigen-presenting cell. Such TFs include BATF3, IRF8, PU.1, IRF7, BATF, SPIB, SPIC and CEBPA.

BATF3 is a nuclear basic leucine zipper that belongs to the AP-1/ATF superfamily of TFs. It controls the differentiation of CD8⁺ thymic conventional dendritic cells in the immune system. It acts via the formation of a heterodimer with the JUN family proteins that recognizes and binds a specific DNA sequence to regulate the expression of target genes.

IRF8 is a TF belonging to the interferon regulatory factor (IRF) family. It plays a role in the regulation of lineage commitment, and in myeloid cell maturation. IRF8, as well as other TFs in the IRF family, binds to the IFN-stimulated response element and regulates expression of genes stimulated by type I IFNs.

PU.1 is a TF belonging to the Erythroblast Transformation Specific (ETS)-domain family. It is a transcriptional activator that binds the PU-box, a purine-rich DNA sequence that can act as a lymphoid-specific enhancer. PU.1 may be specifically involved in the differentiation or activation of myeloid cells, such as macrophages and dendritic cells, as well as B-cells.

IRF7 is a TF belonging to the interferon regulatory factor (IRF) family. IRF7 has been shown to play a role in the transcriptional activation of virus-inducible cellular genes, including the type I interferon genes. IRF7 is constitutively expressed in lymphoid tissues and is inducible in many other tissues of the whole body.

BATF is a nuclear basic leucine zipper that belongs to the AP-1/ATF superfamily of TFs. BATF can interact with partner transcription factors, including IRF8 and IRF4, via the leucine zipper domain to mediate cooperative gene activation. Compensation among BATF factors has been previously demonstrated in the context of cDC1 development.

SPIB is a TF belonging to the Erythroblast Transformation Specific (ETS)-domain family. Like PU1, SPIB is a sequence-specific transcriptional activator that binds to the PU-box, a purine-rich DNA sequence that can act as a lymphoid-specific enhancer. Promotes development of plasmacytoid dendritic cells (pDCs) and cDC precursors.

SPIC is a TF belonging to the Erythroblast Transformation Specific (ETS)-domain family. Like PU.1 and SPIB, SPIC is a sequence-specific transcriptional activator that binds to the PU-box, a purine-rich DNA sequence. SPIC controls the development of red pulp macrophages required for red blood cell recycling and iron homeostasis.

CEBPα (CCAAT Enhancer Binding Protein Alpha) is a TF that contains a basic leucine zipper (bZIP) domain and recognizes the CCAAT motif in the promoters of target genes. CEBPα coordinates proliferation arrest and the differentiation of myeloid progenitors, adipocytes, hepatocytes, and cells of the lung and the placenta.

Also disclosed herein are biologically active variants of BATF3, IRF8, PU1, IRF7, BATF, SPIC and SPIB. A biologically active variant is a variant of said TF that retains at least some of the activity of the parent TF. For example, a biologically active variant of SPIB, SPIC, BATF, BATF3, IRF8, or PU.1 is able to induce and/or inhibit expression of the same genes as the parent BATF3, IRF8, or PU.1, respectively. Three biologically active variants of SPIB, SPIC, BATF, BATF3, IRF8, and PU.1 are able to reprogram or induce a cell into a dendritic or antigen-presenting cell according to the methods disclosed herein. However, a biologically active variant of the respective TF may be more or less efficient compared to the respective parent TF. For example, the efficiency of inducing and/or inhibiting expression of genes, and/or the efficiency of reprogramming or inducing a cell into a dendritic cell, may be increased or decreased compared to the respective parent TF.

In one embodiment, the biologically active variant of BATF3 is at least 60% identical to SEQ ID NO: 10, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 10.

In one embodiment, the biologically active variant of IRF8 is at least 60% identical to SEQ ID NO: 11, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 11.

In one embodiment, the biologically active variant of PU.1 is at least 60% identical to SEQ ID NO: 12, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 12.

In one embodiment, the biologically active variant of IRF7 is at least 60% identical to SEQ ID NO: 21 (IRF7), such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 21.

In one embodiment, the biologically active variant of BATF is at least 60% identical to SEQ ID NO: 19 (BATF), such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 19.

In one embodiment, the biologically active variant of SPIB is at least 60% identical to SEQ ID NO: 23 (SPIB), such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 23.

In one embodiment, the biologically active variant of SPIC is at least 60% identical to SEQ ID NO: 25 (SPIC), such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 25.

In one embodiment, the biologically active variant of CEBPA is at least 60% identical to SEQ ID NO: 13 (CEBPα), such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 13.

In one embodiment, BATF3 is encoded by a polynucleotide sequence with at least 60% sequence identity to SEQ ID NO: 14, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% such as 100% sequence identity to SEQ ID NO: 14.

In one embodiment, IRF8 is encoded by a polynucleotide sequence with at least 60% sequence identity to SEQ ID NO: 15, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 15.

In one embodiment, PU.1 is encoded by a polynucleotide sequence with at least 60% sequence identity to SEQ ID NO: 16, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 16.

In one embodiment, IRF7 is encoded by a polynucleotide sequence with at least 60% sequence identity to SEQ ID NO: 20, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% such as 100% sequence identity to SEQ ID NO: 20.

In one embodiment, BATF is encoded by a polynucleotide sequence with at least 60% sequence identity to SEQ ID NO: 18, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% such as 100% sequence identity to SEQ ID NO: 18.

In one embodiment, SPIB is encoded by a polynucleotide sequence with at least 60% sequence identity to SEQ ID NO: 22, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% such as 100% sequence identity to SEQ ID NO: 22.

In one embodiment, SPIC is encoded by a polynucleotide sequence with at least 60% sequence identity to SEQ ID NO: 24, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% such as 100% sequence identity to SEQ ID NO: 24.

In one embodiment, CEBPα is encoded by a polynucleotide sequence with at least 60% sequence identity to SEQ ID NO: 17, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 17.

### Promoters

A promoter or promoter region is a sequence of DNA to which proteins bind to initiate transcription of a single RNA from the DNA downstream of it. This RNA may be an mRNA which encodes a protein, or it may have a function itself, such as transfer-RNA (tRNA), or ribosomal RNA (rRNA). Promoters are located near the transcription start sites of genes, upstream of said gene on the DNA.

Eukaryotic promoter regions may, beside the core promoter, further comprise other elements such as for example a transcription start site (TSS); a binding site for RNA polymerase; TF binding sites; and other regulatory and/or structural elements. Eukaryotic gene promoter regions are typically located upstream of the gene and can have regulatory elements several kilobases away from the TSS. Such regulatory elements may for example be enhancers.

The TFs disclosed herein are controlled by promoter regions comprising core promoters. The inventors have surprisingly shown that reprogramming of cells according to the methods disclosed herein can be significantly improved by expressing the TFs disclosed herein under certain promoters or promoter regions. Such promoter regions include those comprising the SFFV promoter, the MND promoter, the CAG promoter, the CMV promoter, the EF-1α promoter, the EF1S promoter, the EF1i promoter, the PGK promoter, as well as other promoters exhibiting essentially the same effect.

A promoter or promoter region exhibiting essentially the same effect is defined herein as a promoter or promoter region that exhibits the same expression level of the gene(s) it controls as the promoters or promoter regions disclosed herein. Thus, whether or not a promoter region exhibits essentially the same effect as a promoter region disclosed herein can be measured by measuring the expression level of the gene(s) controlled by said promoter region and comparing it to the expression level of the same gene(s) controlled by the promoter region disclosed herein, wherein the expression level of the tested promoter region and the expression level of the promoter region disclosed herein are tested under the same conditions. Methods for measuring expression levels are well known in the art, and can be done using routine experimentation. For example, the expression level of a gene controlled by a certain promoter region can be measured by measuring the amount of messenger RNA (mRNA) generated by the expression of said gene. The amount of mRNA can for example be measured using reverse transcription-polymerase chain reaction (RT-PCR) or transcriptomics. The expression level of a gene controlled by a certain promoter region can also be measured by measuring the amount of protein, i.e. the amount of gene product, generated by expression of said gene using proteomics or Western blot. Herein, a promoter exhibiting essentially the same effect as the disclosed promoters regions are defined as promoter regions generating an expression level that is 50% higher or 50% lower than the expression levels of the promoter regions disclosed herein, such as 45%, such as 40%, such as 35%, such as 30%, such as 25%, such as 20%, such as 15%, such as 10%, such as 5% higher or lower than the expression levels of the promoters disclosed herein.

The TFs disclosed herein may be controlled by any of the disclosed promoter regions. In one embodiment, the same promoter region controls the expression of at least one TF, such as at least two TFs, such as three TFs. In one embodiment, a first promoter region controls the expression of a first TF; a second promoter region controls the expression of a second TF; and a third promoter region controls the expression of a third TF. In one embodiment, a first promoter region controls the expression of a first and a second TF, and a second promoter region controls the expression of a third TF. The TFs may be as disclosed herein in the section “Transcription factors”.

In one embodiment, the SFFV promoter comprises or consists of a polynucleotide sequence at least 70% identical to SEQ ID NO: 1, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 1.

In one embodiment, the MND promoter comprises or consists of a polynucleotide sequence at least 70% identical to SEQ ID NO: 2, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 2.

In one embodiment, the CAG promoter comprises or consists of a polynucleotide sequence at least 70% identical to SEQ ID NO: 3, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 3.

In one embodiment, the CMV promoter comprises or consists of a polynucleotide sequence at least 70% identical to SEQ ID NO: 4, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 4.

In one embodiment, the UbC promoter comprises or consists of a polynucleotide sequence at least 70% identical to SEQ ID NO: 5, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 5.

In one embodiment, the EF-1α promoter comprises or consists of a polynucleotide sequence at least 70% identical to SEQ ID NO: 6, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 6.

In one embodiment, the EF1S promoter comprises or consists of a polynucleotide sequence at least 70% identical to SEQ ID NO: 7, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 7.

In one embodiment, the EF1i promoter comprises or consists of a polynucleotide sequence at least 70% identical to SEQ ID NO: 8, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 8.

In one embodiment, the PGK promoter comprises or consists of a polynucleotide sequence at least 70% identical to SEQ ID NO: 9, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 9.

### Methods of Treatment

The compositions and methods disclosed herein may be used for treating and/or preventing diseases or disorders, such as for example tumours and cancers or infectious diseases.

In one embodiment, the compositions and methods disclosed herein are used in the treatment of a tumour and/or cancer or of infectious diseases.

In one embodiment, the tumour and/or cancer is selected from the group consisting of: a benign tumor, a malignant tumor, early cancer, basal cell carcinoma, cervical dysplasia, sarcoma, germ cell tumor, retinoblastoma, glioblastoma, lymphoma, Hodgkin's lymphoma, non-Hodgkin's lymphoma, blood cancer, prostate cancer, ovarian cancer, cervix cancer, oesophageal cancer, uterus cancer, vaginal cancer, breast cancer, head and neck cancer, gastric cancer, oral cavity cancer, naso-pharynx cancer, trachea cancer, larynx cancer, bronchi cancer, bronchioles cancer, lung cancer, pleural cancer, bladder and urothelial cancer, hollow organs cancer, esophagus cancer, stomach cancer, bile duct cancer, intestine cancer, colon cancer, colorectum cancer, rectum cancer, bladder cancer, ureter cancer, kidney cancer, liver cancer, gall bladder cancer, spleen cancer, brain cancer, lymphatic system cancer, bone cancer, pancreatic cancer, leukemia, chronic myeloid leukemia, acute lymphoblastic leukemia, acute myeloid leukemia, skin cancer, melanoma or myeloma. In a preferred embodiment, the cancer is selected from the group consisting of melanoma, head and neck cancer, bladder and urothelial cancer, pancreatic cancer and glioblastoma.

Accordingly, the cells produced by the methods described herein can be used to prepare cells to treat or alleviate several cancers and tumours including, but not limited to, breast cancer, prostate cancer, lymphoma, skin cancer, pancreatic cancer, colon cancer, melanoma, malignant melanoma, oesophageal cancer, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like.

Provided herein is the composition; the cell; the pharmaceutical composition; and/or the reprogrammed or induced cell as presented herein for use in medicine.

Also provided herein is the composition; the cell; the pharmaceutical composition; and/or the reprogrammed or induced cell as presented herein for use in the treatment of cancer or infectious diseases.

Further provided herein is a method of treating cancer or infectious diseases, the method comprising administering to an individual in need thereof the composition; the cell; the pharmaceutical composition; and/or the reprogrammed or induced cell as presented herein.

Also provided herein is the use of the composition; the cell; the pharmaceutical composition; and/or the reprogrammed or induced cell as presented herein, for the manufacture of a medicament for the treatment of cancer or infectious diseases.

### EXAMPLES

### Example 1. General Methods and Materials

### Cell Culture

Human embryonic kidney HEK293T cells, human embryonic fibroblasts (HEFs) (passage 3-8) and human dermal fibroblasts (HDFs) (passage 3-8) were maintained in growth medium Dulbecco's modified eagle medium (DMEM) supplemented with 10% (v/v) heat-inactivated fetal bovine serum (FBS), 2 mM L-Glutamine and antibiotics (10 U/ml Penicillin 10 μg/ml Streptomycin)—DMEM complete. Mouse embryonic fibroblasts (MEFs) (passage 3-5) were isolated from E13.5 embryos of Clec9a-tdTomato reporter mice as previously described (Rosa et al. 2020) and cultured in DMEM complete in 0.1% gelatin-coated dishes. Monocyte-derived dendritic cells (moDCs) were cultured in RPMI 1640 supplemented with 10% heat-inactivated FBS, 2 mM L-Glutamine and antibiotics—RPMI complete. cDC1s isolated from peripheral blood were maintained in RPMI complete supplemented with 50 μM 2-Mercaptoethanol, 1 mM sodium pyruvate and antibiotics. Mesenchymal Stromal Cells (MSCs, passage 3-5) were cultured in minimum essential media (MEM) supplemented with 10% of Pooled Human Platelet Lysate, 2 U/ml heparin (STEMCELL Technologies), 2 mM L-Glutamine and antibiotics. All tissue culture reagents were from Thermo Fisher Scientific unless stated otherwise.

### Mice

C57BL/6J and OT-I mice were acquired from Janvier and Taconic, respectively. Clec9a^{Cre/Cre}Rosa^{tdTomato/tdTomato} (Clec9a-tdTomato) animals were a kind gift of Caetano Reis e Sousa, Francis Crick Institute, London, United Kingdom (Rosa et al., 2018), and were re-derived by Janvier before import to Lund University animal house. All animals were housed under controlled temperature (23±2° C.), subject to a fixed 12-h light/dark cycle, with free access to food and water. Animal care and experimental procedures were performed in accordance with Swedish guidelines and regulations after approval from local committees.

### Lentiviral Production

HEK293T cells were co-transfected with a mixture of transfer plasmid, packaging and VSV-G-encoding envelope constructs with Polyethylenimine (PEI) as previously described (Rosa et al., 2020). Viral supernatants were harvested after 36, 48 and 72 hours, filtered (0.45 μm, low protein binding), concentrated 40-fold with Lenti-X Concentrator and stored at −80° C.

### Viral Transduction and Reprogramming

HEFs, HDFs and Clec9a-tdTomato MEFs were seeded at a density of 40,000 cells per well and MSCs at a density of 50,000 cells per well on 0.1% gelatin coated 6-well plates. On the following day, cells were incubated overnight with either a ratio of 1:1 TetO-PIB and M2rtTA, SFFV-PIB-GFP or SFFV-GFP lentiviral particles in media supplemented with 8 μg/ml polybrene. Cells were transduced overnight twice in consecutive days and media replaced in between. After the second transduction, media was replaced by normal growth media (day 0). When using TetO-PIB, media was supplemented with Dox (1 μg/ml). Media was changed every 2-3 days for the duration of cultures. When stated, media was supplemented with LPS (100 ng/ml, Enzo), Poly I:C (25 μg/ml, InvivoGen) and R848 (3 μg/ml, InvivoGen) overnight. Cytokines were added at day 2 and kept for the duration of cultures; For reprogramming in xeno-free conditions, MSCs were cultured in X-Vivo 15 (Lonza) after transduction and media changed every 2-3 days for the duration of cultures.

### Molecular Cloning

For the doxycycline (Dox)-inducible overexpression system, coding regions of human PU.1, IRF8 and BATF3 (PIB) were cloned in this order in the pFUW-TetO plasmid separated by 2A self-cleaving peptides. The first two coding sequences lacked the stop codon. Coding regions of PU.1, IRF8, BATF3, ID2, TXNIP, ZFP36PLEK, SUB1, JUNB, CREM, KLF4, MXD1, LITAF, IRF7, FOS, NMI, TFEC, SP110, IRF5, STAT2, BATF, ZNF267, IRF1, RELB and BATF2 were individually cloned in the pFUW-TetO plasmid. A lentiviral vector containing the reverse tetracycline transactivator M2rtTA under the control of constitutively active human ubiquitin C promoter (pFUW-UbC-M2rtTA) was used for co-transduction (Rosa et al. 2018). For constitutive overexpression, the human PIB polycistronic cassette was sub-cloned into lentiviral vectors with constitutive promoters: pFUW-UbC, pRRL.PPT-SFFV, pRRL.PPT-PGK, pRRL.PPT-EF1S, pHAGE2-EF1 and pWPXL-EF1i (Addgene plasmid #12257) (Sommer et al. 2009; Dahl et al. 2015; Schambach et al. 2006).

Generation of Human MSC, moDC and cDC1 Cultures

Human bone marrow (BM) cells were collected at the Hematology Department, Lund University (Sweden) from consenting healthy donors by aspiration from the iliac crest. The use of human samples was approved by the Institutional Review Board of Lund University in accordance with the Declaration of Helsinki. Mononuclear cells from BM aspiration samples were isolated by density gradient centrifugation (LSM 1077 Lymphocyte, PAA) with prior incubation with RosetteSep Human Mesenchymal Stem Cell Enrichment Cocktail (STEMCELL Technologies) for lineage depletion by magnetic activated cell sorting (MACS) (CD3, CD14, CD19, CD38, CD66b, glycophorin A) as previously described (Li et al. 2014). MSCs purification was followed by FACS sorting (additional information below). To generate moDCs, fresh leukocyte concentrates were diluted in phosphate-buffered saline (PBS) 1× at 1:1 ratio and peripheral blood mononuclear cells (PBMCs) were separated by density gradient centrifugation using Lymphoprep (STEMCELL Technologies). CD14⁺ monocytes were enriched from PBMCs by positive selection using MACS with CD14 microbeads (Miltenyi Biotec) according to manufacturer's protocol. CD14⁺ monocytes were cultured in X-VIVO 15 media (Lonza) supplemented with 5% FBS for 7 days. Cells were seeded at a density of 1×10⁶ cells/ml and 8 ml of cell suspension were added to a T75 flask. Culture media was supplemented with IL-4 (350 ng/ml) and GM-CSF (850 ng/ml) at day 0 and media replaced every 2-3 days. At day 6, IL-6 (15 ng/ml), PGE2 (10 μg/ml), TNF-α (10 ng/ml), and IL1β (5 ng/ml) were added to the culture media for 24 hours to generate mature moDCs. Mature moDCs were dissociated using TrypLE Express (Gibco) and used for functional characterization. To isolate cDC1s for functional assays, DCs were enriched from PBMCs by MACS using the Pan-DC enrichment kit (Miltenyi Biotec) followed by further purification with anti-CLEC9A antibody coupled with biotin and anti-biotin microbeads (Miltenyi Biotec).

### Flow Cytometry Analysis

For the analysis of surface marker expression, human and mouse cells were dissociated and incubated with antibodies diluted in PBS 5% FBS at 4° C. for 30 minutes in the presence of mouse or rat serum (1/100, GeneTex), for human and mouse cells respectively, to block unspecific binding. Cells were washed and resuspended in PBS 5% FBS and analyzed in BD FACSCanto II or BD LSRFortessa flow cytometers (BD Biosciences), unless stated otherwise. DAPI was used for dead cell exclusion. Flow cytometry data were analyzed using FlowJo software (FLOWJO, LLC, version 10.6.1). All flow cytometry analyses were performed in live single cell gates.

### Fluorescence Activated Cell Sorting (FACS)

DCs were enriched from PBMCs by negative selection using MACS using the Pan-DC Enrichment Kit (Miltenyi Biotec) according to manufacturer's protocol. HLA-DR⁺CD11C⁺CD141⁺ cDC1s, HLA-DR⁺CD11C⁺CD141⁻CD1C⁺ cDC2s and HLA-DR⁺CD11C⁻CD123⁺ pDCs were purified in a FACSAria III (BD Biosciences) and used for single-cell RNA-seq profiling. To purify CD45⁺, CD45⁺ HLA-DR⁻, CD45⁺ HLA-DR⁺ and CD45⁺HLA-DR⁺CD226⁺ hiDCs, cells were dissociated using TrypLE Express, resuspended in PBS 5% FBS, incubated at 4° C. for 30 minutes with anti-CD45, anti-HLA-DR and anti-CD226 antibodies in the presence of mouse serum and purified in a FACSAria III. For isolation of human primary MSCs, lineage-depleted BM mononuclear cells were incubated in blocking buffer [PBS without Ca2/Mg2, 3.3 mg/ml human normal immunoglobulin (Octapharma), 1% FBS], followed by antibody staining. CD45-CD271⁺ MSCs were purified in FACSAria Ill (BD Biosciences) and used for reprogramming experiments. Dead cells were excluded by 7-Amino-actinomycin staining (7-AAD) or 4′,6-diamidino-2-phenylindole (DAPI).

### Single-Cell RNA Sequencing

HEFs, hiDCs at day 3, 6 and 9 (CD45⁺HLA-DR⁻ and CD45⁺HLA-DR⁺), cDC1s, cDC2s and pDCs from peripheral blood (from 3 individual donors) were FACS sorted for scRNA-seq. Purified cells were loaded on a 10× Chromium (10× Genomics) according to manufacturer's protocol. scRNA-seq indexed libraries were prepared using Chromium Single Cell 3′ v2 and v3 Reagent Kit (10× Genomics) according to manufacturer's protocol. hiDCs at day 9 reprogrammed in the presence and absence of cytokines from HEFs and HDFs and CD45⁺HLA-DR⁺CD226⁺ hiDCs were also profiled. Library quantification and quality assessment was determined using Agilent Bioanalyzer using the High Sensitivity DNA analysis kit (Agilent). Indexed libraries were pooled at equimolarity and sequenced on an Illumina NextSeq 500. Coverage of approximately 130,000 reads per single cell was obtained. Details regarding scRNA-seq data analysis can be found in supplementary materials.

### Analysis of RNA Sequencing Data

In total, the transcriptome of 51,903 single-cells was profiled with approximately 130,000 reads per cell (R1 read: technical, length: 26 to 28 bp; R2 read: biological, length: 90 to 98 bp). Paired-end sequencing reads of single cell RNA-seq were processed using the 10× Genomics software Cell Ranger v2.2.0 (https://support.10×genomics.com/single-cell-gene-expression/software). Firstly, cellranger mkfastq was used to convert binary base call files to FASTQ files and to decode the multiplexed samples simultaneously. Next, cellranger count was applied to FASTQ files and alignment to human (hg38) genome assemblies using STAR v2.5.3a was performed. Then, the output files from each run were combined to produce one single matrix using cellranger aggr. The sparse expression matrix generated by cellranger analysis pipeline was used as input to Scater library (http://bioconductor.org/packages/release/bioc/html/scater), and cells and genes that passed quality control thresholds were included according to the following criteria: 1) total number of unique molecular identifiers (UMIs) detected per sample greater than 3 lower median absolute deviations (MADs); 2) number of genes detected in each single cell greater than 3 lower MADs; 3) percentage of counts in mitochondrial genes <7.5%. The resulting expression matrix was filtered by Scater analysis pipeline and used as input to the Seurat library v4 (https://satijalab.org/seurat). To account for technical variation, batch integration was performed. Firstly, each batch was normalized separately using “LogNormalize” with the scale factor of 10,000 and 9,000 variable features were identified. Next, batch integration was performed by finding corresponding anchors between the batches using 30 dimensions. Then, 50 principal components were computed and their significances tested by JackStraw. The first 30 principal components were selected for subsequent tSNE visualization. For differential expression analysis between cell types, Seurat v4 FindAllMarkers function was used with LR test with specification of latent variables (sequencing run and donor) to reduce the batch effects and defining following parameters: logfc.threshold=0.5, min.pct=0.5, BH-adjusted p<0.05. Additionally, scaling was performed with specification of latent variables in vars.to.regress parameter and the resulting genes were visualized using Seurat v4 DoHeatmap function. For additional samples, all data were normalized together using “LogNormalize” with the scale factor of 10,000. The first 30 principal components were selected for subsequent tSNE and UMAP visualization. For differential expression analysis comparing affiliated hiDC to controls, Seurat v4 FindMarkers function was used with Wilcox test and defining following parameters: logfc.threshold=0.25, min.pct=0.25, BH-adjusted p<0.05. The resulting intersections were visualized using Vennerable R library (https://github.com/js229/Vennerable).

### Example 2. PU.1, IRF8 and BATF3 Induce Global cDC1 Gene Expression Program in Human Fibroblasts

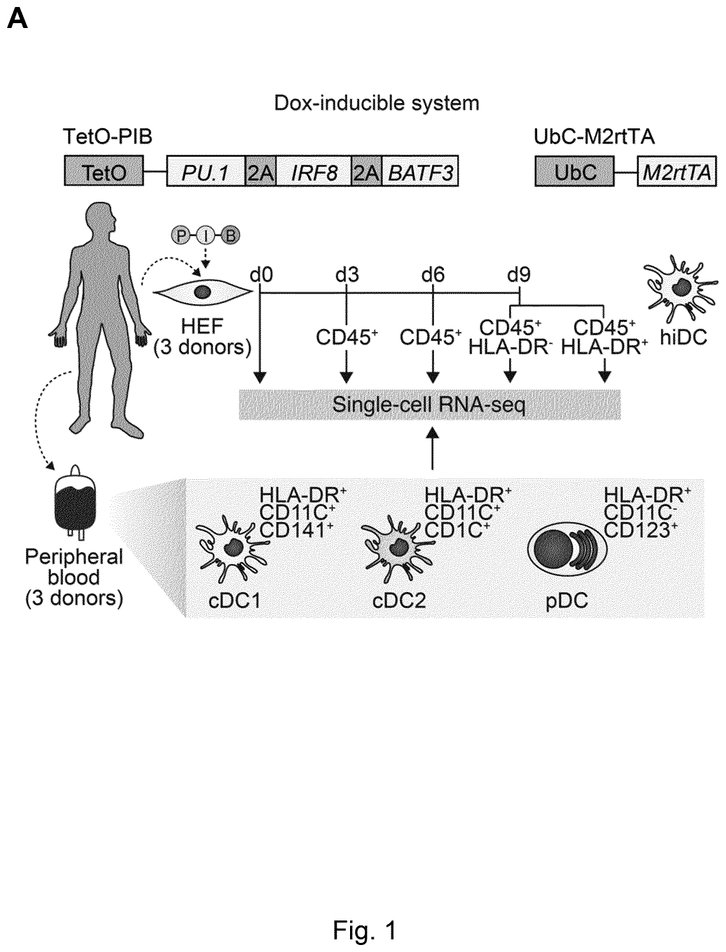

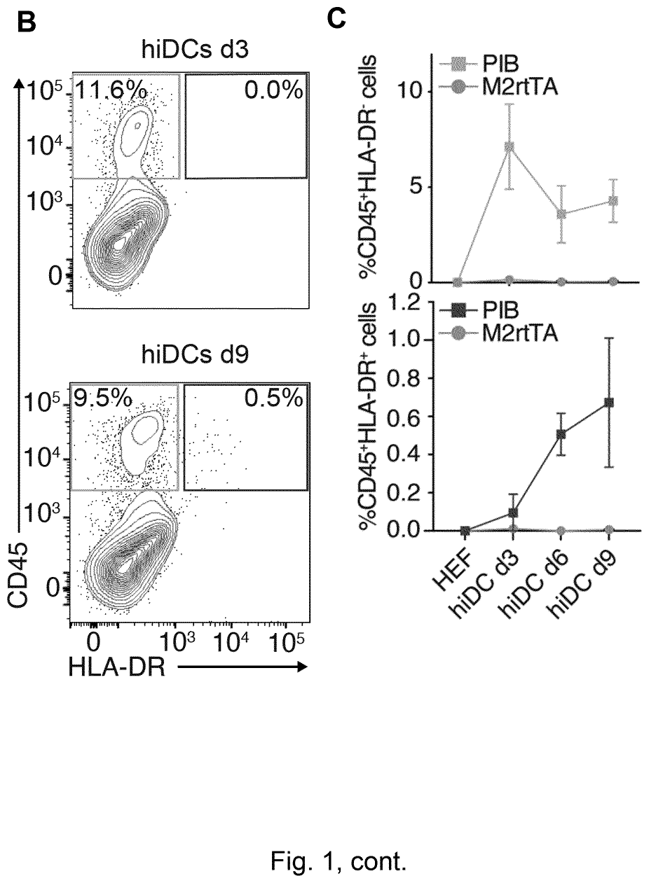

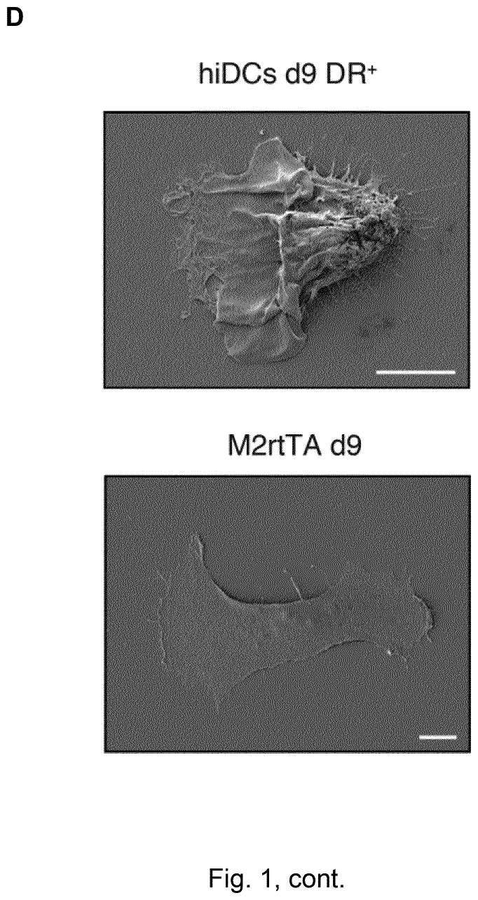

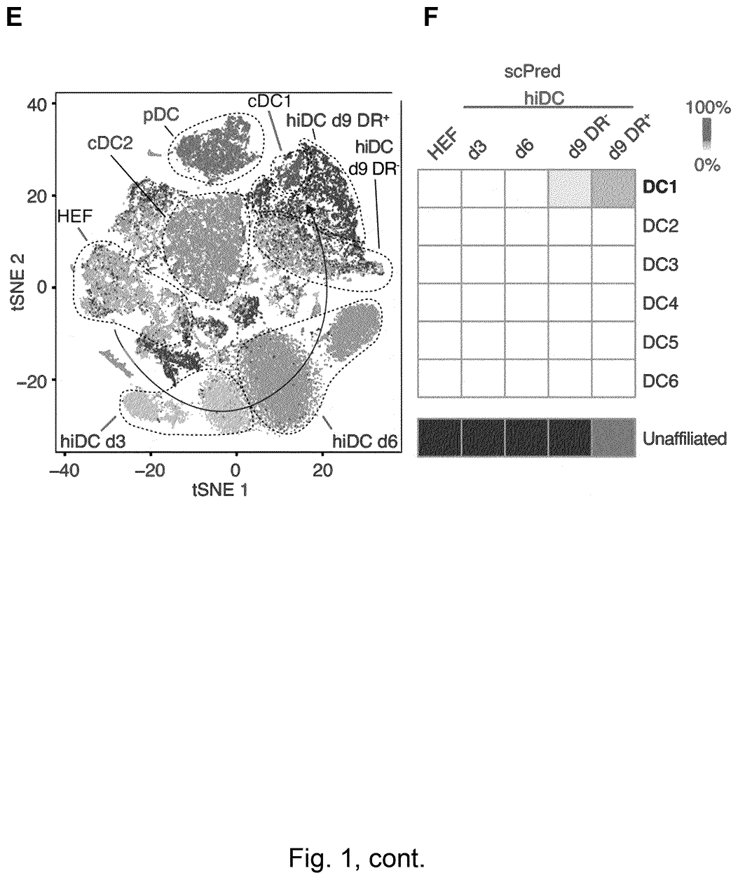

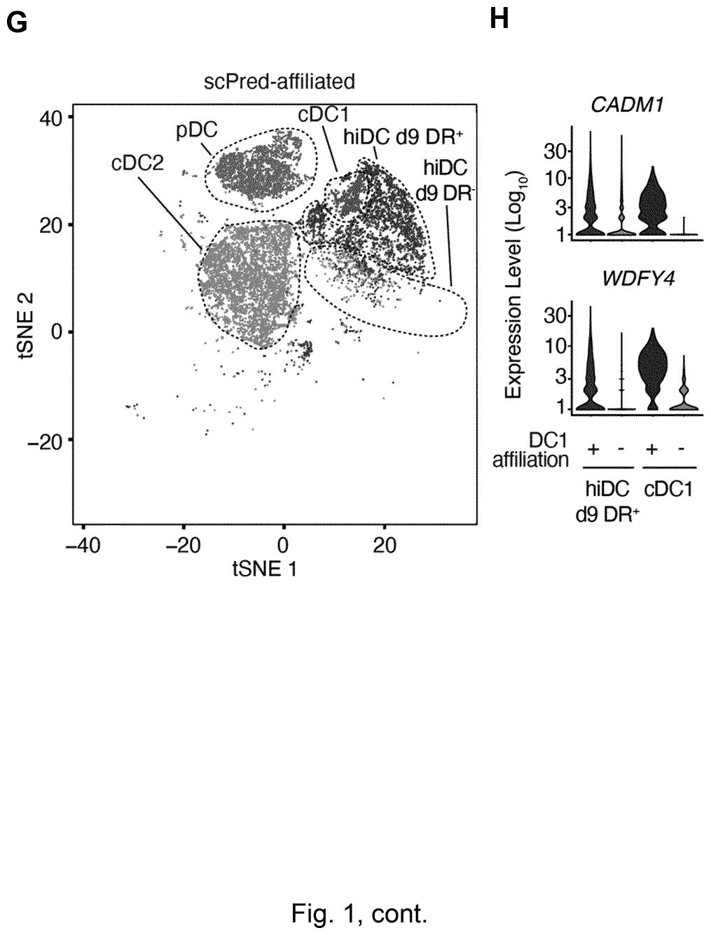

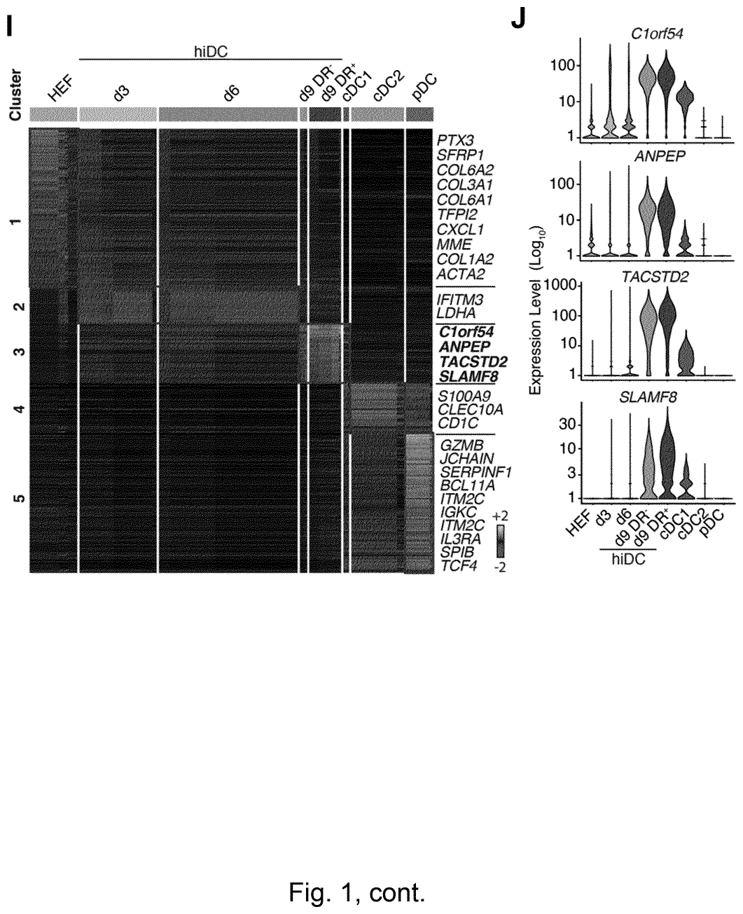

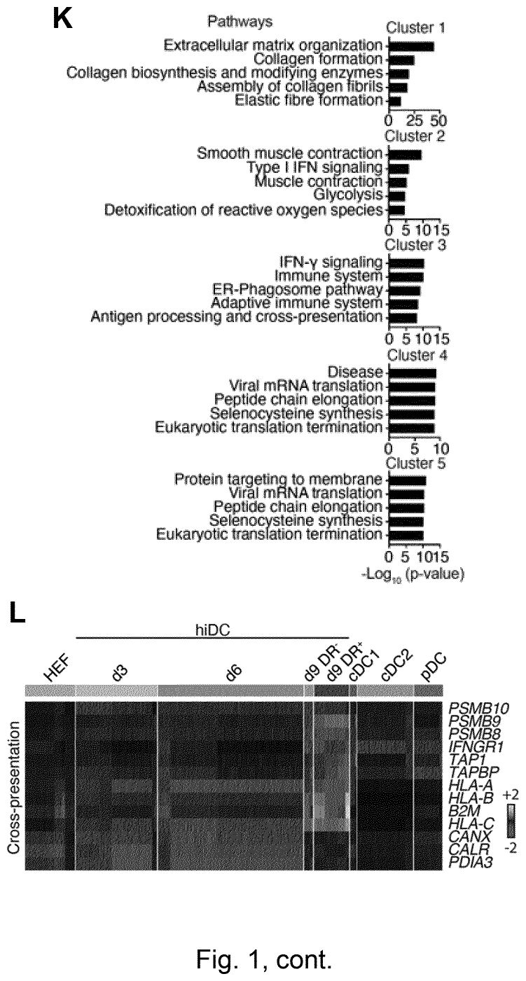

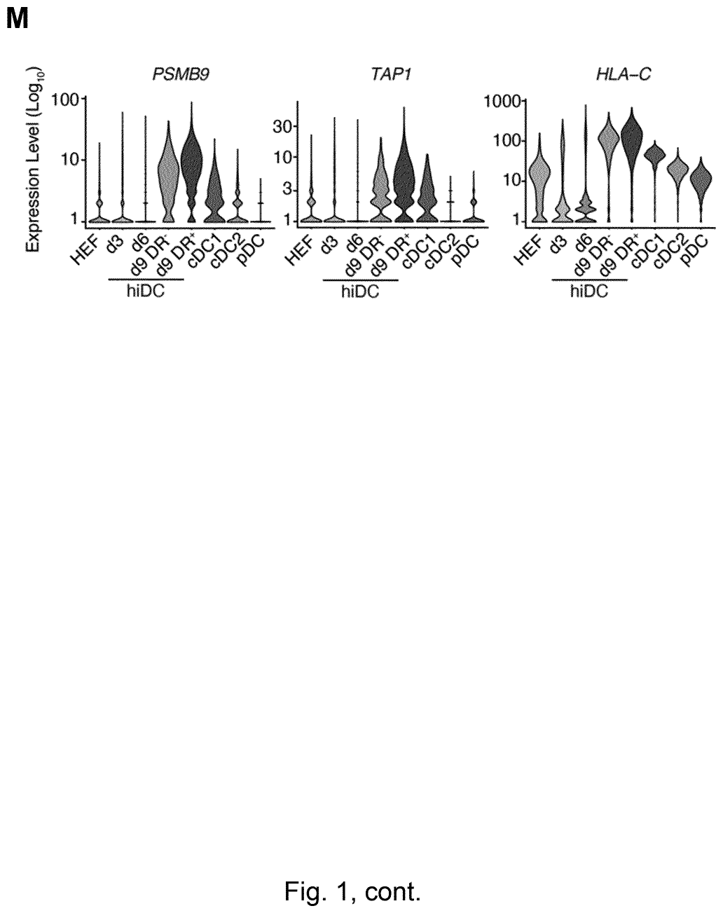

To characterize DC reprogramming of human fibroblasts at the transcriptional level, profiling of non-transduced HEFs (d0), human iDCs (hiDCs) at day 3 (CD45⁺, d3), day 6 (CD45⁺, d6) and day 9 (CD45⁺ HLA-DR⁺, d9 DR⁺; CD45⁺ HLA-DR⁻, d9 DR⁻), and peripheral blood cDC1s, cDC2s and pDCs was performed by single cell RNA-seq using the 10× Chromium system (FIG. 1. A).

### Methods

### The Vector

To induce DC fate in HEFs, a polycistronic construct encoding PIB (PU1, IRF8 and BATF3) separated by 2A sequences was cloned in a Doxycycline (Dox)-inducible lentiviral vector (tetO-PIB) and introduced to the cells (Rosa et al., 2018) (FIG. 1A).

### Single-Cell RNA Sequencing

Transduced and untransduced HEF cells were FACS sorted for scRNA-seq. Purified cells were loaded on a 10× Chromium (10× Genomics) according to manufacturer's protocol. scRNA-seq libraries were prepared using Chromium Single Cell 3′ v2 Reagent Kit (10× Genomics) according to manufacturer's protocol. Indexed sequencing libraries were constructed using the reagents from the Chromium Single Cell 3′ v2 Reagent Kit. Library quantification and quality assessment was determined using Agilent Bioanalyzer using the High Sensitivity DNA analysis kit. Indexed libraries were pooled in equal moles and sequenced on an Illumina NextSeq 500 using paired-end 26×98 bp sequencing mode. Coverage of approximately 100,000 reads per single cell was obtained.

### Scanning Electron Microscopy (SEM)

HEFs transduced with PIB factors were sorted (CD45⁺HLA-DR⁺) at day 8, plated in 0.1% gelatin-coated coverslips and analyzed at day 9 along with M2rtTA-transduced HEFs. Samples were washed in 0.1M Sorensen's phosphate buffer and fixed with 0.1M Sorensen s phosphate buffer pH 7.4, 1.5% formaldehyde and 2% glutaraldehyde at room temperature for 30 min. After fixation, samples were washed in 0.1M Sorensen's buffer. Samples were then dehydrated in a graded series of ethanol (50%, 70%, 80%, 90% and twice in 100%), critical point dried and mounted on 12.5 mm aluminum stubs. Samples were then sputtered with 10 nm Au/Pd (80/20) in a Quorum Q150T ES turbo pumped sputter coater and examined in a Jeol JSM-7800F FEG-SEM.

### DC Subset Classification

A scPred library (Alquicira-Hernandez et al., 2019) and publicly available DC single-cell expression data (Villani et al., 2017) was used for subset affiliation. In order to train classifier using scPred method (implemented as R library), the default parameters for getFeatureSpace, trainModel, was used as defined in tool vignette. To predict the assignment of DCs isolated from PBMCs to publicly availably DC subsets, the scPredict function was used with default parameters. For classification of hiDCs, the scPredict function was used with threshold=0.99 separately for each donor and then combined the number of cells affiliated to each subset.

### Results

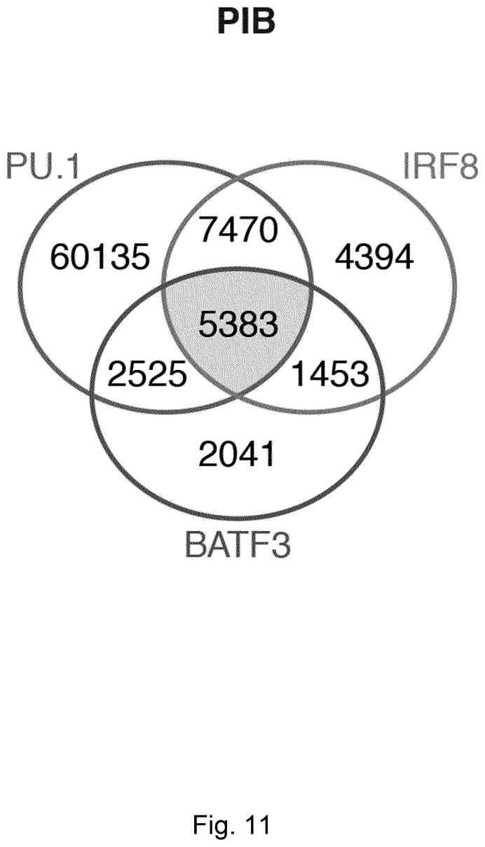

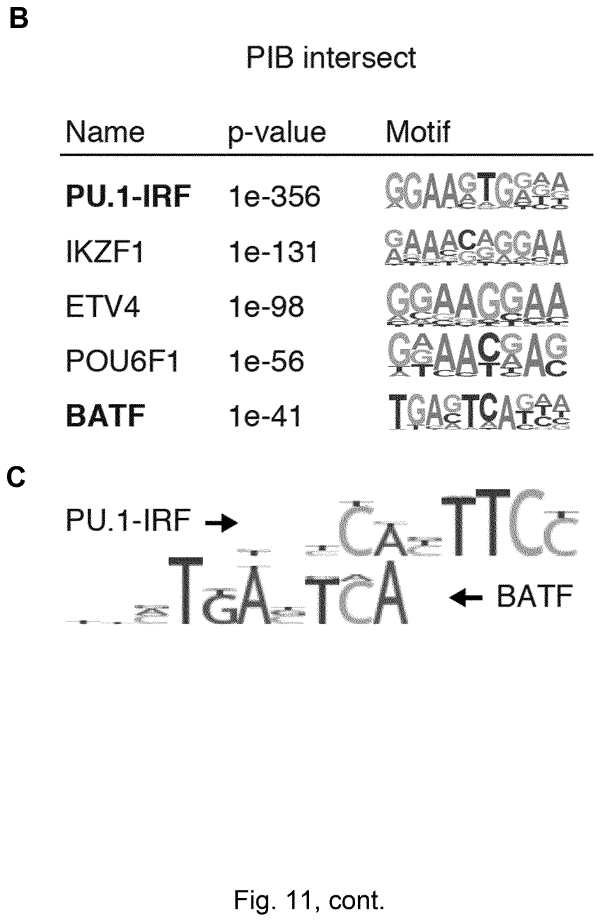

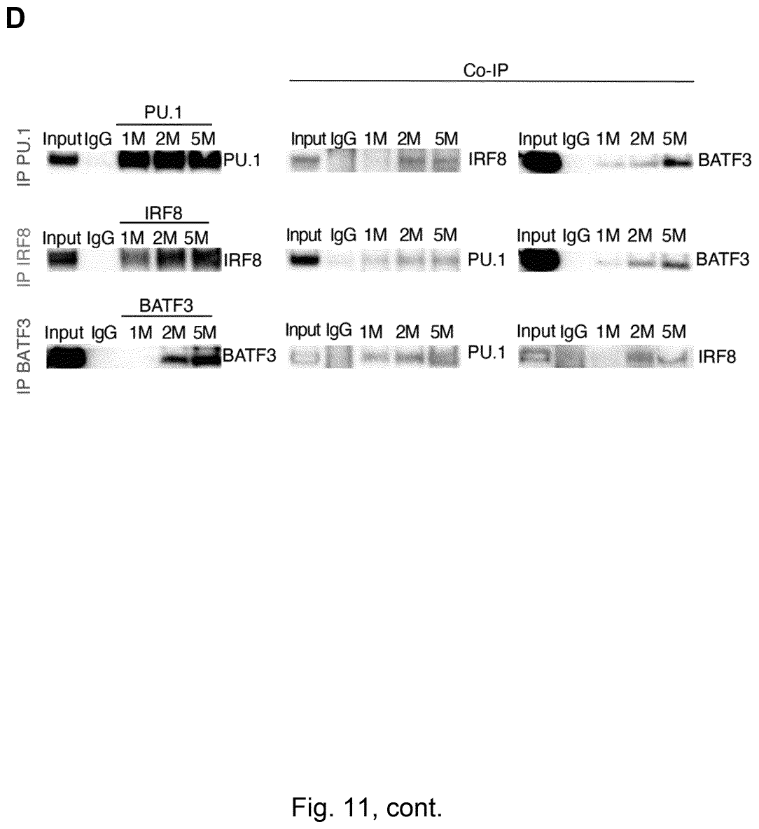

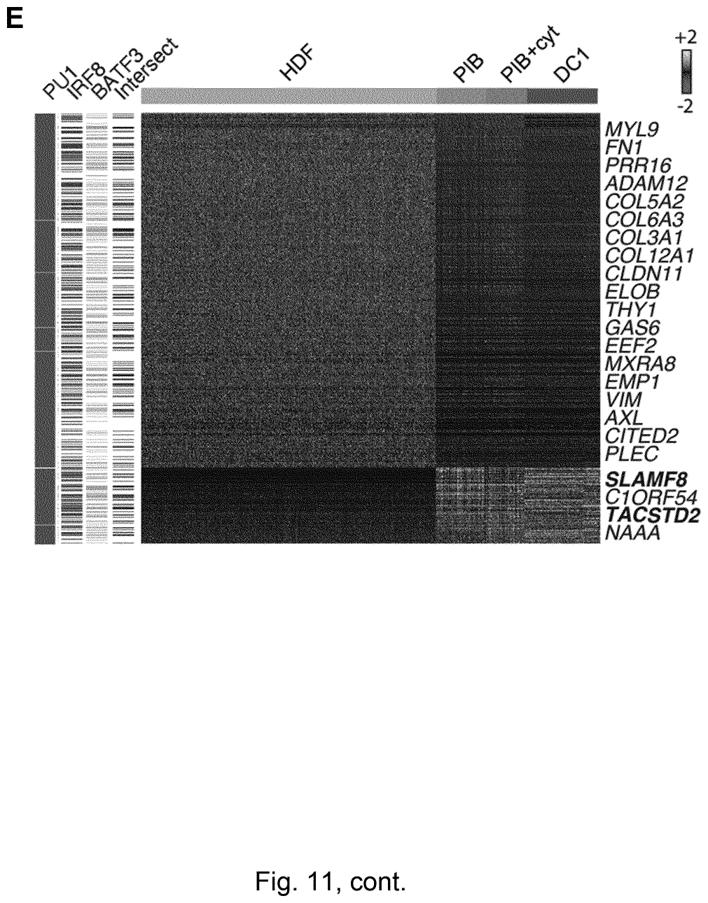

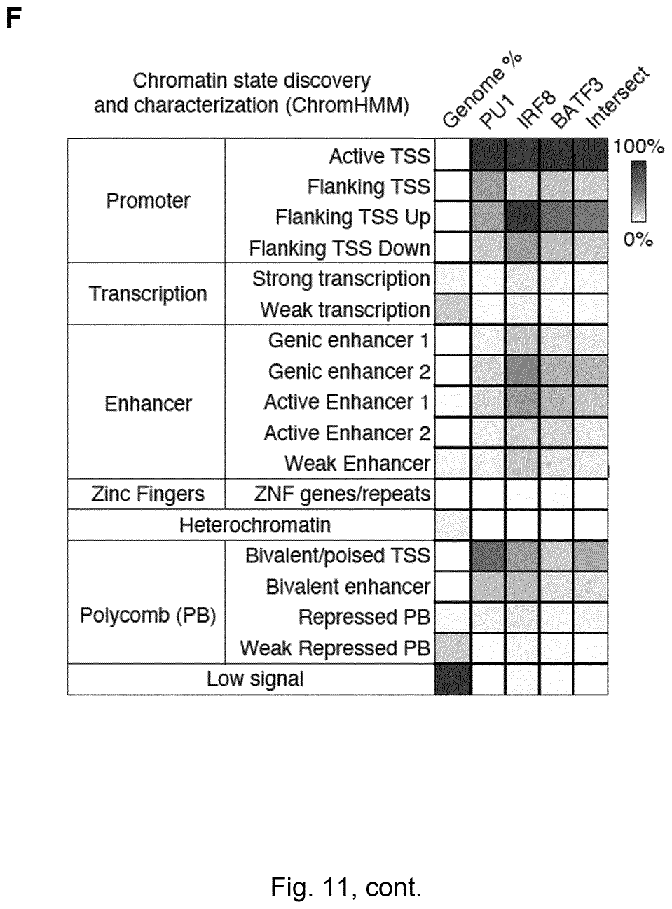

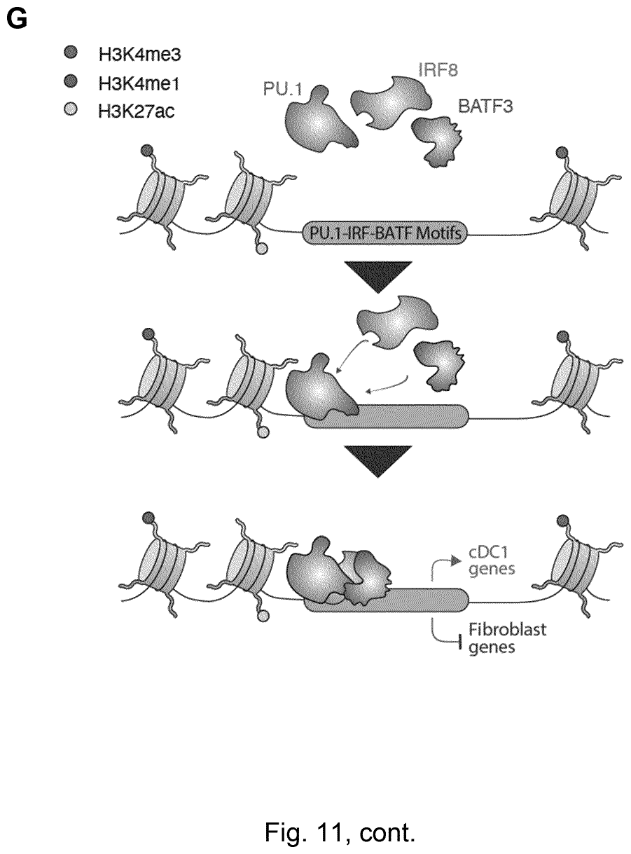

To induce DC fate in Human Embryonic Fibroblasts (HEFs), a polycistronic construct encoding PU.1, IRF8 and BATF3 separated by 2A sequences (PIB), cloned in a Doxycycline (Dox)-inducible lentiviral vector (TetO-PIB) (Rosa et al. 2018) was used (FIG. 1A). After transducing HEFs with PIB, the emergence of a CD45⁺ cell population at day 3 and a small population of CD45⁺ HLA-DR⁺ cells from day 6 to day 9 with DC-like morphology was observed (FIG. 1B-D), which was named human induced DCs (hiDCs). To elucidate transcriptional changes, scRNA-seq was performed using the 10× Chromium system. 45,870 cells were profiled from 3 donors, including peripheral blood cDC1s, cDC2s, pDCs, non-transduced HEFs (d0), hiDCs at day 3 (CD45⁺, d3), day 6 (CD45⁺, d6) and day 9 (CD45⁺HLA-DR⁻, d9 DR⁻; CD45⁺HLA-DR⁺, d9 DR⁺). t-Distributed stochastic neighbor embedding (t-SNE) visualization of the dataset highlighted four clusters: HEF, cDC1, cDC2 and pDC (FIG. 1E). While hiDC d3 and d6 did not map specifically to any clusters, hiDCs d9 mapped with cDC1s, with DR⁺ being closer to cDC1s than DR⁻. These data suggest human cDC1 reprogramming requires a timeframe of 9 days and CD45⁺HLA-DR⁻ cells represent a partial reprogrammed cell state. Next, the single cell data obtained was integrated with a publicly available DC datasets (Villani et al. 2017) using scPred (Alquicira-Hernandez et al. 2019). As expected, it was observed that 53.8% of purified cDC1s were assigned to the cDC1 subset, 66.9% and 29.8% of cDC2s to the DC2 and DC3 subsets, respectively, and 66.5% of pDCs to the DC6 subset (fig. S3). While HEFs were unaffiliated, 1.3% of d3, 5.3% of d6, 14.4% of d9 DR- and 36.7% of d9 DR⁺ hiDCs were assigned specifically to the cDC1 subset, suggesting that PU1, IRF8 and BATF3 progressively impose cDC1 signatures with a degree of heterogeneity that does not cross subset boundaries (FIG. 1F-G). cDC1-affiliated cells express higher levels of the cDC1-specific genes CADM1 and WDFY4 when compared to their unaffiliated counterparts (Villani et al. 2017) (FIG. 1H). Next, the most variable genes across the dataset were extracted and grouped in 5 clusters (FIG. 11). Cluster 1 contains genes highly expressed in HEFs and silenced during reprogramming. Cluster 2 highlights early transcriptional changes during reprogramming and cluster 3 includes the cDC1-specific genes C1orf54, ANPEP, TACSTD2 and SLAMF8 (Heidkamp et al. 2016; See et al. 2017; Villani et al. 2017) highly expressed in d9 hiDCs and cDC1s (FIG. 1J, table 2). Cluster 4 and cluster 5 contain genes highly expressed in cDC2s and pDCs, respectively. Interestingly, d9 hiDCs express high levels of antigen processing and cross-presentation genes including PSMB9, TAP1 and HLA-C (FIG. 1K-M), suggesting that reprogrammed cells have acquired cross-presentation capacity.

TABLE 2
Genes included in cluster 3 from FIG. 11.

| | | | | | |
|---|---|---|---|---|---|
| CST3 | FOS | DBNDD2 | FUT8 | UBXN1 | PAK1 |
| TACSTD2 | ATP5IF1 | QPCT | CTSZ | NIBAN1 | FAM49B |
| CD40 | HMOX1 | NFE2L3 | GM2A | EVI2B | ARPC2 |
| VMO1 | PRKAR2B | ITGB7 | NABP1 | LIMA1 | ZFHX3 |
| CPVL | LMCD1 | PPP1R12B | REEP5 | VAMP5 | NOTCH2 |
| CD74 | HLA-DQB1 | RAB29 | GTF2B | ID2 | BLOC1S1 |
| NAAA | GYPC | RAB8B | CD226 | NEAT1 | MGAT1 |
| DNASE1L3 | MTPN | SUB1 | HPS5 | SAA1 | KIAA1324 |
| UCP2 | MARCKSL1 | SLC31A2 | LGMN | GPX3 | STOM |
| HLA-DRB1 | C1orf54 | LPGAT1 | TANK | TNFSF10 | BCL6 |
| RGCC | FNBP1 | VRK2 | TNFAIP8 | IRF1 | PKN2 |
| HLA-C | SNX3 | IQGAP1 | NRBF2 | DSE | AKR1A1 |
| TXN | SLAMF8 | PSME2 | WDFY4 | RAB7A | PTPRE |
| GRN | BIRC3 | M6PR | MAP3K13 | ATG3 | PRSS3 |
| GSTM4 | CCND1 | SPI1 | CORO1B | HLA-E | HLA-F |
| SAT1 | STX12 | PREX1 | CBL | G3BP2 | SMCO4 |
| CPNE3 | FKBP1B | HCK | PSMB8 | HSD17B14 | TMSB4X |
| FCGRT | PFDN2 | PLEKHO1 | BID | RGS10 | ANPEP |
| HLA-B | TPMT | OAZ2 | MLEC | ZNF710 | IFI16 |
| HLA-DMA | PPA1 | MEA1 | TPM3 | PLEK | RAB32 |
| HLA-DPA1 | ATOX1 | GTF2F2 | C20orf27 | F11R | |
| HLA-A | CCPG1 | HLA-DPB1 | CREB3 | CXCL16 | |
| PPT1 | PSMB5 | PMM1 | PSMB9 | UBL7 | |
| HLA-DRA | CTTNBP2NL | ACTR3 | SHTN1 | FUCA1 | |

### CONCLUSION

Together, these data demonstrate that PIB factors impose a cDC1 signature in human fibroblasts.

### Example 3. Single Cell Analysis Highlights Pathways Associated with Successful and Unsuccessful cDC1 Reprogramming

It was hypothesized that single cell RNA-seq could be used to dissect human DC reprogramming trajectories and reveal pathways or factors correlated with successful reprogramming and therefore enable the optimization of cDC1 reprogramming in human cells.

### Methods

### Pseudo-Time Reconstruction

Monocle3 library (Cao et al. 2019) was used to order cells on a reprogramming pseudotime. Monocle3 was run on tSNE with following parameter: use_partition=FALSE that assume that all cells in the dataset descend from a common transcriptional ancestor. The root of the trajectory was selected automatically. In order to mitigate the batch effect in identification of genes that vary along a trajectory, batch correction was performed using regressBatches function from batchelor R library (http://bioconductor.org/packages/release/bioc/html/batchelor.html). Next, genes that vary over a trajectory were identified using graph_test function, and grouped into 21 distinct modules using find_gene_modules function and clustered using Ward.D2 method in pheatmap R library (https://cran.r-project.org/web/packages/pheatmap/index.html). The genes were defined as successful and unsuccessful reprogramming groups according to clustering results.

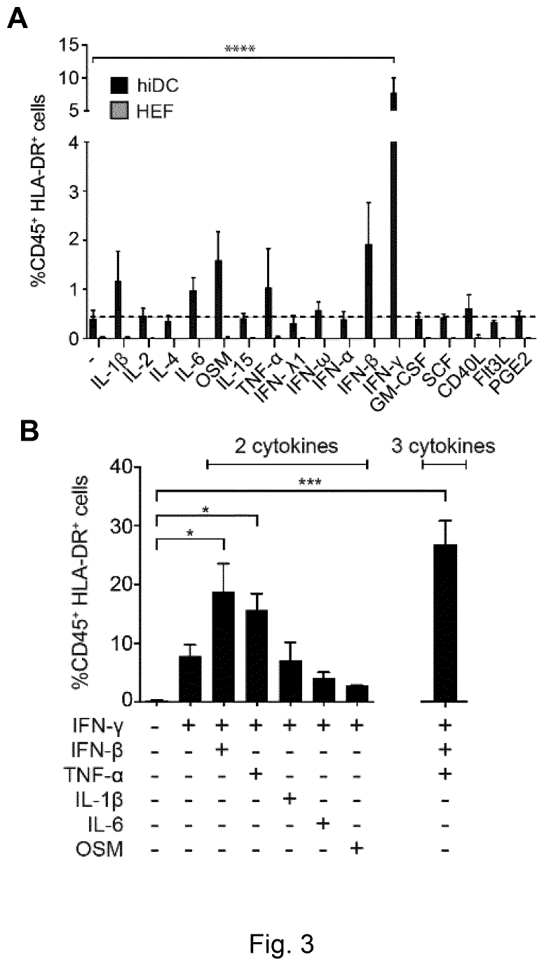

Next those gene lists were additionally filtered out by calculating means for each gene in each cell population and using the following criteria: for successful reprogramming—hiDC d9 DR⁺ (affiliated)>hiDC d9 DR⁺ (unaffiliated) & hiDC d9 DR⁻ (affiliated)>hiDC d9 DR⁺ (unaffiliated) & hiDC d9 DR⁺ (affiliated)>HEF & hiDC d9 DR⁻ (affiliated)>HEF and the reverse for all comparisons for unsuccessful reprogramming. To reconstruct reprogramming dynamics scVelo v0.2.4 (Bergen et al. 2020) was used. For these analyses, the sparse expression matrix generated by cellranger analysis pipeline was converted to spliced and unspliced matrix using Velocyto v0.17.17 (http://velocyto.org). scVelo was run with default settings. Additionally, scVelo was also used to recover the latent time, selected top 1000 genes that are changing along the latent time and visualized them on a heat map. For additional samples (FIG. 3), Monocle3 library was used to order cells on a pseudo-time course during HEF to hiDC reprogramming using the same approach.

### Transcription Factor (TF) Co-Regulatory Network Analysis

In order to construct TF networks, genes associated with successful or unsuccessful cDC1 reprogramming were submitted for analysis using ChEA3 (https://maayanlab.cloud/chea3/) and integrated by average rank across libraries. TF network was visualized as network plot with points representing human TFs based on their co-expression similarity.

### Results

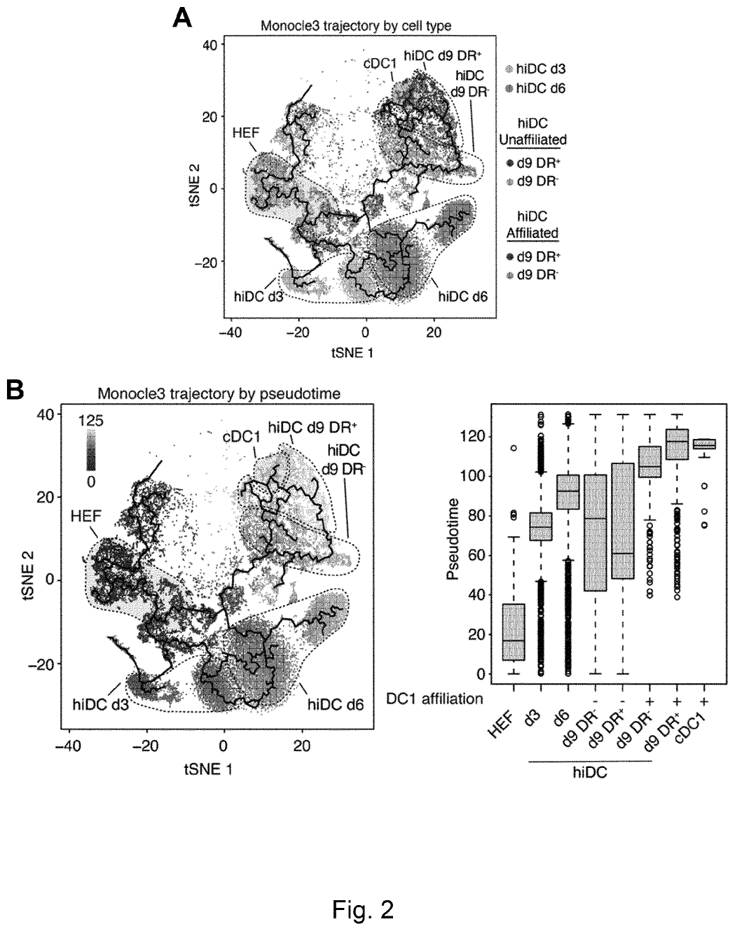

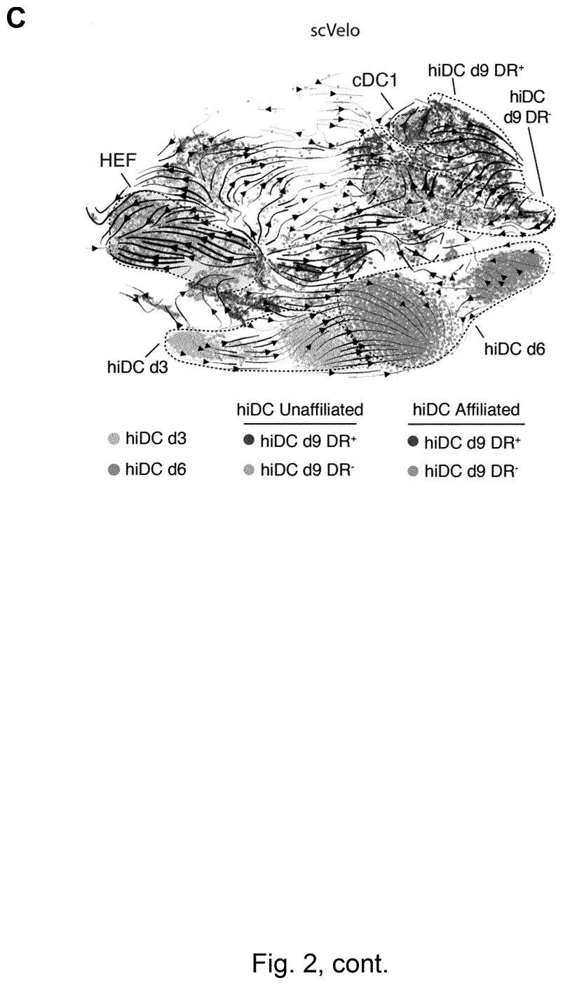

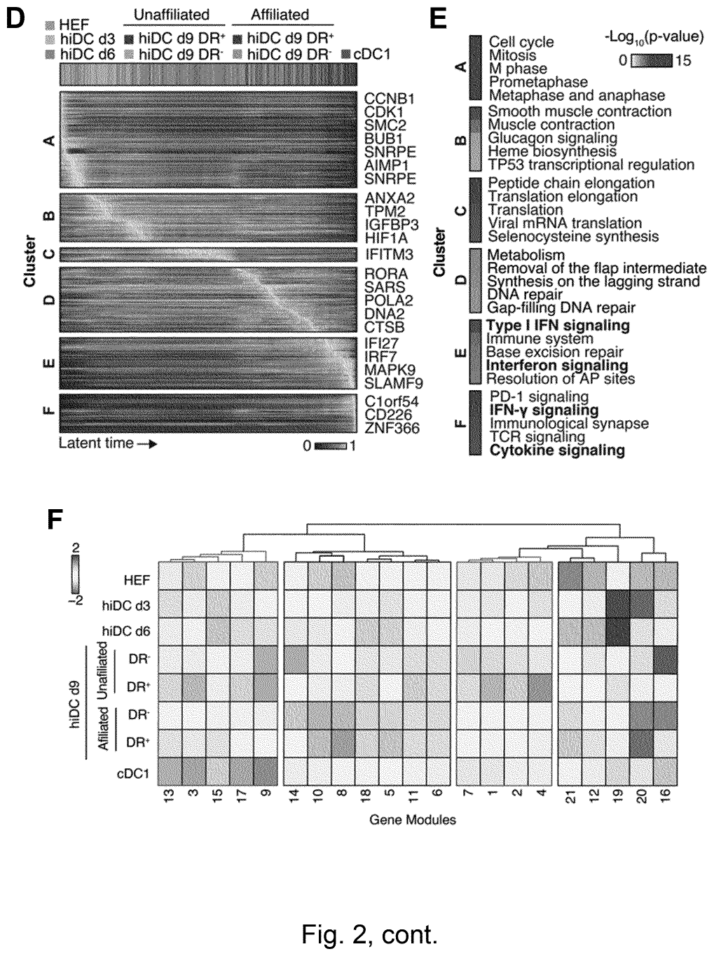

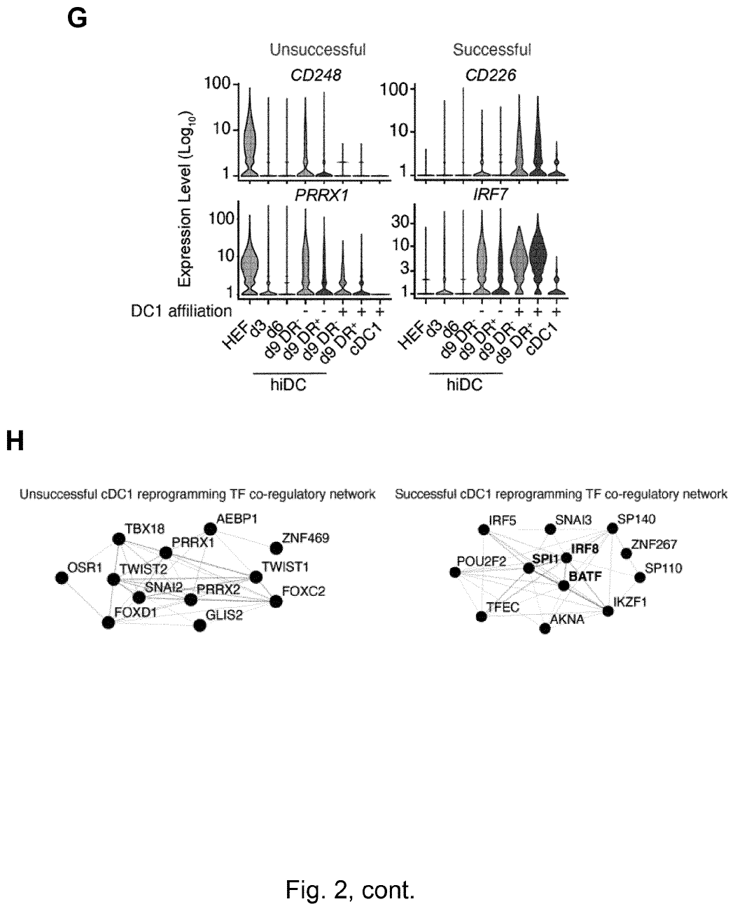

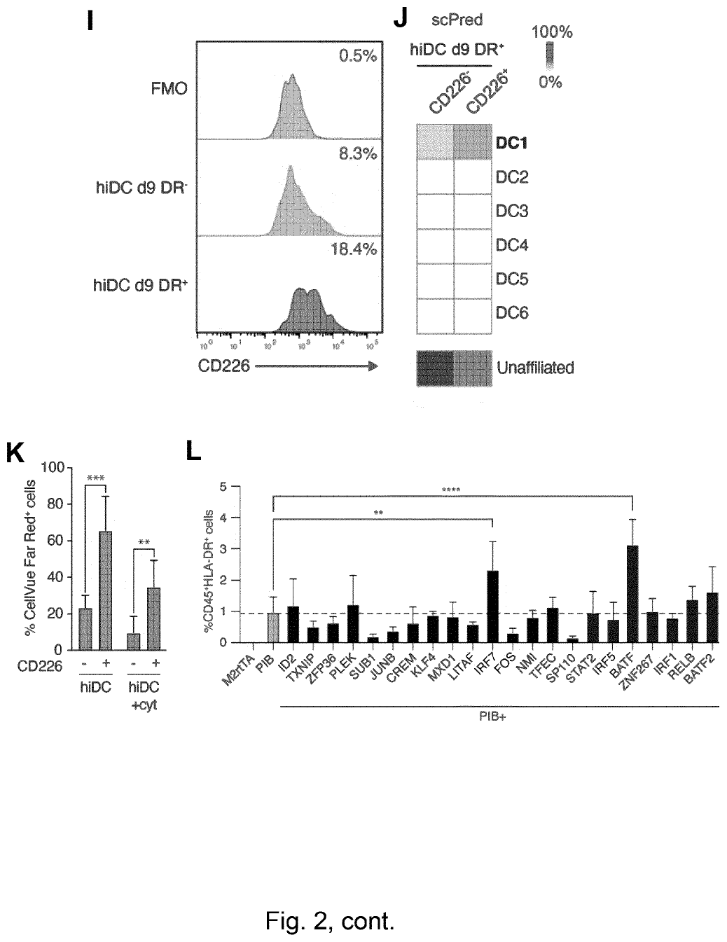

The inventors used Monocle 3 to reconstruct the cDC1 reprogramming trajectory (Cao et al. 2019). HEFs and cDC1s were placed in the beginning and end of pseudotime, respectively (FIG. 2A-B). While d9 hiDCs were placed at the end of the trajectory with cDC1s, d3 and d6 hiDCs were located in the middle highlighting the stepwise transition of single cell transcriptomes during cDC1 reprogramming. Importantly, affiliated d9 hiDCs were positioned later in pseudotime when compared to their unaffiliated counterparts, suggesting that trajectory reconstruction is capturing a successful cDC1 reprogramming path. A “dead-end” trajectory with unaffiliated hiDCs mapping closer to HEFs was also observed, suggesting that these cells fail to enter the successful reprogramming path. To infer expression dynamics and directionality of individual cells during reprogramming, scVelo analysis (Bergen et al. 2020) was applied to the reprogramming trajectory. hiDC velocities are mainly aligned with the cDC1 reprogramming trajectory projected by Monocle 3 (FIG. 2C). Nevertheless, hiDCs mapping closer to dead-end trajectory display velocities pointing towards HEFs were observed, in agreement with previous reports describing unsuccessful reprogramming paths marked by the expression of genes associated with the original cell state (Biddy et al. 2018; Zhou et al. 2019; Treutlein et al. 2016). Reconstruction of gene expression dynamics using latent time showed downregulation of cell cycle genes during cDC1 reprogramming (FIG. 2D-E), suggesting that hiDCs exit cycle. The inventors also observed enrichment in cytokine and IFN type-I and II (IFN-γ) signaling pathways later in latent time. The downregulation of cell cycle and upregulation of IFN gene signatures go in line with our previous findings in the mouse system (Rosa et al. 2018), suggesting a species-conserved role for IFN signaling in cDC1 reprogramming. To map stage-specific gene changes along reprogramming, the inventors clustered genes differentially expressed along pseudotime in 21 modules (FIG. 2F). Unaffiliated hiDCs fail to downregulate several gene modules enriched in HEFs, including modules 1, 2, 4 and 7 (unsuccessful reprogramming). Affiliated hiDCs and cDC1 are enriched in genes expressed in modules 3, 9, 13, 15 and 17 (successful reprogramming). Extraction of genes encoding surface molecules and transcriptional regulators from these modules highlighted fibroblast genes enriched in unsuccessful reprogramming (CD248 and PRRX1) and DC genes upregulated in successful reprogrammed cells, including the cDC1 marker CD226 (Heidkamp et al. 2016) and IRF7 (Honda et al. 2005) (FIG. 2G, Table 1). Transcription factor enrichment analysis for unsuccessful reprogramming genes identified previously described barriers to direct reprogramming, including TWIST1, TWIST2, PRRX1, PRRX2 and OSR1 (Tomaru et al. 2014) (FIG. 2H). Interestingly, the same analysis on successful reprogramming genes reinforced the importance of PU1, IRF8 and BATF in the establishment of successful cDC1 reprogramming gene signatures.

### CONCLUSION

These data demonstrate that enforced expression of the transcription factors PU.1, IRF8 and BATF3 may improve reprogramming efficiency and highlights a role for cytokine and IFN signaling in cDC1 reprogramming.

### Example 4. Single Cell Analysis Highlights Surface Markers Associated with Successful Reprogramming Enabling the Identification and Prospective Isolation of Successfully Reprogrammed hiDCs

The inventors hypothesized that surface markers enriched in successful cDC1 reprogramming (Table 1) would allow the identification and prospective isolation of successfully reprogrammed hiDCs. As a proof of concept, one of these surface markers—CD226 was selected, CD226⁺ reprogrammed hiDCs were purified and their cDC1 identity was compared to CD226⁻ reprogrammed hiDCs.

### Methods

First, the inventors evaluated surface expression of CD226 in partially-reprogrammed CD45⁺ HLA-DR⁺ and reprogrammed CD45⁺ HLA-DR⁺ hiDCs. Then, CD45⁺ HLA-DR⁺CD226⁺ hiDCs were purified and their cDC1 identity was compared to that of CD45⁺HLA-DR⁺CD226⁻ hiDCs using the scPred system. See previous Examples for experimental details.

### Results

First, it was observed that CD45⁺HLA-DR⁺ hiDC expressed higher levels of CD226 than CD45⁺HLA-DR⁻ hiDC (FIG. 2I). Then, to validate the utility of CD226 to identify cDC1-like cells, CD45⁺HLA-DR⁺CD226⁺ hiDC were purified and profiled by scRNA-seq. Interestingly, CD226⁺ cells showed increased cDC1 affiliation (from 19.5% to 40.9%) (FIG. 2J). In addition, it was observed that CD226⁺ hiDCs performed better in dead cell phagocytosis when compared to CD226⁻ hiDCs, suggesting that CD226 marks functional hiDCs (FIG. 2K).

### CONCLUSION

These data suggest that surface markers associated with successful reprogramming, including CD226, allow the isolation of more functional hiDCs with refined cDC1 identity.

### Example 5. Single Cell Analysis Identifies Transcription Factors Associated with Successful cDC1 Reprogramming Able to Cooperate with PU.1, IRF8 and BATF3 and Increase cDC1 Reprogramming Efficiency

The inventors hypothesized that transcription factors enriched in successful cDC1 reprogramming (Table 1) could cooperate with PU.1, IRF8 and BATF3 to increase cDC1 reprogramming efficiency. As a proof of concept, 22 transcription factors were selected (ID2, TXNIP, ZFP36, PLEK, SUB1, JUNB, CREM, KLF4, MXD1, LITAF, IRF7, FOS, NMI, TFEC, SP110, IRF5, STAT2, BATF, ZNF267, IRF1, RELB and BATF2) which associated with successful cDC1 reprogramming or predicted to regulate successful cDC1 reprogramming gene signatures (see Transcription factor (TF) co-regulatory network analysis described in Example 3) and their ability to increase cDC1 reprogramming efficiency when co-expressed with PU.1, IRF8 and BATF3 was evaluated.

### Methods

Coding regions of ID2, TXNIP, ZFP36, PLEK, SUB1, JUNB, CREM, KLF4, MXD1, LITAF, IRF7, FOS, NMI, TFEC, SP110, IRF5, STAT2, BATF, ZNF267, IRF1, RELB and BATF2 were individually cloned in the pFUW-TetO plasmid. Lentiviral particles encoding each individual transcription factor, or PU1, IRF8 and BATF3 (pFUW-tetO-PIB), or the reverse tetracycline transactivator M2rtTA under the control of constitutively active human ubiquitin C promoter (pFUW-UbC-M2rtTA) were used for co-transduction (Rosa et al. 2018). Reprogramming efficiency was evaluated by flow cytometry in HEFs 9 days after transcription factor overexpression.

### Results

To assess whether additional regulators would enhance reprogramming, the inventors supplemented PU1, IRF8 and BATF3 with individual transcription factors associated with successful cDC1 reprogramming and observed that IRF7 and BATF increased cDC1 reprogramming efficiency (FIG. 2L). IRF7 is a transcriptional regulator downstream inflammatory signalling (Honda et al. 2005). BATF is highly homologous to BATF3 and was shown to compensate BATF3 during cDC1 development (Tussiwand et al. 2012).

### CONCLUSION

These data suggest that transcription factors associated with successful reprogramming, including IRF7 and BATF, can increase cDC1 reprogramming efficiency.

### Example 6. Increased cDC1 Reprogramming Efficiency Using External Cytokines

Motivated by the enrichment of cytokine signalling in successfully reprogrammed hiDCs and their role in human DC specification, the inventors hypothesized that cytokines could synergize with PU.1, IRF8 and BATF3 in DC reprogramming.

### Methods

17 human hematopoietic cytokines including inflammatory mediators (Table 3) were individually added to the culture media 2 days after PIB induction in HEFs, and reprogramming efficiency was measured at day 9. Changes in reprogramming efficiency were analysed using both single cytokines and combinations of two or three cytokines.

TABLE 3
List of the 17 human hematopoietic cytokines
used in the current experiment.

| Cytokine | Concentration (ng/ml) |
|---|---|
| CD40I | 1000 |
| Flt3I | 40 |
| GM-CSF | 100 |
| IFN-α | 100 |
| IFN-β | 20 to 100 |
| IFN-γ | 1 to 100 |
| IFN-λ1 | 100 |
| IFN-ω | 100 |
| IL-1β | 25 |
| IL-2 | 25 |
| IL-4 | 25 |
| IL-6 | 20 |
| IL-15 | 200 |
| Oncostatin M | 25 |
| Prostaglandin 2 | 1000 |
| SCF | 50 |
| TNFα | 20 to 25 |

### Results

IFN-γ had the most significant impact promoting a 20-fold increase in CD45⁺ HLA-DR⁺ cell generation (7.9%±2.2% versus 0.4%±0.2% without cytokines) (FIG. 3A). Other inflammatory cytokines also increased reprogramming efficiency, including IL-1β (3-fold), IL-6 (2.5-fold), Oncostatin M (4-fold), TNF-α (3-fold) and IFN-β (4-fold). FLT3L, IL-4 and GM-CSF used for in vitro differentiation of DCs from progenitors (Balan et al., 2018) or monocytes (Chapuis et al., 1997) did not impact reprogramming efficiency. Combining IFN-γ with IFN-β or TNF-α induced an additional 2.5- and 2-fold increase in reprogramming efficiency, respectively. Combining the three cytokines, IFN-γ, IFN-β and TNF-α, resulted in 28.1%±3.4% hiDCs, a 70-fold increase when compared to the condition without cytokines (FIG. 3B).

### CONCLUSION

These data strongly suggests that cDC1 reprogramming efficiency is increased with the provision of inflammatory cytokines.

### Example 7. Increased cDC1 Reprogramming Efficiency Using Stronger Constitutive Promoters

Given that trajectory reconstruction of cDC1 reprogramming correlated PU.1, IRF8 and BATF3 expression with the successful establishment of cDC1 fate, the inventors asked whether forced expression of reprogramming factors using a stronger constitutive promoter would increase reprogramming efficiency.

### Methods

The PIB polycistronic cassette followed by IRES-GFP was cloned into constitutive vectors utilizing multiple promoters on lentiviral backbones and DC reprogramming efficiency in MEFs harbouring the Clec9a-tdTomato reporter (Rosa et al., 2018) was evaluated. The following vector backbones and promoters were used: pFUW-UbC, pRRL.PPT-SFFV, pRRL.PPT-PGK, pRRL.PPT-EF1S, pHAGE2-EF1 and pWPXL-EF1i.

### Results

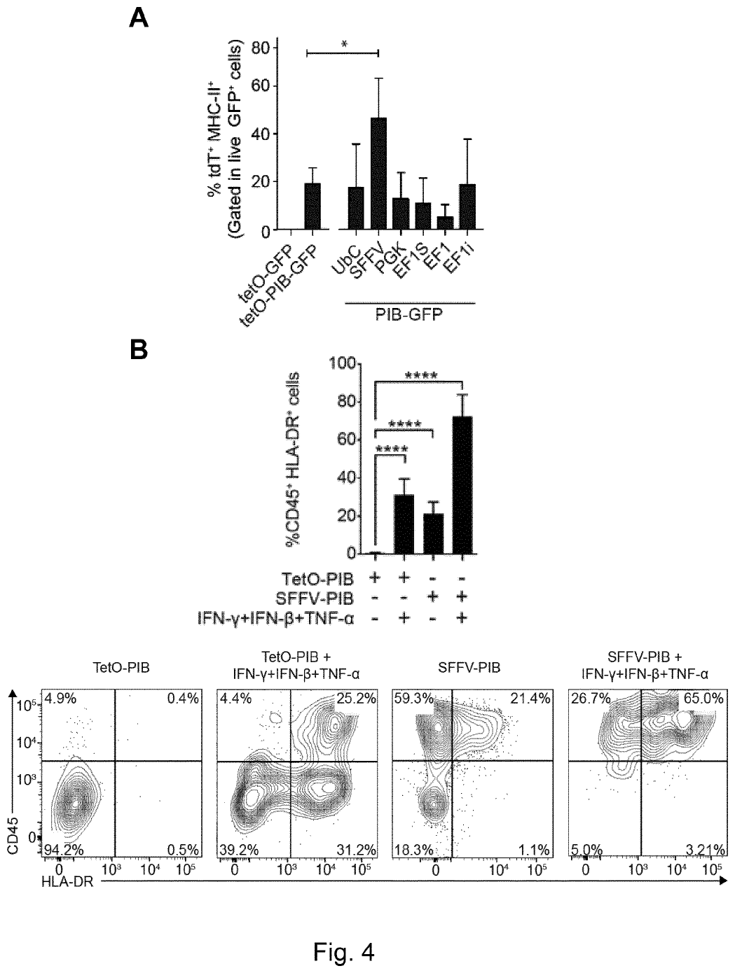

PIB overexpression driven by SFFV promoter induced superior efficiency (46.6%±16.7% tdTomato⁺ MHC-II⁺ cells) in mouse cells (FIG. 4A). In HEFs, the emergence of 21.3±6.1% hiDCs was observed with the SFFV system (FIG. 4B). Moreover, constitutive overexpression of PIB induced surface expression of CD45 in the majority of fibroblasts, suggesting that a bigger cohort of cells start the DC reprogramming process when compared to the Dox-inducible system (FIG. 4B).

### CONCLUSION

These data indicates that the lentiviral vector with the SFFV promoter can be used to improve reprogramming in human cells.

### Example 8. Increased cDC1 Reprogramming Efficiency Using a Combination of Cytokines and Stronger Constitutive Promoters

Given that IFN-γ, IFN-β and TNF-α signaling and SFFV-mediated constitutive overexpression of PU.1, IRF8 and BATF3 increased cDC1 reprogramming efficiency, the inventors asked whether inflammatory cytokine signaling would synergize with constitutive overexpression of the reprogramming factors to achieve higher reprogramming efficiency.

### Method

The impact on reprogramming efficiency after a combination of cytokine treatment and SFFV-driven PIB induction was evaluated. The scPred system was used for integration with “natural” DCs. See previous Examples for experimental details.

### Results

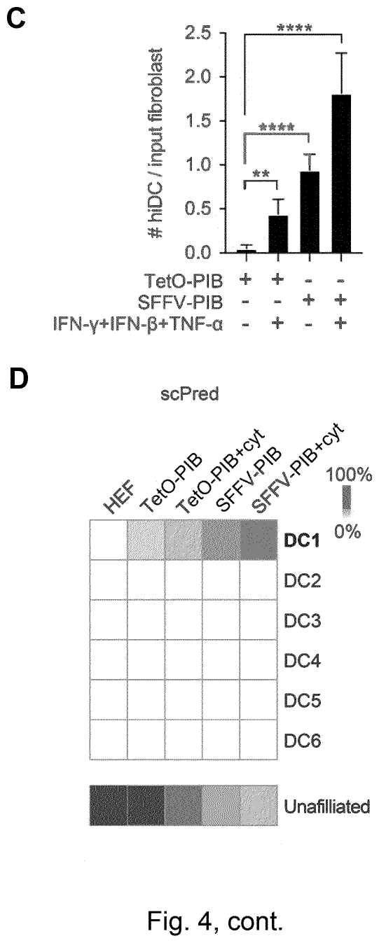

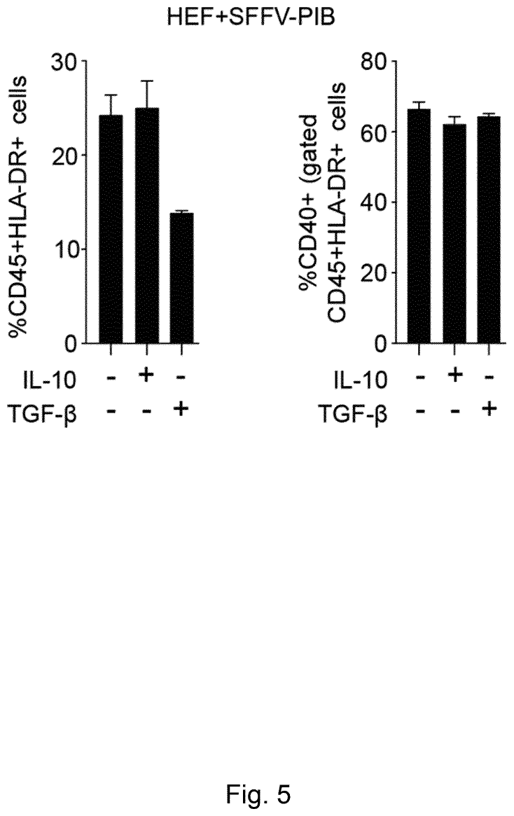

A combination of SFFV-driven induction and treatment with IFN-γ, IFN-β and TNF-α, yielded a 76.9±11.9% of hiDCs, which was a 190-fold increase in cDC1 reprogramming efficiency compared to the original protocol (FIG. 4B). Moreover, an increase in the absolute numbers of hiDCs generated was observed with the improved protocol (FIG. 4C). To characterize the cDC1 identity of hiDCs generated with the improved reprogramming protocol, hiDCs obtained with inducible (tetO-PIB) and constitutive (SFFV-PIB) systems with and without cytokines were profiled and scPred was used for integration with peripheral blood DCs (FIG. 4E). Interestingly, 61.4% and 53.2% of hiDCs generated with SFFV were affiliated to cDC1 lineage, with and without cytokines respectively, in contrast to only 33.4% and 22.0% generated with the inducible system (tetO-PIB). These data suggest that cytokine signalling and enforced expression of PU.1, IRF8 and BATF3 synergize for the successful establishment of a cDC1-like cell fate. IL-10 did not impact reprogramming efficiency and TGF-β reduced it 2-fold (FIG. 5). IL-10 and TGF-β signalling did not impact CD40 expression in hiDCs.

### CONCLUSION

These data suggest that 1) the improved protocol increased both cDC1 reprogramming efficiency and cDC1 identity, and 2) cytokines and enforced expression of PU1, IRF8 and BATF3 synergize for successful reprogramming.

### Example 9. Functional Reprogramming of Human cDC1-Like Cells

cDC1s orchestrate adaptive immunity by multiple mechanisms including secretion of cytokines and antigen presentation to T cells. Motivated by the induction of a cDC1-like gene expression profile in human fibroblasts after PU.1, IRF8 and BATF3 overexpression, the inventors asked whether hiDCs could function as naturally-occurring cDC1s.

### Methods

In order to investigate whether the hiDCs share the same functionality as naturally occurring DCs, the hiDCs were challenged with toll-like receptor 4 (TLR4) [Lipopolysaccharide (LPS)], TLR3 [Polyinosinic-polycytidylic acid (Poly I:C)] or TLR7/8 [Resiquimod (R848)], or all TLR combined. The surface expression of the co-stimulatory molecules CD40 and CD80 (required for T cell activation) was analysed with flow cytometry and used as a marker for T cell activation.

To access dead cell phagocytosis, HEK293T cells were exposed to ultraviolet (UV) irradiation (50 J/m2) to induce cell death and labeled with CellVue Claret Far Red Fluorescent Cell Linker Kit (Sigma). hiDCs at day 9, HEFs and cDC1s were incubated with far red-labeled dead cells for 2 hours, washed with PBS 5% FBS, and analyzed in BD LSRFortessa X-20. Dead cell incorporation was quantified in live CD45⁺ HLA-DR⁺ hiDCs, CD45⁺ HLA-DR⁺CD226⁻ hiDCs, CD45⁺ HLA-DR⁺CD226⁺ hiDCs, CD141⁺CLEC9A⁺ peripheral blood cDC1s or in control populations using the far-red channel. For time-lapse fluorescent microscopy imaging of dead cell phagocytosis, far red-labeled dead cells were added to FACS-sorted HEF-derived CD45⁺ HLA-DR⁺ hiDC cultures immediately before starting image acquisition on a Zeiss Celldiscoverer 7. Microscopy images were taken every 10 minutes for 16 hours.

To access inflammatory cytokine secretion, levels of human cytokines were quantified in 25 μl of culture supernatants of FACS-sorted CD45⁺HLA-DR⁺ day 9 hiDCs generated in the presence or absence of cytokines, HEFs cultured in the presence or absence of cytokines, moDCs and FACS-sorted CD141⁺CLEC9A⁺ XCR1⁺cDC1s using cytometric bead array kits (LEGENDplex Human Anti-Virus Response Panel, BioLegend), according to the manufacturer's instructions. When stated, LPS, Poly I:C or R848, or the three combined stimuli were added overnight before analysis. Acquisition was performed in a FACSCanto, and data were analyzed using LEGENDplex software (BioLegend).

To access cross-presentation ability, HEFs, moDCs, magnetic-activated cell sorting (MACS)-enriched Clec9a⁺cDC1s and hiDCs at reprogramming day 8 were stimulated with LPS (3 ng/ml), Poly I:C (25 μg/ml) and R848 (3 ng/ml). After overnight stimulation, cells were washed in PBS containing 2% FBS and pulsed with 2 μl/ml of CMV protein (Miltenyi Biotec). After 3 hours, cells were washed and co-cultured with MACS-enriched CD8⁺ T cells isolated from CMV-seropositive donors. CMV positivity was verified by flow cytometry using a CMV Dextramer (Immudex). 1×10⁵ CD8⁺ T cells and 5×10⁴ DCs were co-cultured in 96-well plates in 200 μl of X-VIVO 15. After 24 h, T-cell activation was measured by quantifying IFN-γ levels in the supernatants using ELISA (BD). Absorbance was read at 490 nm in a GloMax Discover Microplate Reader (Promega).

### Results

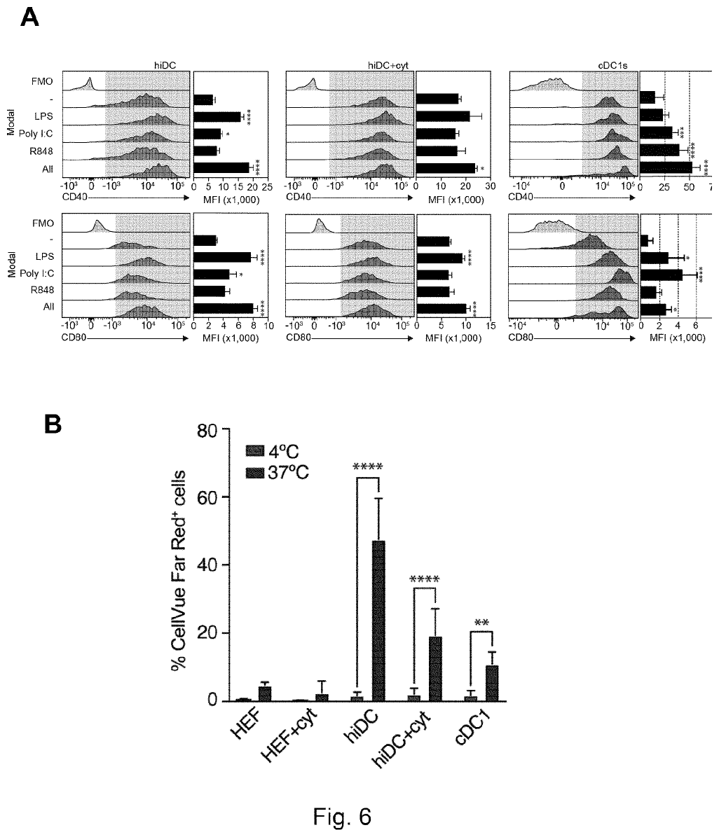

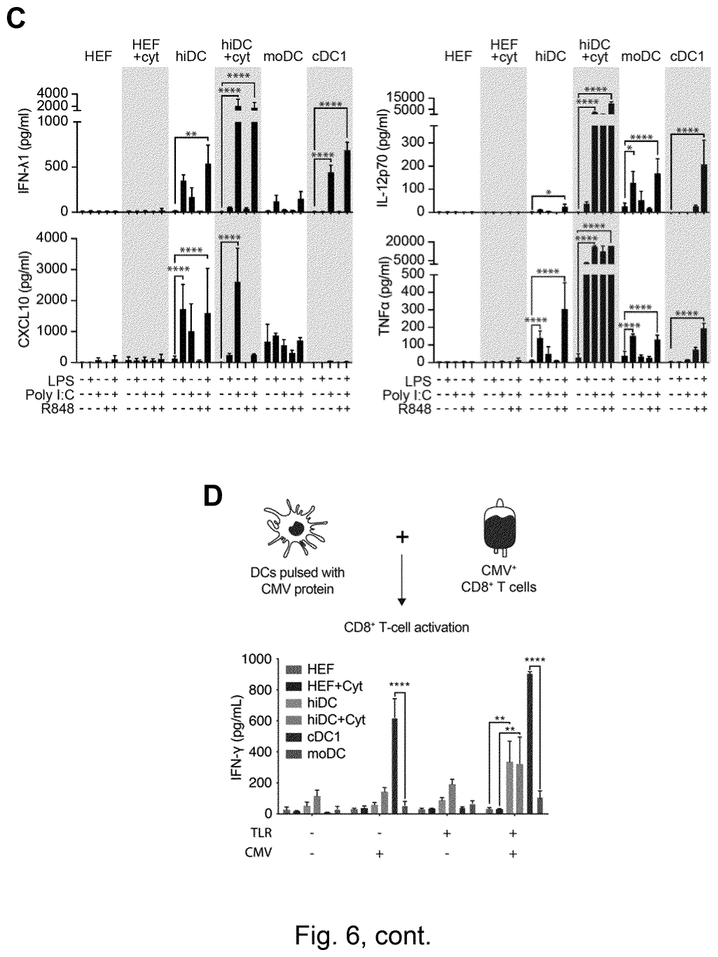

The inventors observed that both hiDCs and cDC1 upregulated co-stimulatory molecules after TLR3 or combined stimuli (FIG. 6A). In addition, hiDCs responded to higher extent to TLR4 triggering than cDC1s. Accordingly, cDC1s differentiated from CD34⁺ hematopoietic progenitors respond to LPS, pointing out to a general feature of in vitro generated cDC1-like cells (Balan et al. 2018). To evaluate phagocytic capacity, short incubations with labeled dead cells were performed. The inventors observed hiDCs (47.5±12.0%), hiDCs generated in the presence of IFN-γ, IFN-β and TNF-α (19.4±7.7%) and cDC1s (10.9±3.5%) incorporated dead cell material (FIG. 6B-D), a critical feature of cross-presenting DCs. DC maturation and phagocytosis are often inversely correlated (Broz et al. 2014). Accordingly, hiDCs generated in the presence of cytokines expressed higher levels of co-stimulatory molecules and showed decreased capacity to incorporate dead cells (FIG. 6A-C). To confirm that hiDCs also provide the third signal required for T-cell activation, cytokine secretion was evaluated (FIG. 6F). It was first observed that hiDCs and cDC1s responded to TLR3 challenge by secreting the human cDC1-specific cytokine IFN-λ1 (Hubert et al. 2020). This comes in contrast to moDCs that were unresponsive to TLR3 agonists (Lauterbach et al. 2010). In addition, hiDCs also responded to TLR4 and 3 by secreting IL12p70, CXCL10 and TNF-α. Moreover, it was observed that IFN-γ, IFN-β and TNF-α increased the magnitude of cytokine secretion. The inventors then asked whether hiDCs cross-present antigens to CD8⁺ T-cells. HEFs, moDCs, cDC1s and hiDCs pulsed with CMV protein were co-cultured with CD8⁺ T cells isolated from CMV⁺ donors. As readout for T-cell activation, IFN-γ secretion was quantified (FIG. 6G). As expected, it was observed that cDC1s, in contrast to moDCs or HEFs, efficiently cross-presented CMV antigens to CD8⁺ T cells. Strikingly, the inventors observed that hiDCs generated with or without cytokines established the ability to cross-present antigens to CD8⁺ T-cells. Together, these data support that reprogrammed hiDC are functional cross-presenting DCs.

### CONCLUSION

Together, these data support hiDC affiliation to the cDC1 subset as well as their acquired ability to respond to inflammatory stimuli, engulf dead cells, secrete cytokines and cross-present antigens enabling the activation of antigen-specific CD8⁺ T cells.

### Example 10. Efficient Reprogramming of Human Adult Somatic Cells

The generation of cDC1s from human accessible cell types could represent an additional source of DCs for cancer immunotherapy. Therefore, the inventors attempted to reprogram primary human dermal fibroblasts (HDFs) and mesenchymal stromal cells (MSCs) to cDC1-like cells with the improved DC reprogramming protocol.

### Methods

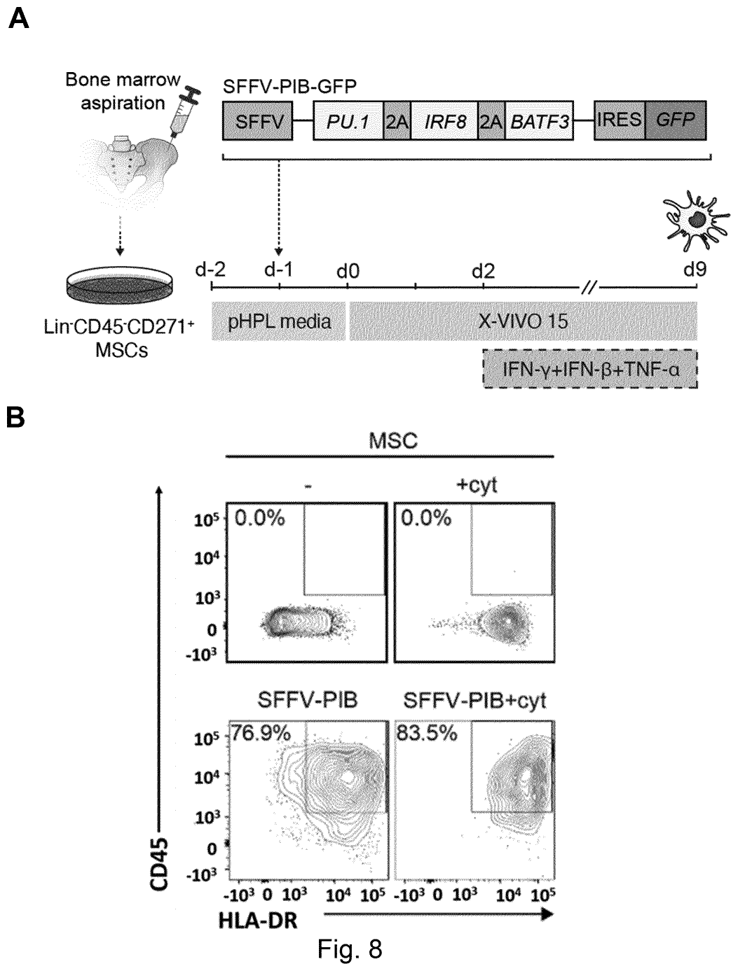

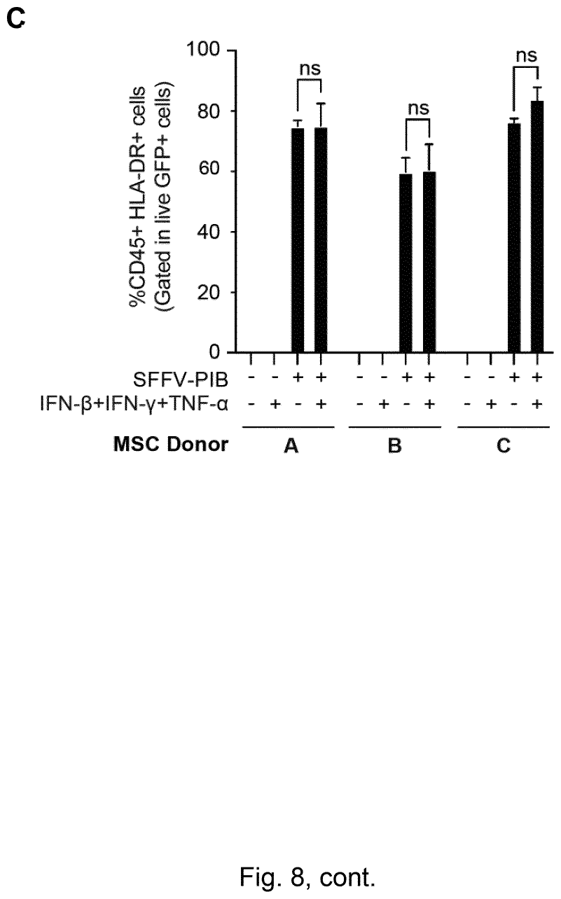

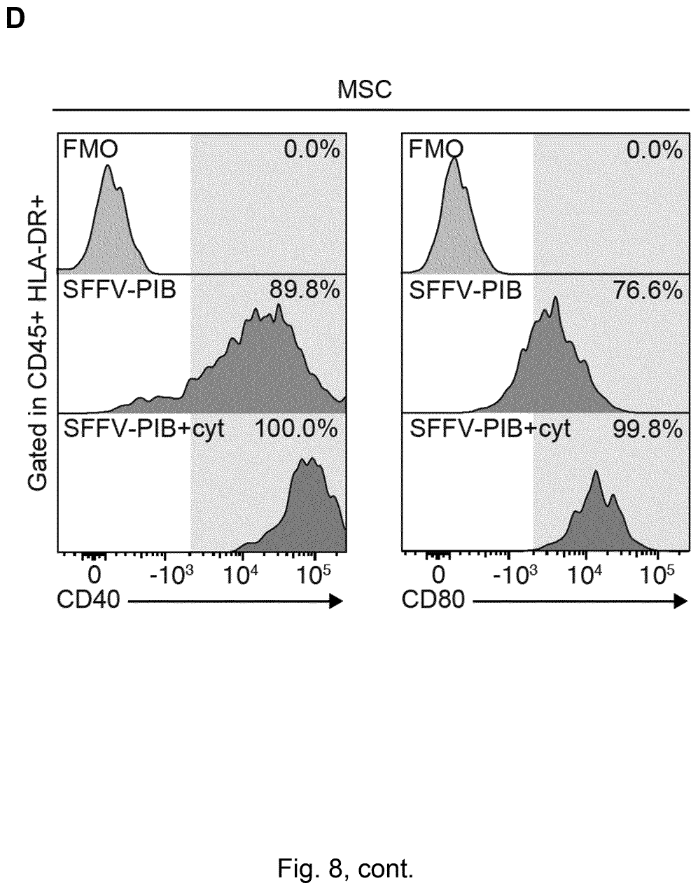

HDFs from 3 healthy donors were obtained and evaluated for cDC1 reprogramming efficiency. Single cell transcriptomes were generated for HDF-derived hiDCs and scPred analysis was used for DC subset affiliation. Purified MSCs from 3 healthy donors were transduced with SFFV-PIB lentiviral particles and cultured in chemically defined, serum-free X-VIVO 15 media (FIG. 8A). The cells were evaluated for cDC1 reprogramming efficiency. For experimental details, see previous Examples.

### Results

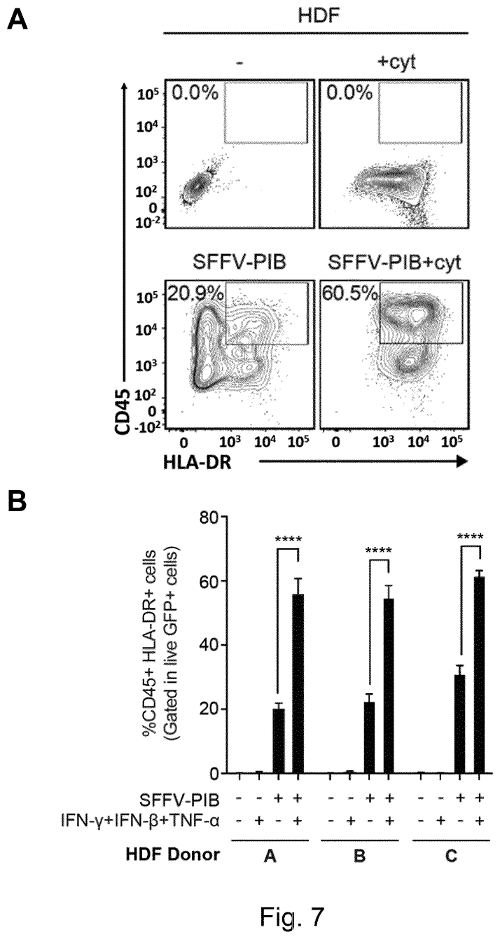

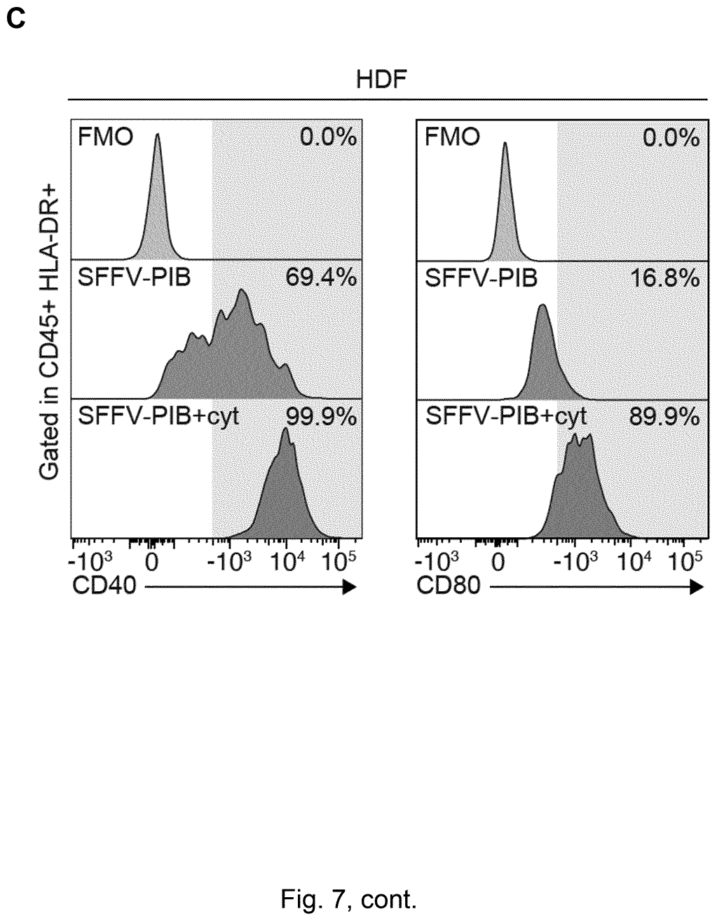

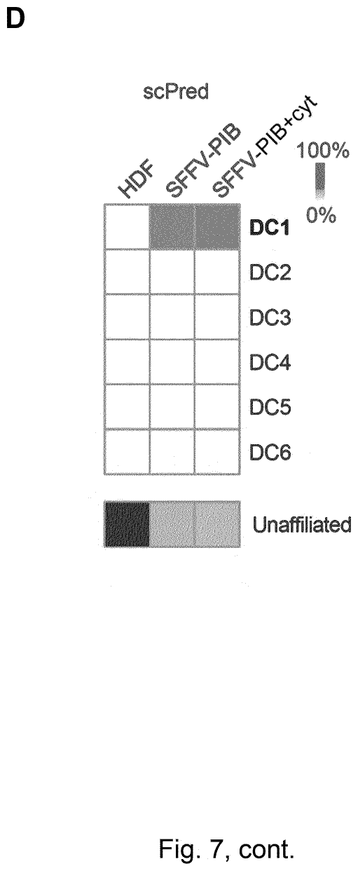

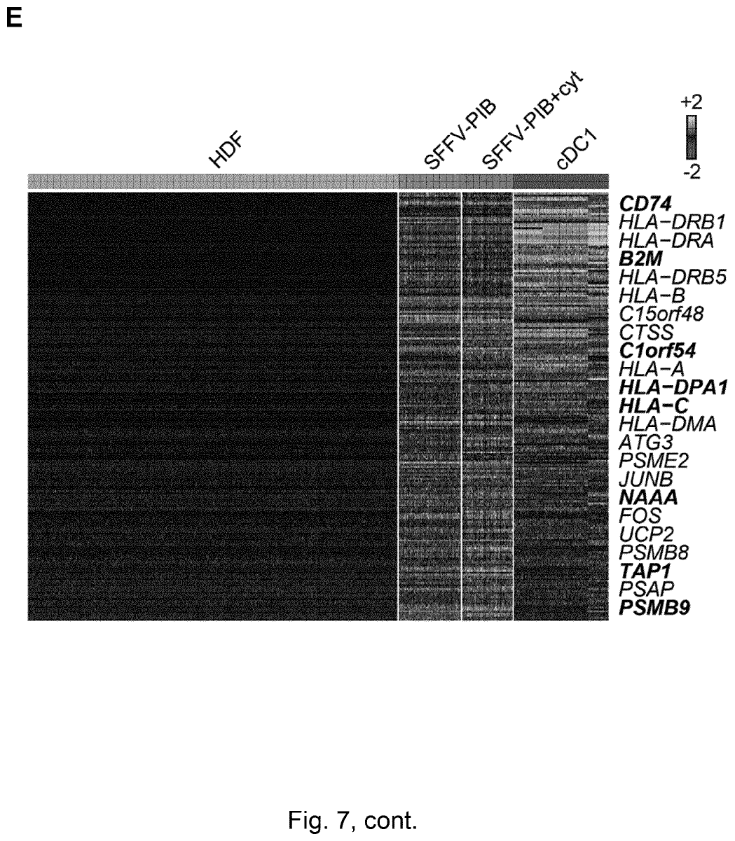

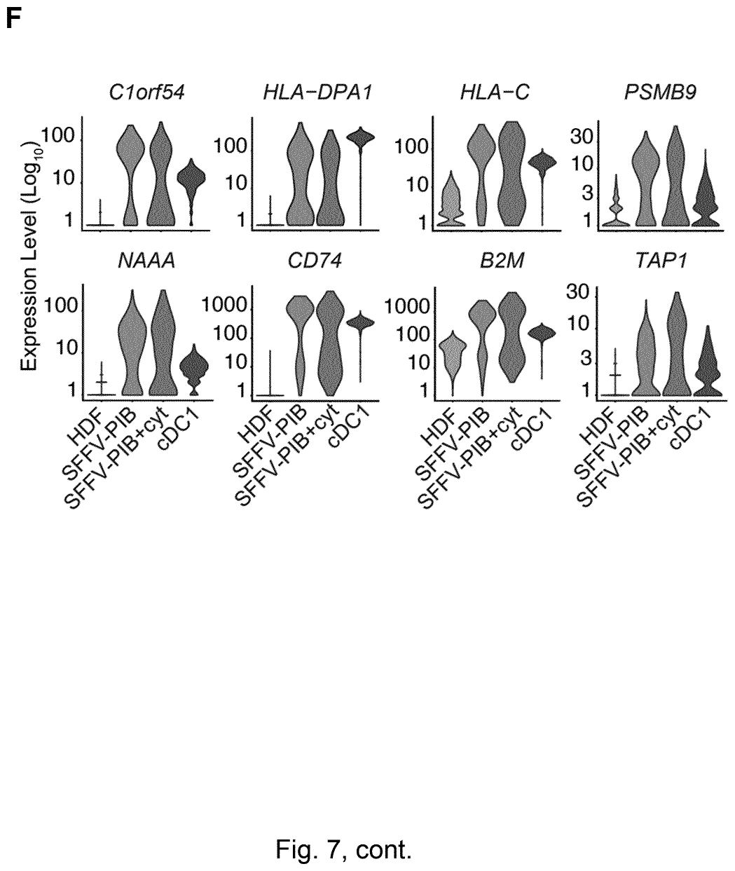

The efficiency of hiDC generation ranged from 20-35% across donors using SFFV-PIB (FIG. 7A-B). When combined with IFN-γ, IFN-β and TNF-α reprogramming efficiency increased ˜2-fold (FIG. 7A-B) and also lead to an increase in CD40 and CD80 (FIG. 7C). scPred analysis assigned 60.6% and 59.3% of HDF-derived hiDCs generated with and without cytokines, respectively, to the cDC1 subset (FIG. 7D). cDC1 identity was further confirmed by the expression of the cDC1-specific genes C1orf54 and HLA-DPA1 and antigen processing and presentation genes CD74, HLA-C, B2M, PSMB9, NAAA and TAP1 (FIG. 7E-F). 60-75% of MSCs from the 3 donors converted into hiDCs (CD45⁺ HLA-DR⁺) co-expressing CD40 and CD80 (FIG. 8B-D). IFN-γ, IFN-β and TNF-α did not further improve the generation of hiDC1s from MSC cultures.

### CONCLUSION

These data suggest that PU1, IRF8 and BATF3 induce a cDC1 fate in human adult cells and MSCs, highlighting the consistency of the reprogramming method across multiple cell types and donors. The lack of effect adding cytokines in MSCs, suggests that inflammatory cytokine signalling may facilitate cDC1 reprogramming in a cell-type specific fashion.

### Example 11. Induction of Anti-Tumour Immunity In Vivo

Considering the interest to evaluate whether iDCs generated by direct cell reprogramming are functional in vivo, the inventors took advantage of the mouse system and asked whether mouse iDCs (derived from Clec9a-tdTomato reporter MEFs, tdTomato⁺ cells) induce anti-tumour immunity using syngeneic cancer mouse models.

### Methods

To access antigen cross-presentation, CD8⁺ T-cells from spleen of OT-I mice were enriched using a naïve mouse CD8⁺ T-cell Isolation kit (Miltenyi). Enriched CD8⁺ T-cells were labeled with 5 μM Cell Trace Violet CTV (Thermo Fisher) at room temperature for 20 min, washed, and counted. FACS-sorted tdTomato+(generated with SFFV-PIB) at indicated time points and cDC1-like BM-DCs were incubated at 37° C. with OVA protein (10 μg/ml) in the presence of Poly I:C (1 μg/ml) for 10 hours. After extensive washing, 20,000 DCs were incubated with 100,000 CTV-labeled OT-I CD8⁺ T-cells in 96-well round-bottom tissue culture plates with Poly I:C (1 μg/ml). After 3 days of co-culture, T-cells were collected, stained, and analyzed in BD LSR Fortessa. T-cell proliferation (dilution of CTV staining) was determined by gating live single TCR⁺ CD8⁺ T-cells. Levels of mouse IFN-α and Cxcl10 were assessed in 50 μl of culture supernatants of purified tdTomato⁺ cells at day 9 using the LEGENDplex Mouse Anti-Virus Response Panel (BioLegend). LPS (100 ng/ml) or Poly I:C (1 μg/ml) were added overnight. Acquisition was performed in a FACSCanto, and data were analyzed using LEGENDplex (BioLegend) software.

For the in vivo experiments, B16-OVA (0.5×10⁶) tumor cells were injected subcutaneously into the left flank of 6-10 week-old C57Bl/6 females. FACS-sorted tdTomato⁺ cells generated with SFFV-PIB at day 9 were mixed with B16-OVA cells before tumor implantation. Alternatively, tdTomato⁺ cells, MEFs transduced with SFFV-GFP control or CD103⁺ BM-DCs were injected intra-tumorally in established tumours at day 8 after tumor establishment. On the previous day, cells were stimulated with LPS (100 ng/ml) and Poly I:C (1 μg/ml) overnight. On injection day, cells were pulsed with OVA₂₅₇₋₂₆₄ peptide (5 μg/ml) at 37° C. for 30 minutes. After washing twice in PBS, 80,000 cells were resuspended in 60 μl PBS and injected intra-tumorally per each tumor-bearing mice at day 8 post-implantation of B16-OVA tumours. Tumor size was measured with a caliper [Volume=0.5×length×width×height] every one to two days over the indicated periods of time. For evaluation of T-cell infiltration and activation, after injection of iDCs in the tumor bearing animals, 1.5×10⁶ CTV-labeled OT-I CD8⁺ T-cells were injected intravenously. After 4 days, animals were sacrificed and tumours and tumor-draining lymph nodes were collected and digested mechanically and chemically with collagenase D (1 mg/ml) and DNAse I (10 mg/ml). Dead cells were eliminated using Percoll. For intracellular cytokine analyses, cells were re-stimulated for 4 h at 37° C. and 5% CO2, in complete RPMI medium in the presence of phorbol 12-myristate 13-acetate (100 ng/ml) and ionomycin (1 μg/ml). GolgiPlug solution (1 μl/ml) was added to the culture medium for the last 2.5 h. Cells were stained with Fixable viability dye FITC for 30 min at 4° C. Intracellular staining for IFN-γ and Granzyme B were performed using the Intracellular Fixation & Permeabilization buffer set. Data were acquired using Gallios and BD LSRFortessa.

### Results

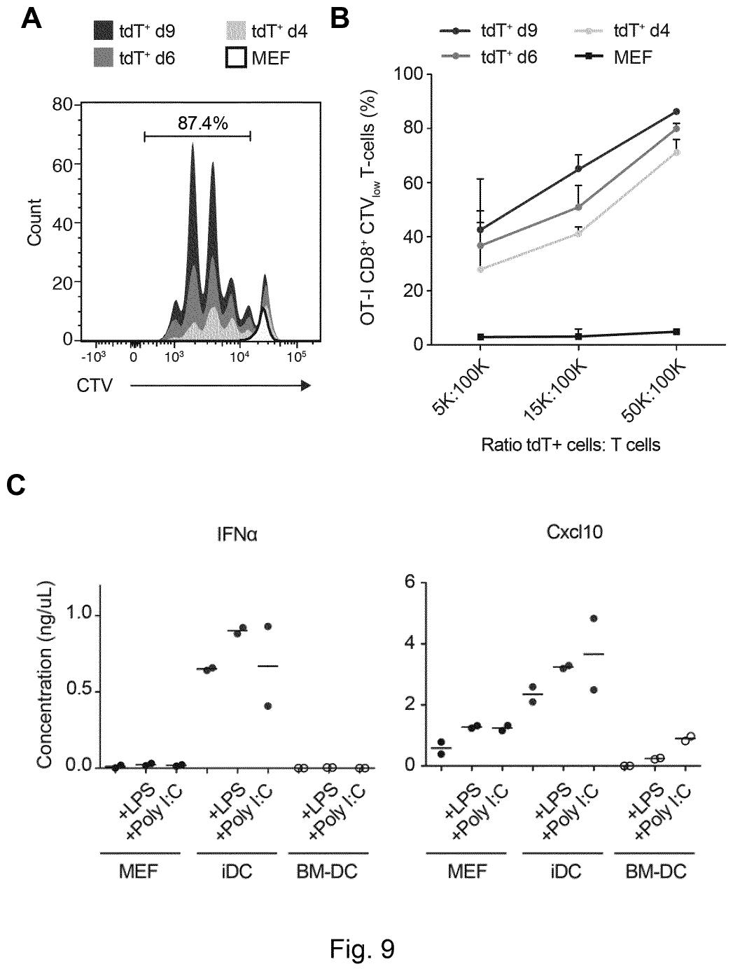

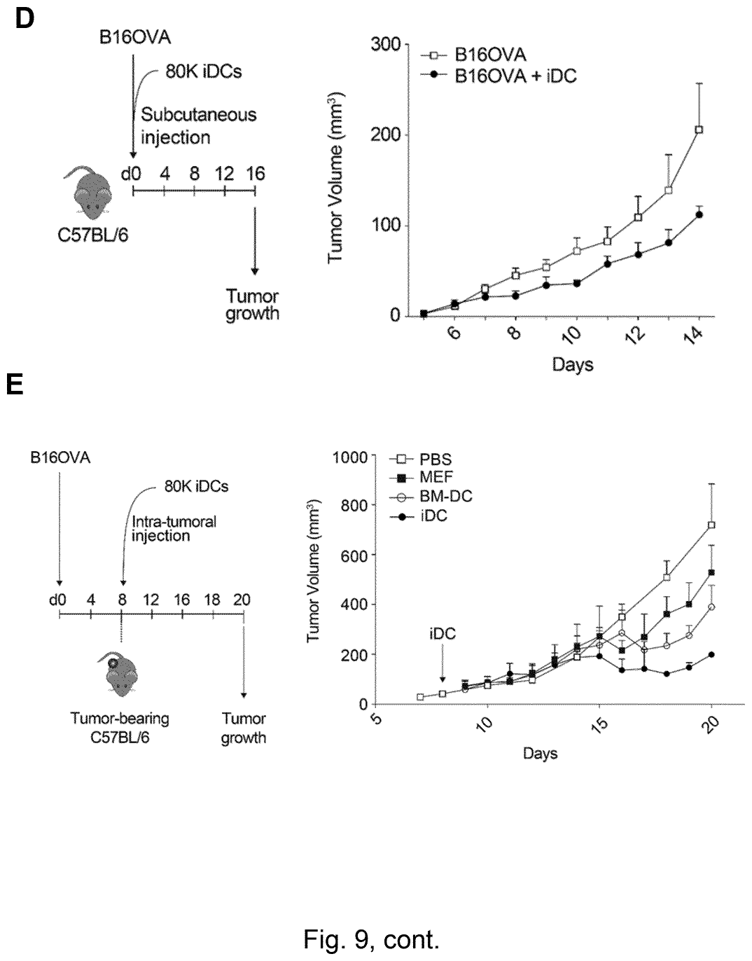

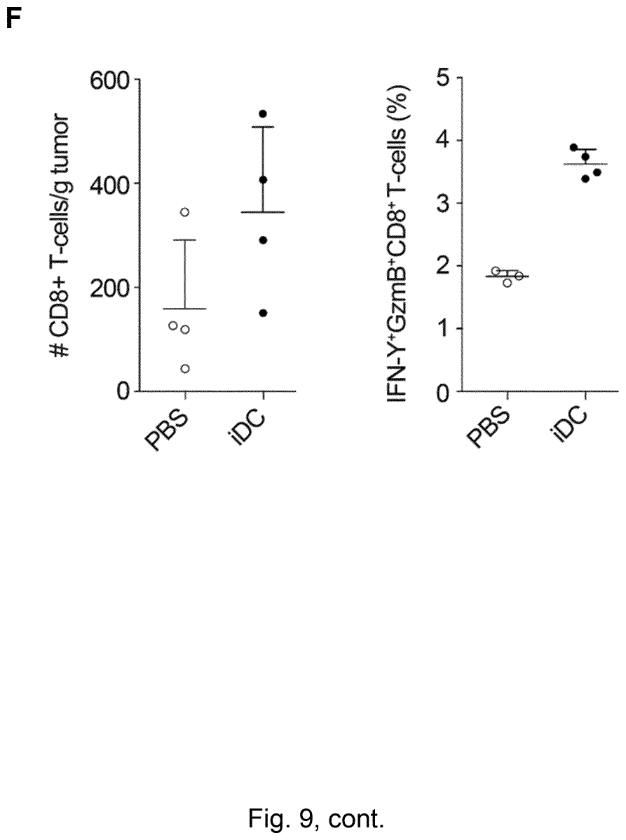

The iDCs were able to perform cross-presentation antigens already at day 4 and 6 of reprogramming (FIG. 9A-B). Furthermore, purified tdTomato⁺ iDCs secreted Cxcl10 and IFNα previously described as essential for cDC1-mediated tumor rejection (FIG. 9C) (Diamond et al., 2011). It was further observed that co-injection with iDCs reduced tumor growth during tumor establishment (FIG. 9D). Remarkably, a single intra-tumoral injection of 80,000 iDCs in established tumours was sufficient to slow down tumor growth (FIG. 9E). Intra-tumoral injection of non-reprogrammed MEFs and CD103⁺ BM-DCs was not as efficient in controlling tumor growth. In addition, injection of iDCs increased the infiltration of antigen-specific CD8⁺ T-cells in the tumor as well as promoted an increased cytotoxic profile of T-cells in the tumor-draining lymph nodes in both models (FIG. 9F).

### CONCLUSION

These data support the hypothesis that iDCs induce anti-tumor immune responses. Together, these data suggest that iDCs control tumor growth by activating antigen-specific CD8⁺ T-cells and promoting their infiltration within tumours.

### Example 12. Efficient DC Reprogramming Requires Combined Action of PU.1, IRF8 and BATF3

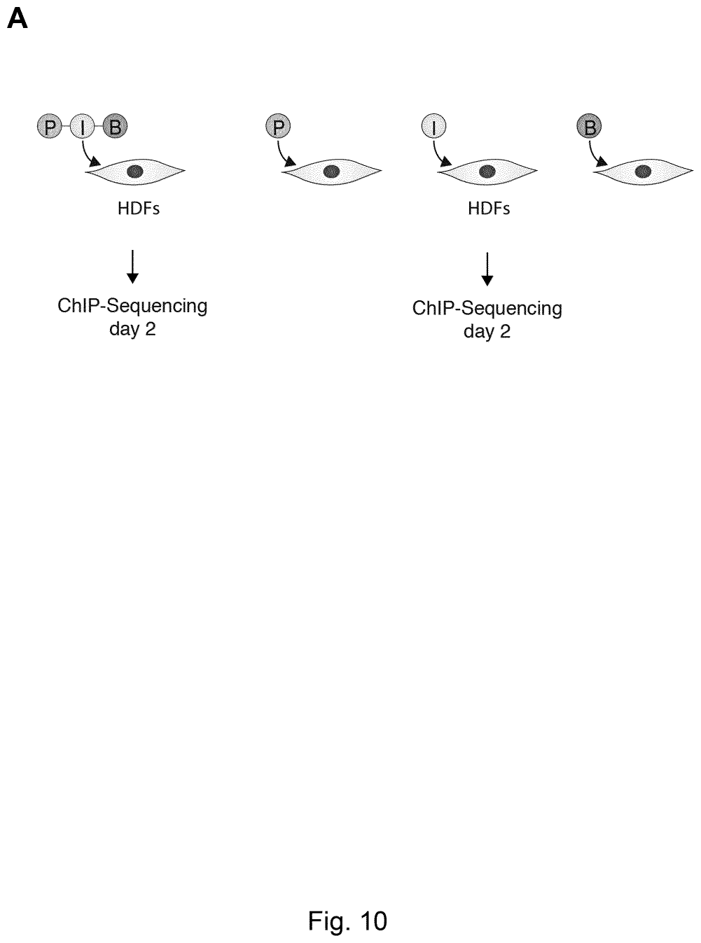

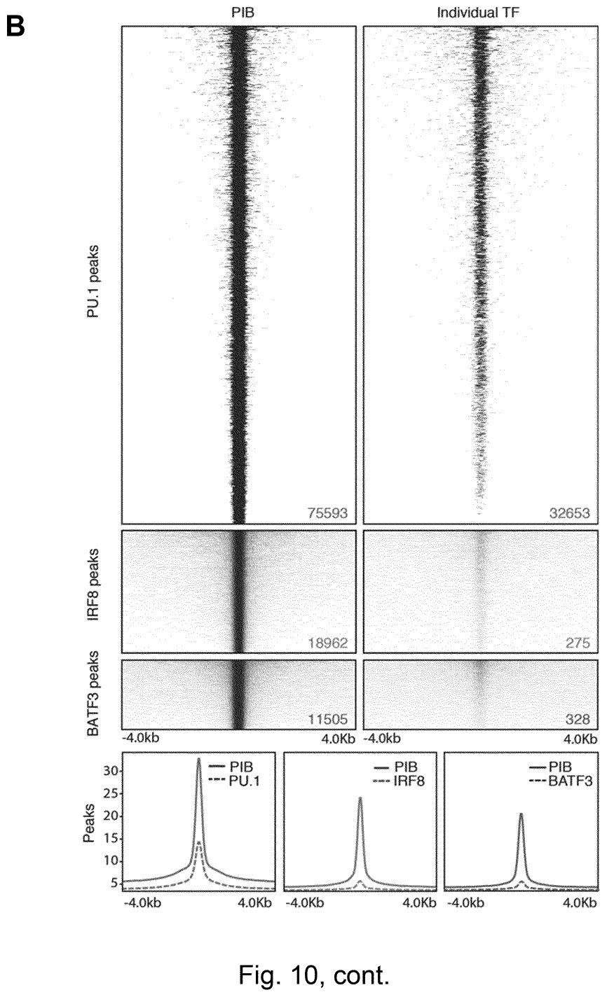

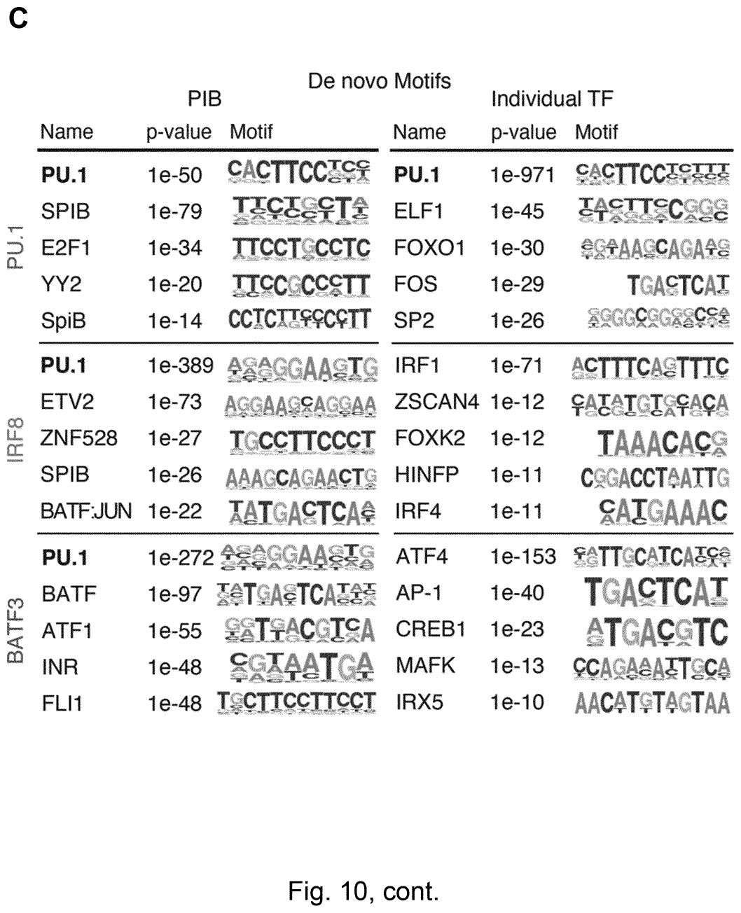

To shine light on the molecular mechanism underlying DC reprogramming mediated by PU.1, IRF8 and BATF3, the inventors transduced HDFs with Dox-inducible lentiviral particles encoding the three reprogramming factors simultaneously or each one individually, and performed ChIP-seq for PU.1, IF8 and BATF3 48 h after TF induction (FIG. 10A)

### Methods

### ChIP-Sequencing

TFs were delivered with a polycistronic lentiviral vector (pFUW-tetO-PIB) or individual vectors (pFUW-tetO-PU.1, pFUW-tetO-IRF8, or pFUW-tetO-BATF3) with pFUW-M2rtTA. ChIP was performed 48 hours after the addition of Dox.

Chromatin in cultured cells was fixed by adding 1/10 volume of freshly-prepared formaldehyde solution [11% Formaldehyde (Sigma), 0.1M NaCl, 1 mM EDTA and 50 mM HEPES] to each cell suspension in complete DMEM. Tubes were left 15 minutes at room temperature with agitation. Fixation was stopped by adding 1/20 volume of 2 mM Glycine solution (Sigma). After 5 minutes incubation, cells were centrifuged at 800 g for 10 min at 4° C. Cell pellets were resuspended in 10 ml chilled PBS-Igepal, 100 μl PMSF were added to each tube and centrifuged at 800 g for 10 minutes at 4° C. Cell pellets were snap-frozen on dry ice and stored at −80° C. Active Motif (Carlsbad, CA) prepared chromatin, performed ChIP, generated libraries and sequenced libraries. Briefly, chromatin was isolated by adding lysis buffer, followed by disruption with a Dounce homogenizer. Lysates were sonicated and the DNA sheared to an average length of 300-500 bp (Active Motif's EpiShear probe sonicator). Genomic DNA (Input) was prepared by treating aliquots of chromatin with RNase, proteinase K and heat for de-crosslinking, followed by clean up using solid phase reversible immobilization (SPRI) beads (Beckman Coulter) and quantitation by Clariostar (BMG Labtech). Extrapolation to the original chromatin volume allowed determination of total chromatin yield. 30 μg of chromatin was pre-cleared with protein A/G agarose beads (Invitrogen). Immunoprecipitations were performed with 4 μg of antibodies against human PU.1, IRF8 and BATF3 (rabbit anti-human PU.1, rabbit anti-human IRF8 or sheep anti-human BATF3). Complexes were washed, eluted from the beads with SDS buffer, and subjected to RNase and proteinase K treatment. Crosslinks were reversed by incubation overnight at 65° C., and ChIP DNA was purified by phenol-chloroform extraction and ethanol precipitation. To confirm ChIP enrichment, quantitative PCR (QPCR) reactions were carried out in triplicate on specific genomic regions using SYBR Green Supermix (Bio-Rad). Resulting signals were normalized for primer efficiency by carrying out QPCR for each primer pair using Input DNA. Illumina sequencing libraries were prepared from ChIP and Input DNAs by standard consecutive enzymatic steps of end-polishing, dA-addition, and adaptor ligation. Steps were performed on an automated system (Apollo 342, Wafergen Biosystems/Takara). After a final PCR amplification step, the resulting DNA libraries were quantified and sequenced on Illumina's NextSeq 500 (75 nt reads, single end).

### ChIP-Sequencing Analysis and Data Visualization

ChIP-seq analysis was performed on the raw FASTQ files. FASTQ files were mapped to the human hg38 genome using Bowtie 2 program allowing for 2 base pair mismatches. Mapped output files were processed through MACS v2.1.0 analysis software to determine peaks. Peak annotation was performed using ChIPseeker R library. For genome tracks, bigwig files were created from bam files with deeptools (https://deeptools.readthedocs.io/en/develop/) and explored using the UCSC Genome Browser. For Chromatin State Fold-Enrichment analysis, enrichment scores for genomic features, such as PU.1, IRF8 and BATF3 ChIP-seq peaks and histone marks were calculated using ChromHMM Overlap Enrichment (http://compbio.mit.edu/ChromHMM/). ChromHMM segmentation, containing 18 different chromatin states, was downloaded from Roadmap website (http://www.roadmapepigenomics.org/tools) and used for analysis. Enrichment scores were calculated as the ratio between the observed and the expected overlap for each feature and chromatin state based on their sizes and the size of the human genome. For de novo motif discovery, findMotifsGenome.pl procedure from HOMER was used on PU1, IRF8 and BATF3 separately. HOMER was run using default parameters and input sequences comprising+/−100 bp from the center of the top 2,500 peaks. Co-bound regions by PU.1, IRF8 and BATF3 were found using findOverlapsOfPeaks function in ChIPpeakAnno R library (http://www.biomedcentral.com/1471-2105/11/237). Co-bound regions were used for de novo motif discovery using HOMER. In order to evaluate similarity of the two sets based on the intersections the inventors calculated Jaccard statistic using MACRO-APE (https://opera.autosome.ru/macroape/compare). To produce the heatmaps and profile plots, deeptools in Reference-point mode were used where each feature (such as peaks of a TF or histone marks) was aligned at PU.1, IRF8 or BATF3 summits and tiled the flanking up- and downstream regions within ±4 kb.

### Co-Immunoprecipitation (Co-IP)

Total cell extracts were prepared from HEK293T cells transfected with SFFV-PIB in three cell densities (1, 2, 5 million cells) in IP lysis buffer (Thermo Fisher) supplemented with protease inhibitors [1× Halt Protease Inhibitor Cocktail (Thermo Fisher), 1 mM PMSF, 5 mM NaF]. ChIP-grade Protein A/G Magnetic beads were incubated with 5 μg of each antibody (rabbit anti-human PU.1, rabbit anti-human IRF8 or sheep anti-human BATF3) for 2 hours. Cell lysates were pre-cleared with non-antibody-treated ChIP-grade protein A/G beads for 1 hour and then incubated with antibody-treated beads for 1 hour. The supernatant was removed and beads were washed three times using Tris-buffered saline with 0.1% Tween 20 detergent (TBST). Input controls were performed using 10% of non-immunoprecipitated samples (2 million cell density). As a control, lysates (2 million cell density) were immunoprecipitated with 5 μg of a rabbit IgG isotype (Invitrogen). Samples were eluted by boiling in Laemmli sample buffer and processed for western blotting. For immunoblotting, membranes were blocked with TBST buffer containing 3% milk, incubated overnight with primary antibodies washed five times using PBS with 0.1% Tween 20 detergent (PBST), blocked with TBST buffer containing 3% milk for 45 min, incubated with HRP-conjugated secondary antibodies for 1 hour, washed four times with PBST and then detected by ECL (Thermo Scientific) in a Chemidoc (Bio-Rad).

### Results

PU.1-Dominant Chromatin Targeting Capacity in cDC1 Reprogramming

To shine light on molecular mechanisms underlying DC reprogramming mediated by PU.1, IRF8 and BATF3, the inventors expressed the three reprogramming factors in combination or individually in HDFs, and performed chromatin immunoprecipitation sequencing (ChIP-seq) at early stages of reprogramming (48 h, FIG. 10A). First, PU.1 showed the highest chromatin binding (75,593 peaks), followed by IRF8 (18,962 peaks) and BATF3 (11,505 peaks) when factors were co-expressed (FIG. 10B). Interestingly, it was observed that >40% of PU.1-bound peaks were similar between individual and combined expression, suggesting that PU.1 has independent targeting capacity that is augmented when IRF8 and BATF3 are available. In sharp contrast, IRF8 and BATF3 peaks were scarce when these transcription factors were expressed individually (<3% of peaks when compared to combined expression), suggesting that IRF8 and BATF3 require cooperative binding with PU.1 to engage chromatin and induce cDC1 fate. De novo motif prediction analysis for PU.1 peaks showed strong enrichment for PU.1 motif when expressed individually or in combination (FIG. 10C). While IRF8 and BATF3 expressed individually showed enrichment in IRF and AP-1 motifs respectively, the PU.1 motif was highly enriched for these transcription factors when expressed in combination. These data go in line with recent findings describing PU.1 as a non-classical pioneer transcription factor capable of redistributing partner transcription factors in human myeloid and lymphoid cells (Minderjahn et al. 2020), and highlight transcription factor cooperative dynamics at early stages of cDC1 reprogramming.

### Cooperative Binding of PU.1, IRF8 and BATF3 at Promoters and Enhancers at Open Chromatin

Next, the inventors investigated the overlap between PU.1, IRF8 and BATF3 chromatin targets. 5,383 genomic positions were shared between the three reprogramming factors when expressed together, representing 28% and 47% of total IRF8 and BATF3 peaks, respectively (FIG. 11A). De novo motif prediction for the PIB overlapping peaks showed enrichment for PU.1-IRF and BATF motifs (FIG. 11B), which also displayed some overlap and similarity (Jaccard similarity index=0.02) (FIG. 11C). These data suggest that PU.1, IRF8 and BATF3 interact physically. To test this hypothesis, co-immunoprecipitation (co-IP) was performed and interactions between the 3 factors was confirmed (FIG. 11D). Next, the differentially expressed genes between HDFs and hiDC d9 that were bound by at least one of the reprogramming factors were plotted and it was observed that they contain both downregulated fibroblast genes and upregulated cDC1-associated genes, including SLAMF8 and TACSTD2 (FIG. 11E). To dissect whether PU1, IRF8 and BATF3 binding occurs at open or closed chromatin regions, the inventors took advantage of publicly available ChIP-seq datasets for histone marks in HDFs and used ChromHMM chromatin segmentation (Ernst and Kellis 2012) for visualization. It was observed that PIB co-bound peaks were enriched mainly at promoter and enhancer regions (FIG. 11F). A small fraction (12%) of peaks associated with bivalent chromatin marked either by H3K4me1, H3K4me3 and H3K27me3 or H3K4me1 and H3K27me3 was also observed.

### CONCLUSION

Together, our data support a model where PU.1 binds mainly to active promoters and enhancers localized open chromatin sites and recruits IRF8 and BATF3 to silence the original fibroblast genes and gradually impose the cDC1 transcriptional program (FIG. 11G).

### Example 13. Efficient DC Reprogramming of Mouse and Human Cancer Cells

Given that ectopic expression of PU1, IRF8 and BATF3 induces cDC1-like fate in mouse and human fibroblasts, the inventors hypothesized that the enforced expression of the same combination of TFs in cancer cells would convert them into antigen-presenting cDC1 and surpass one of the main problems in tumour immunity—the loss of antigen-presenting machinery.

### Methods

### Cancer Cell Reprogramming

Cancer cell lines were seeded at a density of 60 000 cells/mL in 6-well plates and incubated overnight with SFFV-PIB-GFP lentiviral supernatants, supplemented with polybrene (8 μg/mL). Media was changed every 2 days for the duration of the cultures. Whenever cells reached 80-90% confluency, cells were seeded at 1:6 dilution on 10 cm plates. Flow cytometry was used to access DC reprogramming efficiency in mouse and human cancer cells.

### T Cell Priming and Antigen Cross-Presentation Assays

CD8⁺ T cells from spleen of OT-I mice were enriched using a naïve mouse CD8⁺ T cell Isolation kit (Miltenyi). Enriched CD8⁺ T cells were labelled with CTV according to manufacturer's protocol. MACS-sorted reprogrammed cells, non-reprogrammed cancer cells, eGFP transduced cancer cells and CD103⁺ BM-DCs were incubated at 37° C. with OVA peptide (SIINFEKL, T cell priming assays) or protein (cross-presentation assays). OVA expressing cells were not incubated with exogenous OVA. Cells were incubated overnight in the presence of Poly(I:C) or IFN-γ where indicated. 5×10³ antigen presenting cells were incubated with 1×10⁵ CTV-labelled OT-I CD8⁺ T cells in 96-well round-bottom untreated-tissue culture plates. After 3 days of co-culture, T cells were collected, stained for viability (fixable viability dye eFluor-520, eBioscience), CD8α, TCR-β, and CD44 and analysed by flow cytometry. T cell proliferation (dilution of CTV) and activation (CD44 expression) were determined by gating on live, single, TCR-β⁺ and CD8⁺ T cells. Threshold for data plotting was fixed at 1,000 events within live cell gating.

### T Cell Killing Assays

CD8⁺ T cells from spleen of OT-I mice were enriched using a mouse CD8⁺ T cell isolation kit (Miltenyi) according to manufacturer's protocol. 6-well untreated plates were coated with anti-CD3 and anti-CD28 at 2×10⁻³ mg mL⁻¹ for 2 h at 37° C. and washed 3× before 1×10⁶ T cells per mL were seeded in complete growth media (RPMI) supplemented with murine IL-2 (Peprotech, 100 U mL-1) and murine IL-12p70 (Peprotech, 2.5×10−3 mg mL-1). After 24 h of activation, T cells were re-seeded at 1×10⁶ cells per mL in fresh complete RPMI supplemented with murine IL-2 for 48 h on new untreated plates to allow T cell expansion. MACS-sorted reprogrammed mOrange⁺ B16-OVA cells or IFN-γ treated cells were seeded with non-fluorescent B16-OVA (mOrange−) in equal numbers, 24 h before co-culture with T cells. Expanded T cells were added in ratios of 0:1, 1:1, 5:1, 10:1 T cell to target cell. B16 cells that do not express OVA were used to assess assay specificity. For flow cytometry analysis, cells were resuspended and stained for viability (DAPI) and anti-CD3 and measured at indicated time points post co-culture with T cells.

### Tumour Induction and Injection

B16-OVA tumours were established by subcutaneous injection of 2-5×105 tumour cells into the right flank of 6-10-week-old C57BL/6 females. Reprogrammed tumour-APCs were generated by transduction of B16 with SFFV-PIB. On day 5 after transduction and at day 7, 10 and 13 post-tumour establishment, tumour-APCs were purified by MACS with anti-MHC-II antibodies and 2×10⁵-3×10⁵ cells, resuspended in 100 μL of PBS and injected intra-tumorally. PBS or cells transduced with control lentiviruses were injected into tumours as controls. 24 h before injection, tumour cells or tumour-APCs were stimulated with Poly(I:C) and loaded with OVA. Mice were followed for survival and tumour size was measured with a calliper [Volume=π/6×L×W×H] every 2 days post-tumour establishment until the endpoint. Mice were sacrificed when the tumour surpassed 1500 mm3 in volume.

### Results

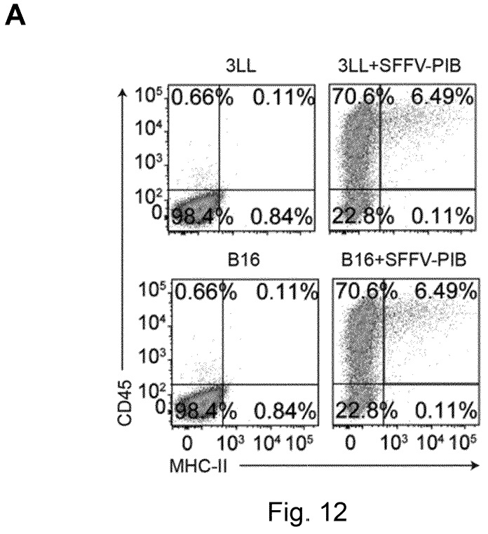

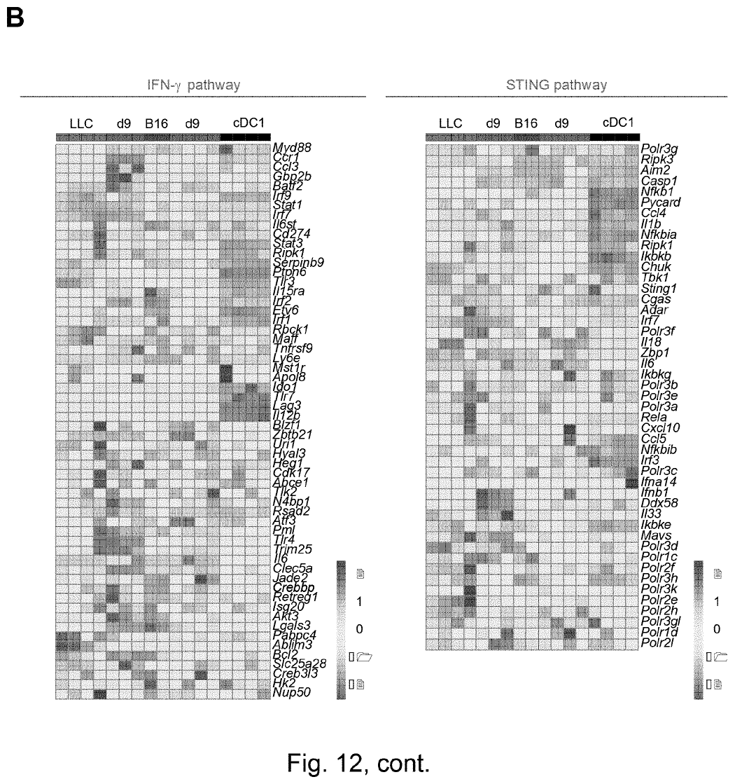

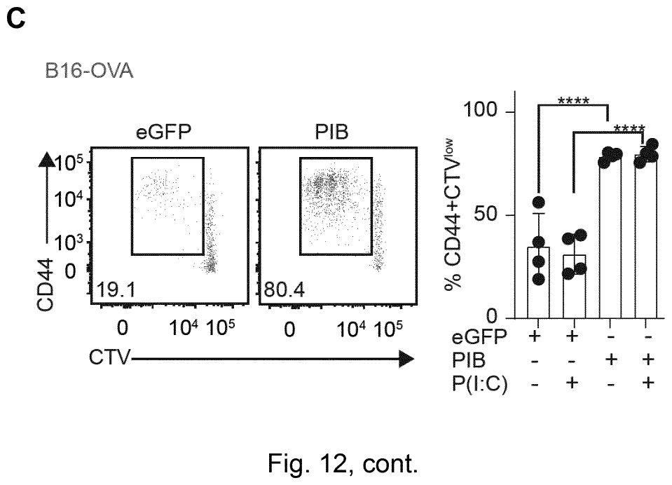

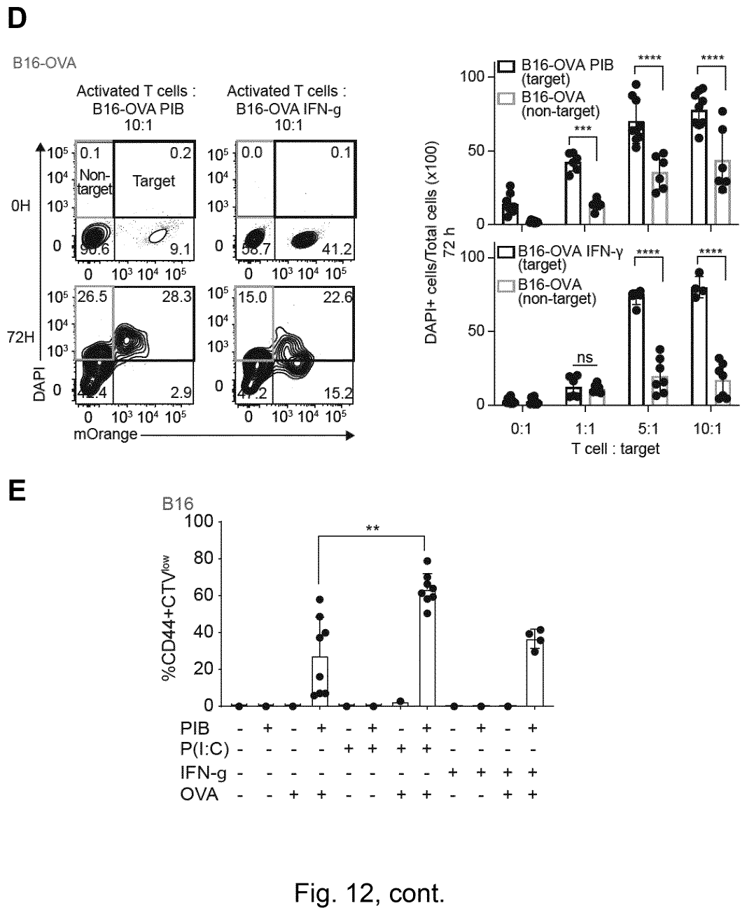

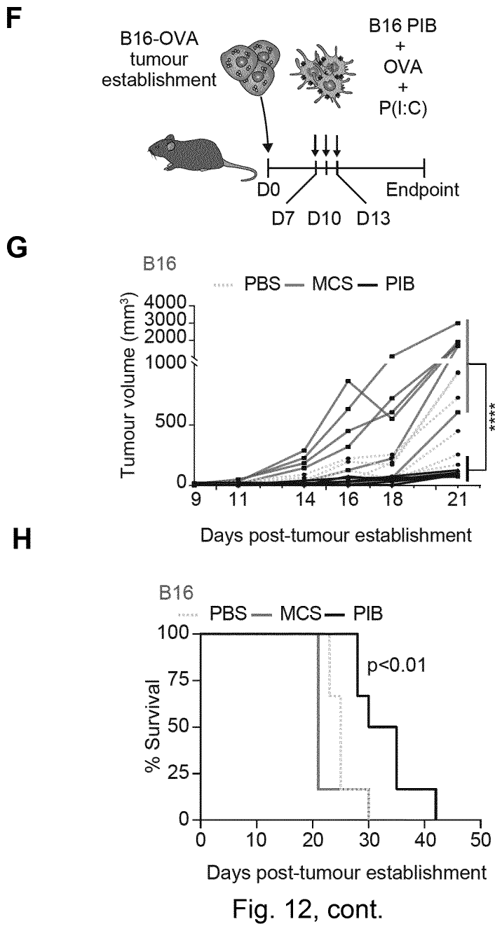

First, SFFV-PIB-IRES-GFP lentiviral supernatants were used to overexpress PIB in 3LL and B16, murine lung adenocarcinoma and melanoma cells, respectively. Both murine cancer cell lines are derived from C57BL/6 background and widely used in syngeneic mouse models for tumour immunity. The emergence of a double positive population for MHC-II and CD45 9 days after transduction was observed (FIG. 12A). Recent CRISPR screening approaches have highlighted the importance of IFN-γ signalling in unlocking anti-tumour immunity and cytotoxic T lymphocyte (CTL) sensitivity. Interestingly, IFN and STING pathway gene signatures are upregulated in both B16, and LLC cells transduced with PIB (FIG. 12B), suggesting an acquired immunogenic profile during reprogramming. To further investigate whether tumour-APCs become immunogenic and present endogenously expressed antigens, the inventors took advantage of Ovalbumin (OVA) expressing B16 cell line (B16-OVA). Magnetic-activated cell sorting (MACS) enriched CD45⁺ MHC-II⁺ OVA-expressing tumour-APCs were co-cultured with naïve OT-I CD8⁺ T cells to evaluate priming. While control eGFP-transduced B16-OVA and LLC-OVA cells showed low OVA antigen presentation capacity after IFN-γ or P(I:C) stimulation, tumour-APCs were remarkably efficient in priming naïve OT-I CD8⁺ T cells independently of IFN-γ or P(I:C) treatment (FIG. 12C). Next, to assess whether tumour-APCs become prone to CTL killing, B16-OVA cells expressing the fluorescent protein mOrange. Tumour-APCs were generated or B16-OVA cells treated with IFN-γ (target, mOrange⁺) were mixed with untreated B16-OVA cells (non-target, mOrange−) and co-cultured for 3 days with increasing ratios of activated OT-I CD8⁺ T cells. First, it was observed that tumour-APCs were more susceptible to being killed by CD8⁺ T cells than untreated B16-OVA cells (FIG. 12D). In addition, tumour-APCs were more efficiently killed by T cells (42.42±6.2%) than IFN-γ-stimulated B16-OVA cells (12.31±7.1%), particularly at low (1:1) ratios. Interestingly, killing of the non-target population was also observed in tumour-APC co-cultures at higher T cell to target-cell ratios and later time-points (72 h). This bystander killing effect may reflect a sustained activation of T cells by reprogrammed cells, increasing non-target cancer cell clearance. Next, the inventors evaluated cross-presentation of tumour-APCs after a pulse with OVA protein. Strikingly, it was observed that tumour-APCs established competence to cross-present antigens to CD8⁺ T cells, which is further enhanced by TLR3 stimulation (63.5±8.5 vs 27.5±20.9%) (FIG. 12 E). The inventors then asked whether OVA-loaded tumour-APCs would elicit tumour growth control in vivo after intra-tumoral injection in established B16-OVA tumours (FIG. 12 F). Remarkably, injection of tumour-APCs resulted in reduced tumour growth and improved survival significantly when compared to mice injected with PBS or control virus (FIG. 12 G-H).

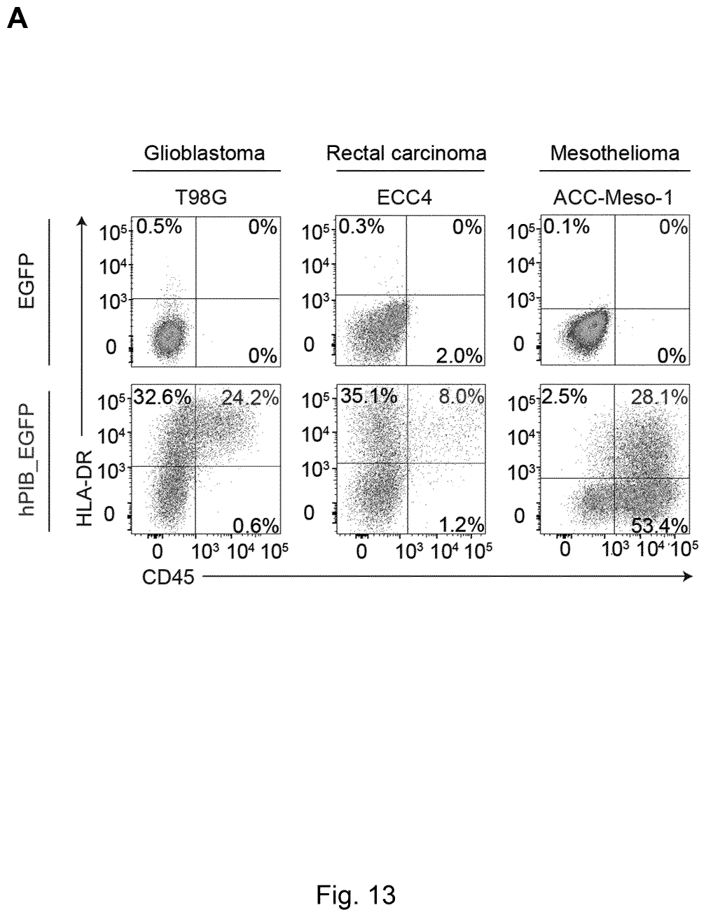

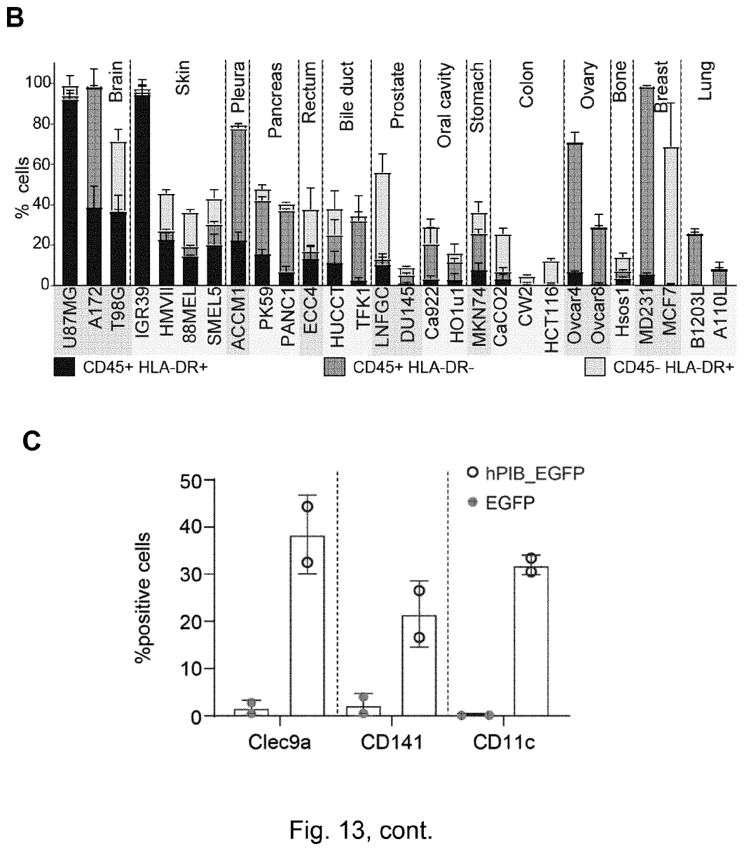

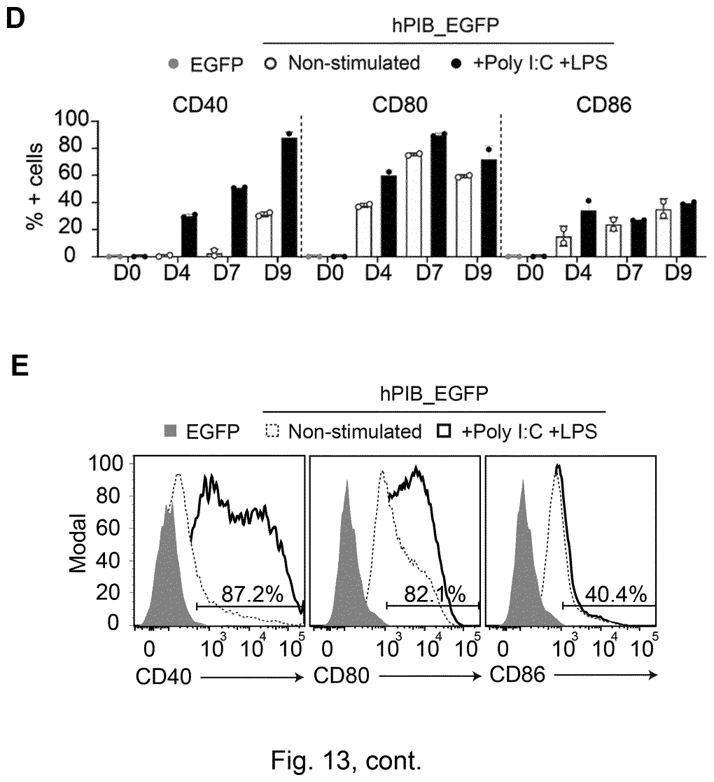

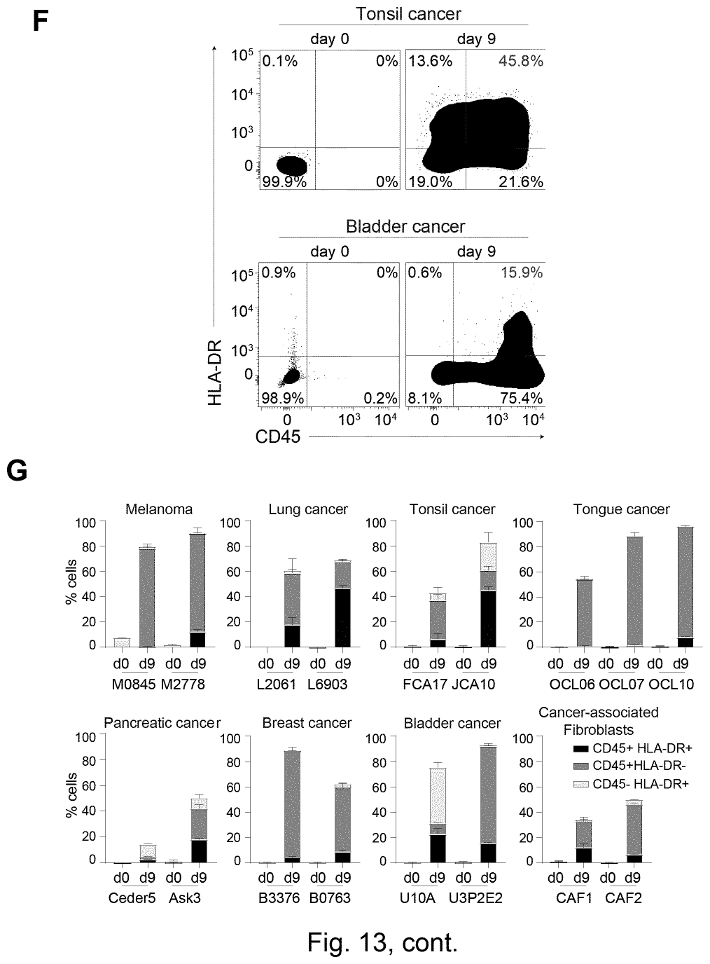

Then, to assess whether the cDC1 fate could be induced directly in human cancer cells, the expression of PU1, IRF8 and BATF3 was enforced in a panel of 28 human cancer cell lines. After 9 days, reprogramming efficiency as the percentage of transduced EGFP⁺ cells simultaneously expressing CD45 and HLA-DR was evaluated, reflecting hematopoietic commitment and antigen presentation capacity (FIG. 13A-B). The emergence of reprogrammed CD45⁺HLA-DR⁺ cells in all cell lines transduced with hPIB-IRES-EGFP was observed, but not in EGFP transduced controls, suggesting that cDC1 reprogramming is broadly applicable to human cancer cells. Furthermore, these data showed that cDC1 reprogramming efficiency ranged from 0.2±0.1% to 94.5±7.6% across cancer cell lines, independently of transduction levels and proliferation rates. Interestingly, despite low reprogramming efficiency in cancer cell lines from lung and breast carcinoma, large populations of cells acquiring either CD45 or HLA-DR expression were detected, which may represent partially reprogrammed cells that have acquired dendritic cell features (FIG. 13A-B). Human cancer cell-derived CD45⁺HLA-DR⁺ cells expressed cDC1 surface markers, including CLEC9A (59.1±3.6%), CD226 (67.5±1.8%), and CD11c (54.4±3.6%) (FIG. 13C). Given that productive activation of naïve T cells requires expression of co-stimulatory molecules, surface expression of CD40, CD80, and CD86 was evaluated. Human cancer cell-derived CD45⁺HLA-DR⁺ cells expressed these co-stimulatory molecules starting at day 4 increasing gradually until day 9. Importantly, tumour-APCs responded to TLR3/4 triggering (LPS and Poly I:C leading to an increase surface expression of co-stimulatory molecules, particularly in CD40 (88.2±3.8%. vs 31.3±1.8%) (FIG. 13D, E).

An important consideration for translation of tumour-APCs into therapy is whether reprogramming can be elicited in human primary cancer cells (FIG. 13F, G). To validate cDC1 reprogramming in primary cancer cells, 17 samples were collected from 7 different tumours obtained from patients with melanoma, lung, tonsil, tongue, pancreatic, breast and PDX-derived bladder carcinoma, as well as lung cancer associated fibroblasts (CAFs). Upon transduction with hPIB all primary cancer cells showed major phenotypic changes, initiating expression of CD45 and HLA-DR, reflecting reprogramming. Reprogramming efficiency ranged between 0.6%±0.4 to 47.0%±2.0. Samples from the same tumour types showed similar phenotypic profile indicating relatively low variability across patients. Interestingly, when compared to the panel of cell lines, primary cells were less resistant to reprogramming, illustrated by lung cancer cell lines (0.5%±0.1) and primary cells (47.0%±2.0). These data suggest that epigenetic barriers limiting reprogramming are less enforced in primary cells, opening avenues for broad applicability of cDC1 reprogramming to human tumours (FIG. 13F, G).

### CONCLUSION

These data support the hypothesis that PU1, IRF8 and BATF3 can reprogram mouse and human cancer cells into cDC1-like cells that can present tumor antigens and induce anti-tumor immune responses. The inventors also validated that reprogramming into cDC1 is conserved across species and tissues and is feasible from primary cancer cells from patients.

### Example 14. Epigenetic Modifiers Enhance cDC1 Reprogramming Efficiency

Given that ectopic expression of PU.1, IRF8 and BATF3 induces cDC1-like fate in unrelated cell types by forcing transcriptional and epigenetic changes, the inventors hypothesized that epigenetic modifiers, namely histone deacetylase inhibition, could enhance the efficiency of cDC1 reprogramming.

### Methods

### ATAC-Seq Library Preparation

To investigate epigenetic changes induced by reprogramming, 5,000-10,000 cells from defined populations were separated via FACS and processed to prepare sequencing library. The quality was assessed using a High Sensitivity DNA Chip (Agilent Technologies), and library was sequenced using a NextSeq 500/550 High Output Kit (150 Cycles) on a NextSeq 500 (Illumina).

### ATAC-Seq Data Analysis

In total, 1,384,592,926 ATAC-seq reads were obtained, with a median sample coverage of approximately 46.7 million reads. To remove Illumina universal adapters NGmerge61 was used by setting adapter-removal mode. Reads were mapped to the GRCh38 reference genome using HISAT2 v2.0.462 with the following parameters: --very-sensitive -k 20. Peak calling was performed with Genrich (v0.6.1, available at https://github.com/jsh58/Genrich, parameters: -m 30 -j -y -r -e chrM) separately for each sample. A combined peak list for all samples was obtained by using PEPATACr R library. Finally, read counts on a combined peak list were calculated with bedtools multicov56. The resulting read counts were processed with R package DESeq252 and normalized using RLE method. PCA was performed using plotPCA function from DESeq2 package. For peak annotation ChIPseeker R library64 was used. To map common chromatin changes, a modified procedure for ATAC-seq data as described for tumour-APC gene expression signature was used. Briefly, for each peak associated with individual genes from the tumour-APC signature, an average difference between day 9 and day 0 was calculated and normalized it to the difference between cDC1 and day 0 for individual phenotype/time point of reprogramming. After that, the inventors took the median of normalised peaks value separately for each phenotype/time point of reprogramming and plotted. For genome tracks, bigwig files were created from bam files with deeptools. Genome tracks were explored using the WashU Epigenome browser. For motif discovery, findMotifsGenome.pl procedure from HOMER67 with default parameters was used on differential ATAC-seq peaks. Functional enrichment analysis for differential ATAC-seq peaks was performed by Great software68 using GO biological process ontology.

### Evaluation of Effect of Epigenetic Modulators in Reprogramming Efficiency

Cancer cell lines were transduced with PIB-IRES-EGFP lentiviral particles or EGFP as control, and cultured in the presence or absence of VPA from reprogramming day 1 to day 4, and reprogramming efficiency was quantified by flow cytometry at reprogramming day 9 in live EGFP⁺ cells according to the surface expression of CD45⁺ and MHC-II or HLA-DR. Reprogrammed cells were then analysed as previously described.

### Results

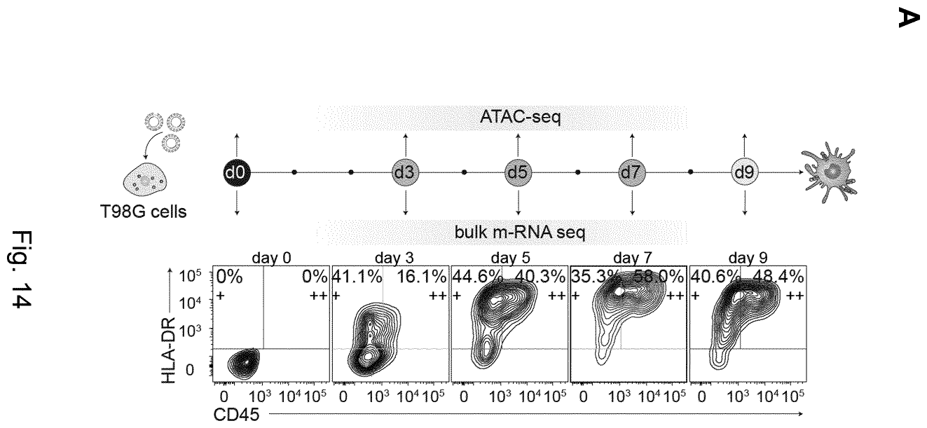

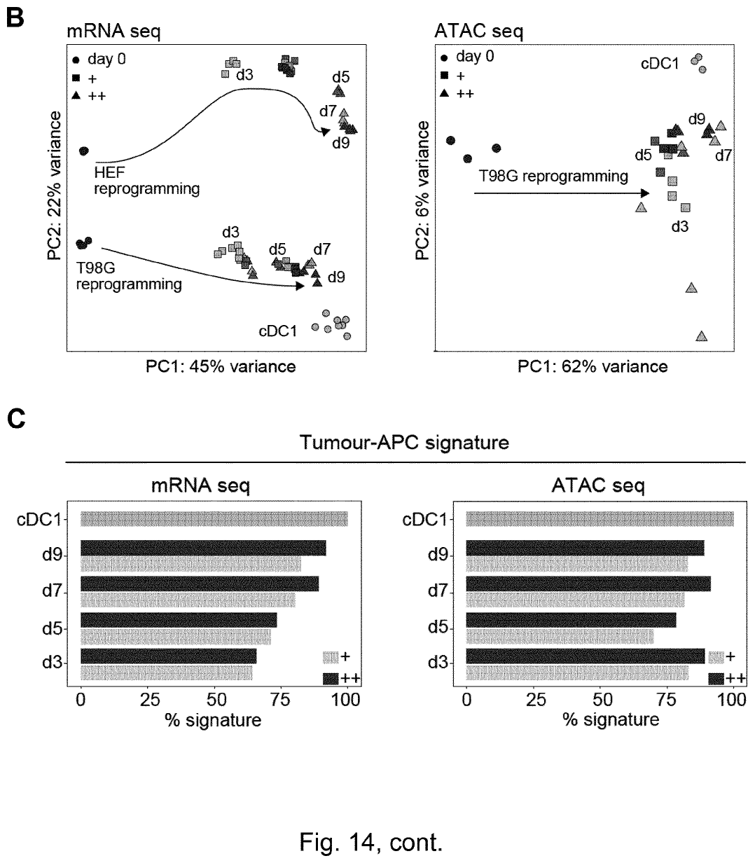

To map the kinetics of human cancer cell reprogramming at the transcriptional and epigenetic levels, reprogrammed (CD45⁺ HLA-DR⁺) and partially reprogrammed (CD45-HLA-DR⁺) T98G cells along a time-course were profiled using mRNA-sequencing and Assay for Transposase accessible chromatin (ATAC) sequencing (FIG. 14A). PCA segregated all reprogramming stages (day 3, 5, 7, and 9) from parental cells, with day 7 and 9 mapping closest to natural cDC1s, indicating a progressive acquisition of cDC1 transcriptional program (FIG. 14B). In agreement, partially reprogrammed cells lagged in the time-course supporting the notion that these cells are on the way to successful reprogramming. Interestingly, reprogramming of human embryonic fibroblasts (HEF) followed a similar reprogramming trajectory (FIG. 14B), indicating that reprogramming dynamics is conserved across malignant and non-cancerous primary cells. PCA for differential open chromatin regions demonstrated that epigenetic remodelling occurred fast with major changes between day 0 and day 3 (62% variance), followed by a fine-tuning at later time points (day 3, 5, 7, 9), bringing cells closer to the open chromatin patterns of cDC1 (FIG. 14B). To confirm these observations, we utilized the tumour-APC gene signature and mapped changes along the time-course. Indeed, the signature was gradually imposed at the transcriptional level and rapidly established at the chromatin level (FIG. 14C). These data show that hPIB-mediated reprogramming elicits rapid epigenetic remodelling followed by a gradual rewiring of the cDC1 transcriptional program.

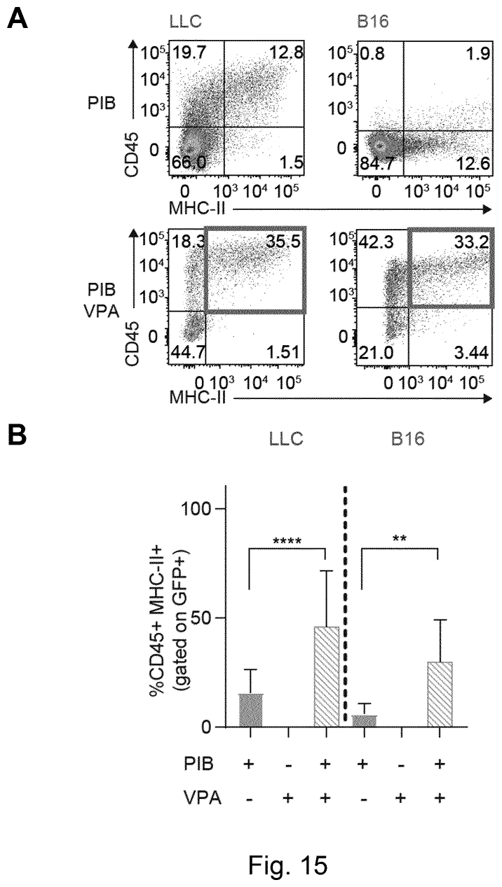

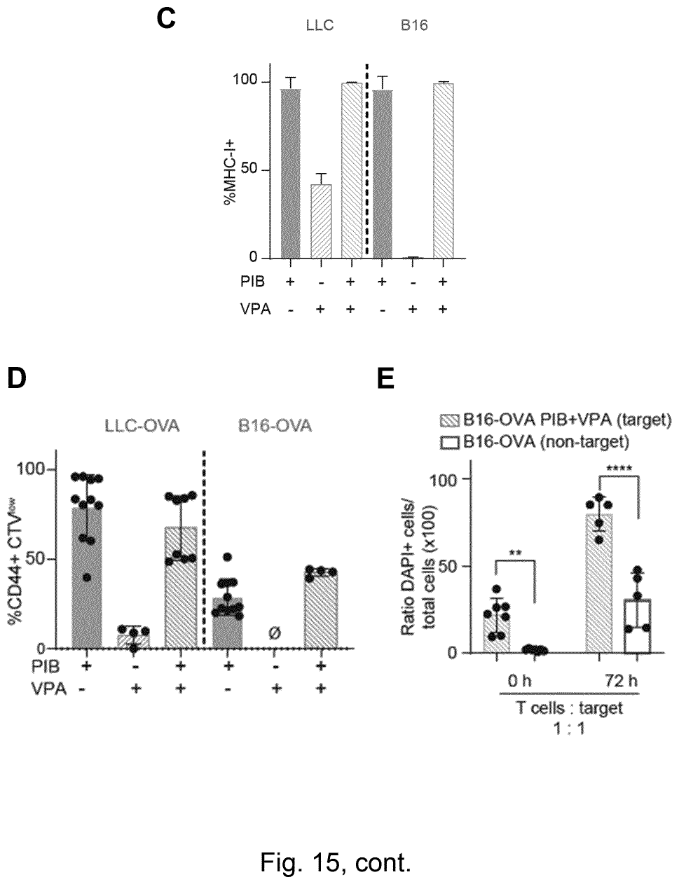

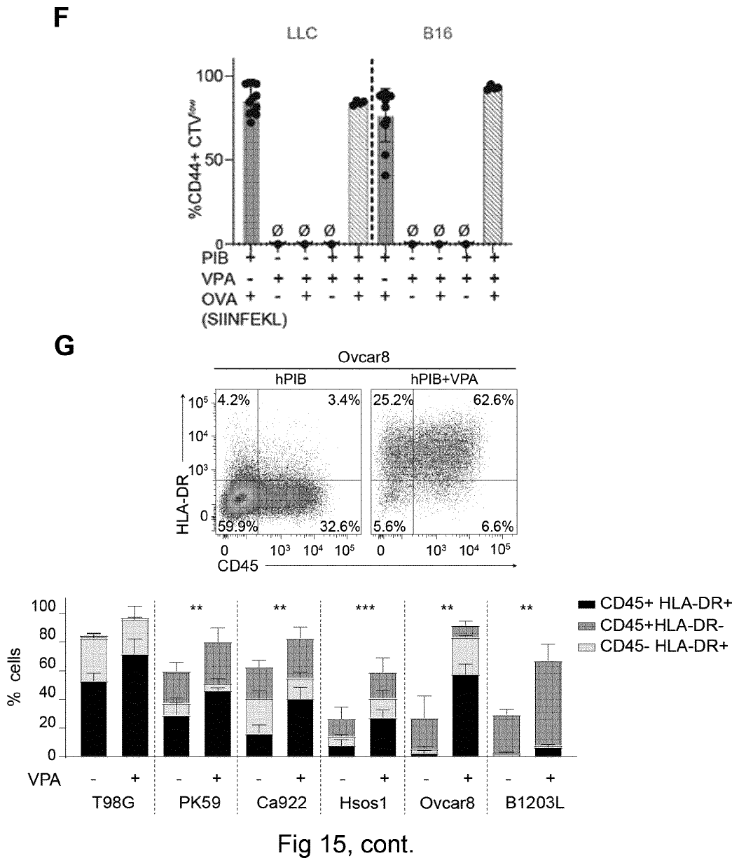

To test whether cDC1 reprogramming efficiency was limited by epigenetic barriers, B16 and LLC cells were treated with Valproic acid (VPA) and assessed reprogramming efficiency at day 9. VPA treatment enhanced the generation of CD45⁺ MHC-II⁺ tumour-APCs by ˜3-fold in LLC (45.9±25.5% vs 15.8±4.79%) and ˜5-fold in B16 (29.9±19.3% vs 5.9±4.8%) cells (FIG. 15A-B). The inventors also confirmed that transduced cells in the presence of VPA upregulate MHC-I (FIG. 15C), indicating that a larger population of cancer cells becomes immunogenic. Functionally, tumour-APCs generated in the presence of VPA presented endogenous antigens to OT-I CD8⁺ T cells (FIG. 15D), became targets of T cell-mediated cytotoxicity (FIG. 15E) and primed naïve CD8⁺ T cells after incubation with exogenous antigens (FIG. 15F). Next, the impact of VPA treatment in cDC1 reprogramming of human cancer cells was investigated. It was observed that VPA treatment increased reprogramming efficiency in all tested lines (FIG. 15G).

### CONCLUSION

This data indicates that reprogramming of cancer cells can be enhanced by facilitating chromatin accessibility during cDC1 reprogramming.

### Example 14. SPIB and SPIC Compensate PU.1 Role in cDC1 Reprogramming

The human genome encodes almost 2000 different transcription factors organized in multiple families and sub-families. Transcription factors that share a significant homology are normally included in the same family/sub-family of transcription factors. Under certain conditions, transcription factors can compensate for the lack of a particular transcription factor from the same family or sub-family. In this regard, the inventors hypothesized that homologues from PU1, IRF8 and BATF3 could compensate their role in cDC1 reprogramming. As a proof-of-concept, the ability of SPIB and SPIC, two PU.1 homologs, to replace the role of PU.1 in cDC1 reprogramming was tested.

### Methods

Coding regions of SPIB and SPIC were individually cloned in the pFUW-TetO plasmid. Lentiviral particles encoding each individual transcription factor or the reverse tetracycline transactivator M2rtTA under the control of constitutively active human ubiquitin C promoter (pFUW-UbC-M2rtTA) were used for co-transduction (Rosa et al. 2018). Reprogramming efficiency was evaluated by flow cytometry in Clec9a-tdTomato mouse embryonic fibroblasts (MEFs) 9 days after transcription factor overexpression.

### Results

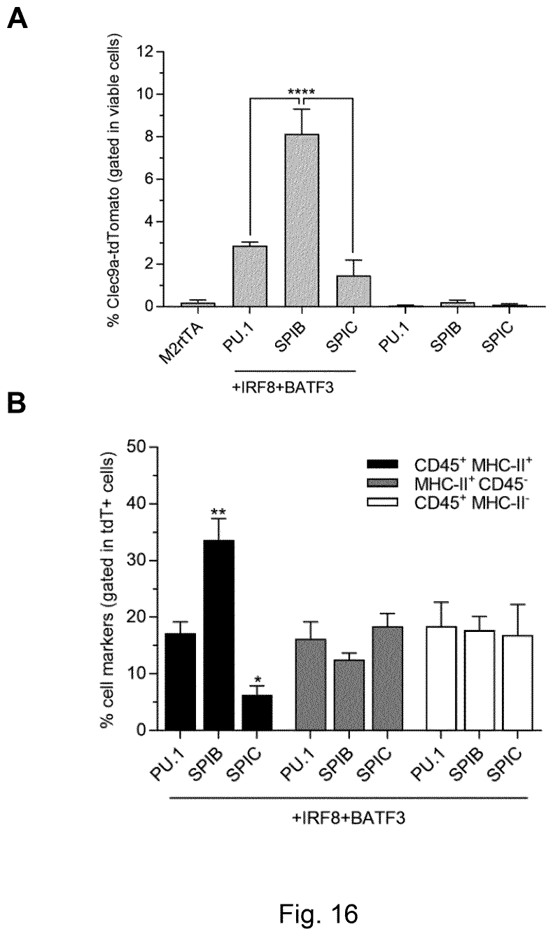

The inventors observed that both SPIB and SPIC could replace PU.1 in the context of cDC1 reprogramming (FIG. 16A). Importantly, SPIB and SPIC alone were not able to activate the DC-specific reporter in transduced MEFs. Interestingly, SPIB induced reporter activation in a greater extent than PU.1, or SPIC, presenting about 8.14±1.16% of tdTomato⁺ cells while PU.1 and SPIC presented only 2.87±0.18% and 1.46±0.73%, respectively.

Next, the expression of CD45 and MHC-II in tdTomato⁺ cells was analyzed. Strikingly, SPIB showed a 2-fold increase in tdTomato⁺ cells co-expressing CD45 and MHC-II (33.63±3.76%) in comparison to PU.1 (17.15±2.04%) (FIG. 16B).

### CONCLUSION

These data suggest that SPIB and SPIC can compensate the role of PU.1 in cDC1 reprogramming.

### Example 15. Delivery of PU.1, IRF8 and BATF3 Mediated by Adenovirus and Adeno-Associated Virus Allows cDC1 Reprogramming of Healthy and Cancer Cells

Cell reprogramming strategies based on the overexpression of cell type-specific transcription factors have traditionally relied on the use of retroviral or lentiviral vectors. Nevertheless, the integrative nature of these viral vectors raises safety concerns for clinical applications. The use of non-integrative viral systems is a good alternative to deliver transcription factors to target cells for therapeutic application bypassing these safety concerns. Here, the inventors hypothesized that delivery of PU.1, IRF8 and BATF3 mediated by non-integrative Adenovirus and Adeno-associated virus (AAVs) allow cDC1 reprogramming in unrelated cell types.

### Methods

### Cell Reprogramming

Mouse embryonic fibroblasts isolated from Clec9a-tdTomato reporter mice, B2905 mouse melanoma cell line, IGR-39 melanoma and T98G Glioblastoma human cell lines, and 2778 human primary melanoma cells were seeded at a density of 12 500 cells/well of 12-well plates and incubated overnight with lentivirus (Lenti), adenovirus (Ad5 or Ad5/F35) or AAVs (AAV-DJ or AAV2-QuadYF) encoding PIB-GFP or GFP only, using the multiplicities of infection 50,000 RNA copies/cell, 5,000 infective units/cell and 250,0000 genomic copies/cell, respectively. When cells were incubated with Lentivirus, media was supplemented with polybrene (8 μg/mL). Media was changed every 2 days for the duration of the cultures. cDC1 reprogramming efficiency was quantified by flow cytometry at reprogramming day 9 in live, GFP⁺ cells according to the surface expression of CD45 and MHC-II or HLA-DR.

### Results

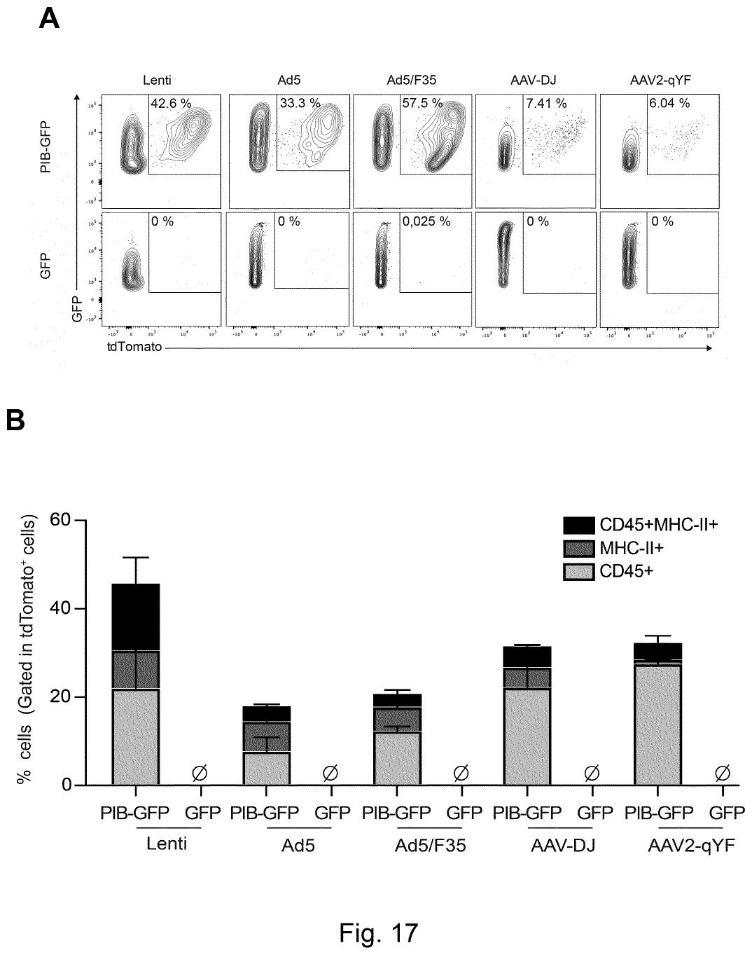

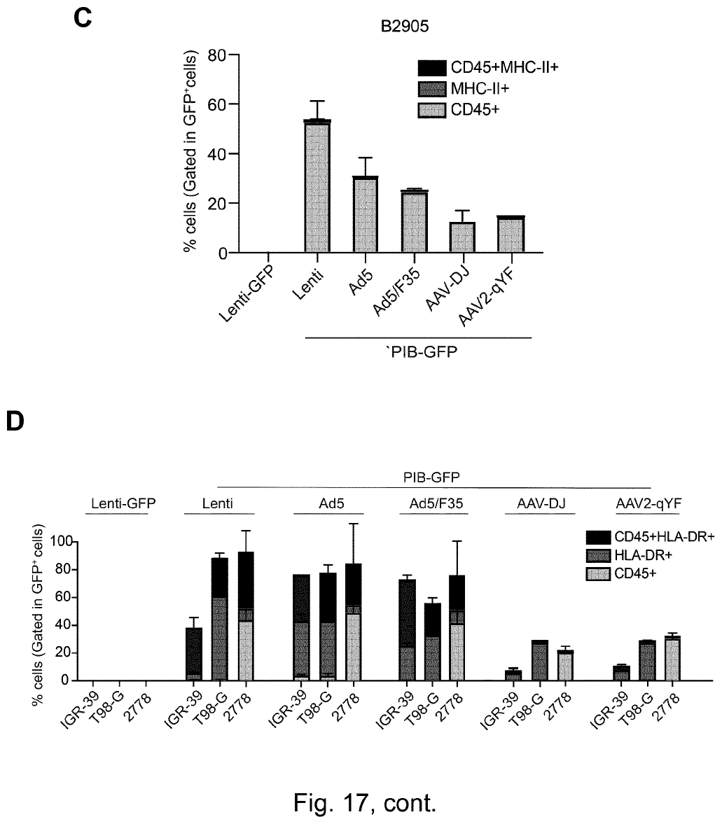

The inventors observed that adenovirus and AAVs encoding PIB-GFP were able to induce activation of the Clec9a-tdTomato reporter (FIG. 17A) and surface expression of CD45 and MHC-II in mouse embryonic fibroblasts (FIG. 17B). As expected, tdTomato expression was not observed in cells transduced with viral vectors encoding GFP. Next, the inventors asked whether Adenovirus and AAV vectors encoding PIB-GFP could reprogram mouse and human cancer cells. Surface expression of CD45 and MHC-II in B2905 mouse melanoma cancer cells (FIG. 17C), and CD45 and HLA-DR in human cancer cell lines (IGR-39 and T98G) and in the human primary melanoma cells 2778 (FIG. 17D) was observed.

### CONCLUSION

These data suggests that delivery of PU.1, IRF8 and BATF3 mediated by Adenovirus and AAVs allows cDC1 reprogramming in healthy and cancerous, mouse and human cell types.

### Sequence Overview

### SEQ ID NO. Description

1 SFFV (Spleen focus-forming virus) promoter polynucleotide sequence
2 MND (myeloproliferative sarcoma virus enhancer, negative control region deleted, dl587rev primer-binding site substituted) promoter polynucleotide sequence
3 CAG (CMV early enhancer/chicken β actin) promoter polynucleotide sequence
4 Cytomegalovirus (CMV) promoter polynucleotide sequence
5 Ubiquitin C (UbC) promoter polynucleotide sequence
6 EF-1 alpha (EF-1α) promoter polynucleotide sequence
7 EF-1 alpha short (EF1S) promoter polynucleotide sequence
8 EF-1 alpha with intron (EF1i) promoter polynucleotide sequence
9 Phosphoglycerate kinase (PGK) promoter polynucleotide sequence
10 Human BATF3 (polypeptide sequence)
11 Human IRF8 isoform 1 (polypeptide sequence)
12 Human PU.1 isoform 1 (polypeptide sequence)
13 Human CCAAT/enhancer-binding protein alpha (CEBPα) (polypeptide sequence)
14 Human BATF3 (polynucleotide sequence)
15 Human IRF8 (polynucleotide sequence)
16 Human PU.1 (polynucleotide sequence)
17 Human CCAAT/enhancer-binding protein alpha (CEBPA) (polynucleotide sequence)
18 Human BATF (polynucleotide sequence)
19 Human BATF (polypeptide sequence)
20 Human IRF7 (polynucleotide sequence)
21 Human IRF7 (polypeptide sequence)
22 Human SPIB (polynucleotide sequence)
23 Human SPIB (polypeptide sequence)
24 Human SPIC (polynucleotide sequence)
25 Human SPIC (polypeptide sequence)

### REFERENCES

J. Alquicira-Hernandez, A. Sathe, H. P. Ji, Q. Nguyen, J. E. Powell, scPred: accurate supervised method for cell-type classification from single-cell RNA-seq data. Genome Biol. 20, 264 (2019).
S. Balan, C. Arnold-Schrauf, A. Abbas, N. Couespel, J. Savoret, F. Imperatore, A. C. Villani, T. P. Vu Manh, N. Bhardwaj, M. Dalod, Large-Scale Human Dendritic Cell Differentiation Revealing Notch-Dependent Lineage Bifurcation and Heterogeneity. Cell reports 24, 1902-1915 e1906 (2018)
K. C. Barry, J. Hsu, M. L. Broz, F. J. Cueto, M. Binnewies, A. J. Combes, A. E. Nelson, K. Loo, R. Kumar, M. D. Rosenblum, M. D. Alvarado, D. M. Wolf, D. Bogunovic, N. Bhardwaj, A. I. Daud, P. K. Ha, W. R. Ryan, J. L. Pollack, B. Samad, S. Asthana, V. Chan, M. F. Krummel, A natural killer-dendritic cell axis defines checkpoint therapy-responsive tumor microenvironments. Nature medicine 24, 1178-1191 (2018).
V. Bergen, M. Lange, S. Peidli, F. A. Wolf, F. J. Theis, Generalizing RNA velocity to transient cell states through dynamical modeling. Nature biotechnology, 38, 1408-1414 (2020).
B. A. Biddy, W. Kong, K. Kamimoto, C. Guo, S. E. Waye, T. Sun, S. A. Morris, Single-cell mapping of lineage and identity in direct reprogramming. Nature. 564, 219-224 (2018).
J. P. Bottcher, E. S. C. Reis, The Role of Type 1 Conventional Dendritic Cells in Cancer Immunity. Trends in cancer 4, 784-792 (2018)
M. L. Broz, M. Binnewies, B. Boldajipour, Amanda E. Nelson, Joshua L. Pollack, David J. Erle, A. Barczak, Michael D. Rosenblum, A. Daud, Diane L. Barber, S. Amigorena, Laura J. van't Veer, Anne I. Sperling, Denise M. Wolf, Matthew F. Krummel, Dissecting the Tumor Myeloid Compartment Reveals Rare Activating Antigen-Presenting Cells Critical for T Cell Immunity. Cancer Cell 26, 638-652 (2014)
J. Cao, M. Spielmann, X. Qiu, X. Huang, D. M. Ibrahim, A. J. Hill, F. Zhang, S. Mundlos, L. Christiansen, F. J. Steemers, C. Trapnell, J. Shendure, The single-cell transcriptional landscape of mammalian organogenesis. Nature 566, 496-502 (2019)
F. Chapuis, M. Rosenzwajg, M. Yagello, M. Ekman, P. Biberfeld, J. C. Gluckman, Differentiation of human dendritic cells from monocytes in vitro. European journal of immunology 27, 431-441 (1997).
M. Dahl, A. Doyle, K. Olsson, J. E. Månsson, A. R. A. Marques, M. Mirzaian, J. M. Aerts, M. Ehinger, M. Rothe, U. Modlich, A. Schambach, S. Karlsson, Lentiviral gene therapy using cellular promoters cures type 1 Gaucher disease in mice. Molecular therapy: the journal of the American Society of Gene Therapy 23, 835-844 (2015).
M. S. Diamond, M. Kinder, H. Matsushita, M. Mashayekhi, G. P. Dunn, J. M. Archambault, H. Lee, C. D. Arthur, J. M. White, U. Kalinke, K. M. Murphy, R. D. Schreiber, Type I interferon is selectively required by dendritic cells for immune rejection of tumours. The Journal of experimental medicine 208, 1989-2003 (2011).
C.-A. Dutertre, E. Becht, S. E. Irac, A. Khalilnezhad, V. Narang, S. Khalilnezhad, P. Y. Ng, L. L. van den Hoogen, J. Y. Leong, B. Lee, M. Chevrier, X. M. Zhang, P. J. A. Yong, G. Koh, J. Lum, S. W. Howland, E. Mok, J. Chen, A. Larbi, H. K. K. Tan, T. K. H. Lim, P. Karagianni, A. G. Tzioufas, B. Malleret, J. Brody, S. Albani, J. van Roon, T. Radstake, E. W. Newell, F. Ginhoux, Single-Cell Analysis of Human Mononuclear Phagocytes Reveals Subset-Defining Markers and Identifies Circulating Inflammatory Dendritic Cells. Immunity. 51, 573-589.e8 (2019).
Ernst, J., Kellis, M. ChromHMM: automating chromatin-state discovery and characterization. Nat Methods 9, 215-216 (2012).
Grajales-Reyes, G., Iwata, A., Albring, J. et al. Batf3 maintains autoactivation of Irf8 for commitment of a CD8α+ conventional DC clonogenic progenitor. Nat Immunol 16, 708-717 (2015).
M. E. Kirkling, U. Cytlak, C. M. Lau, K. L. Lewis, A. Resteu, A. Khodadadi-Jamayran, C. W. Siebel, H. Salmon, M. Merad, A. Tsirigos, M. Collin, V. Bigley, B. Reizis, Notch Signaling Facilitates In vitro Generation of Cross-Presenting Classical Dendritic Cells. Cell reports 23, 3658-3672 e3656 (2018).
G. F. Heidkamp, J. Sander, C. H. K. Lehmann, L. Heger, N. Eissing, A. Baranska, J. J. Lühr, A. Hoffmann, K. C. Reimer, A. Lux, S. Söder, A. Hartmann, J. Zenk, T. Ulas, N. McGovern, C. Alexiou, B. Spriewald, A. Mackensen, G. Schuler, B. Schauf, A. Forster, R. Repp, P. A. Fasching, A. Purbojo, R. Cesnjevar, E. Ullrich, F. Ginhoux, A. Schlitzer, F. Nimmerjahn, J. L. Schultze, D. Dudziak, Human lymphoid organ dendritic cell identity is predominantly dictated by ontogeny, not tissue microenvironment. Sci. Immunol. 1, eaai7677 (2016).
K. Hildner, B. T. Edelson, W. E. Purtha, M. Diamond, H. Matsushita, M. Kohyama, B. Calderon, B. U. Schraml, E. R. Unanue, M. S. Diamond, R. D. Schreiber, T. L. Murphy, K. M. Murphy, Batf3 deficiency reveals a critical role for CD8alpha+ dendritic cells in cytotoxic T cell immunity. Science (New York, N.Y.) 322, 1097-1100 (2008).
K. Honda, H. Yanai, H. Negishi, M. Asagiri, M. Sato, T. Mizutani, N. Shimada, Y. Ohba, A. Takaoka, N. Yoshida, T. Taniguchi, IRF-7 is the master regulator of type-I interferon-dependent immune responses. Nature. 434, 772-777 (2005).
M. Hubert, E. Gobbini, C. Couillault, T.-P. V. Manh, A.-C. Doffin, J. Berthet, C. Rodriguez, V. Ollion, J. Kielbassa, C. Sajous, I. Treilleux, O. Tredan, B. Dubois, M. Dalod, N. Bendriss-Vermare, C. Caux, J. Valladeau-Guilemond, IFN-III is selectively produced by cDC1 and predicts good clinical outcome in breast cancer. Sci. Immunol. 5, eaav3942 (2020).
S. Kim, P. Bagadia, D. Anderson, et al., High Amount of Transcription Factor IRF8 Engages AP1-IRF Composite Elements in Enhancers to Direct Type 1 Conventional Dendritic Cell Identity. Immunity, 53(4), 759-774 (2020).
G. La Manno, R. Soldatov, A. Zeisel, E. Braun, H. Hochgerner, V. Petukhov, K. Lidschreiber, M. E. Kastriti, P. Lonnerberg, A. Furlan, J. Fan, L. E. Borm, Z. Liu, D. van Bruggen, J. Guo, X. He, R. Barker, E. Sundstrom, G. Castelo-Branco, P. Cramer, I. Adameyko, S. Linnarsson, P. V. Kharchenko, RNA velocity of single cells. Nature 560, 494-498 (2018).
H. Lauterbach, B. Bathke, S. Gilles, C. Traidl-Hoffmann, C. A. Luber, G. Fejer, M. A. Freudenberg, G. M. Davey, D. Vremec, A. Kallies, L. Wu, K. Shortman, P. Chaplin, M. Suter, M. O'Keeffe, H. Hochrein, Mouse CD8alpha+ DCs and human BDCA3⁺DCs are major producers of IFN-lambda in response to poly IC. The Journal of experimental medicine 207, 2703-2717 (2010).
H. Li, R. Ghazanfari, D. Zacharaki, N. Ditzel, J. Isern, M. Ekblom, S. Méndez-Ferrer, M. Kassem, S. Scheding, Low/negative expression of PDGFR-α identifies the candidate primary mesenchymal stromal cells in adult human bone marrow. Stem cell reports 3, 965-974 (2014).
M. Mayoux, A. Roller, V. Pulko, S. Sammicheli, S. Chen, E. Sum, C. Jost, M. F. Fransen, R. B. Buser, M. Kowanetz, K. Rommel, I. Matos, S. Colombetti, A. Belousov, V. Karanikas, F. Ossendorp, P. S. Hegde, D. S. Chen, P. Umana, M. Perro, C. Klein, W. Xu, Dendritic cells dictate responses to PD-L1 blockade cancer immunotherapy. Sci. Transl. Med. 12, eaav7431 (2020).
J. Minderjahn, A. Schmidt, A. Fuchs et al. Mechanisms governing the pioneering and redistribution capabilities of the non-classical pioneer PU.1. Nat Commun 11, 402 (2020).
Murphy, T., Tussiwand, R. & Murphy, K. Specificity through cooperation: BATF-IRF interactions control immune-regulatory networks. Nat Rev Immunol 13, 499-509 (2013).
H. A. Pliner, J. Shendure, C. Trapnell, Supervised classification enables rapid annotation of cell atlases. Nature methods 16, 983-986 (2019).
L. F. Poulin, M. Salio, E. Griessinger, F. Anjos-Afonso, L. Craciun, J. L. Chen, A. M. Keller, O. Joffre, S. Zelenay, E. Nye, A. Le Moine, F. Faure, V. Donckier, D. Sancho, V. Cerundolo, D. Bonnet, C. Reis e Sousa, Characterization of human DNGR-1⁺BDCA3⁺leukocytes as putative equivalents of mouse CD8alpha+ dendritic cells. The Journal of experimental medicine 207, 1261-1271 (2010).
C. F. Pires, F. F. Rosa, I. Kurochkin, C.-F. Pereira. Understanding and Modulating Immunity with Cell Reprogramming. Frontiers in Immunology 2019 10: 2809.
F. F. Rosa, C. F. Pires, I. Kurochkin, A. M. Gomes, A. G. Ferreira, L. G. Palma, K. Shaiv, L. Solanas, C. Azenha, D. Papatsenko, O. Schulz, C. Reis e Sousa, C.-F. Pereira, Direct Reprogramming of Fibroblasts into Antigen-Presenting Dendritic Cells. Science immunology 2018 Dec. 7; 3 (30), (2018).
F. F. Rosa, C. F. Pires, O. Zimmermannova, C.-F. Pereira, Direct Reprogramming of Mouse Embryonic Fibroblasts to Conventional Type 1 Dendritic Cells by Enforced Expression of Transcription Factors. Bio-protocol 10, e3619 (2020)
H. Salmon, J. Idoyaga, A. Rahman, M. Leboeuf, R. Remark, S. Jordan, M. Casanova-Acebes, M. Khudoynazarova, J. Agudo, N. Tung, S. Chakarov, C. Rivera, B. Hogstad, M. Bosenberg, D. Hashimoto, S. Gnjatic, N. Bhardwaj, Anna K. Palucka, Brian D. Brown, J. Brody, F. Ginhoux, M. Merad, Expansion and Activation of CD103+ Dendritic Cell Progenitors at the Tumor Site Enhances Tumor Responses to Therapeutic PD-L1 and BRAF Inhibition. Immunity 44, 924-938 (2016).
A. Schambach, J. Bohne, S. Chandra, E. Will, G. P. Margison, D. A. Williams, C. Baum, Equal potency of gammaretroviral and lentiviral SIN vectors for expression of O6-methylguanine-DNA methyltransferase in hematopoietic cells. Molecular Therapy. 13, 391-400 (2006).
P. See, C.-A. Dutertre, J. Chen, P. Günther, N. McGovern, S. E. Irac, M. Gunawan, M. Beyer, K. Händler, K. Duan, H. R. B. Sumatoh, N. Ruffin, M. Jouve, E. Gea-Mallorquí, R. C. M. Hennekam, T. Lim, C. C. Yip, M. Wen, B. Malleret, I. Low, N. B. Shadan, C. F. S. Fen, A. Tay, J. Lum, F. Zolezzi, A. Larbi, M. Poidinger, J. K. Y. Chan, Q. Chen, L. Rénia, M. Haniffa, P. Benaroch, A. Schlitzer, J. L. Schultze, E. W. Newell, F. Ginhoux, Mapping the human DC lineage through the integration of high-dimensional techniques. Science. 356, eaag3009 (2017).
   C. A. Sommer, M. Stadtfeld, G. J. Murphy, K. Hochedlinger, D. N. Kotton, G. Mostoslavsky, Induced Pluripotent Stem Cell Generation Using a Single Lentiviral Stem Cell Cassette. Stem Cells. 27, 543-549 (2009).
S. Sontag, M. Förster, J. Qin, P. Wanek, S. Mitzka, H. M. Schüler, S. Koschmieder, S. Rose-John, K. Seré, M. Zenke, Modelling IRF8 Deficient Human Hematopoiesis and Dendritic Cell Development with Engineered PS Cells. Stem cells (Dayton, Ohio) 35, 898-908 (2017)
S. Spranger, D. Dai, B. Horton, T. F. Gajewski, Tumor-Residing Batf3 Dendritic Cells Are Required for Effector T Cell Trafficking and Adoptive T Cell Therapy. Cancer Cell 31, 711-723.e714 (2017).
Prafullakumar Tailor, Tomohiko Tamura, Herbert C. Morse, Keiko Ozato; The BXH2 mutation in IRF8 differentially impairs dendritic cell subset development in the mouse. Blood 2008; 111(4): 1942-1945.
Y. Tomaru, R. Hasegawa, T. Suzuki, T. Sato, A. Kubosaki, M. Suzuki, H. Kawaji, A. R. R. Forrest, Y. Hayashizaki, FANTOM Consortium, J. W. Shin, H. Suzuki, A transient disruption of fibroblastic transcriptional regulatory network facilitates trans-differentiation. Nucleic Acids Res. 42, 8905-8913 (2014).
B. Treutlein, Q. Y. Lee, J. G. Camp, M. Mall, W. Koh, S. A. M. Shariati, S. Sim, N. F. Neff, J. M. Skotheim, M. Wernig, S. R. Quake, Dissecting direct reprogramming from fibroblast to neuron using single-cell RNA-seq. Nature. 534, 391-395 (2016).
R. Tussiwand, W.-L. Lee, T. L. Murphy, M. Mashayekhi, W. Kc, J. C. Albring, A. T. Satpathy, J. A. Rotondo, B. T. Edelson, N. M. Kretzer, X. Wu, L. A. Weiss, E. Glasmacher, P. Li, W. Liao, M. Behnke, S. S. K. Lam, C. T. Aurthur, W. J. Leonard, H. Singh, C. L. Stallings, L. D. Sibley, R. D. Schreiber, K. M. Murphy, Compensatory dendritic cell development mediated by BATF-IRF interactions. Nature. 490, 502-507 (2012).
A.-C. Villani, R. Satija, G. Reynolds, S. Sarkizova, K. Shekhar, J. Fletcher, M. Griesbeck, A. Butler, S. Zheng, S. Lazo, L. Jardine, D. Dixon, E. Stephenson, E. Nilsson, I. Grundberg, D. McDonald, A. Filby, W. Li, P. L. De Jager, O. Rozenblatt-Rosen, A. A. Lane, M. Haniffa, A. Regev, N. Hacohen, Single-cell RNA-seq reveals new types of human blood dendritic cells, monocytes, and progenitors. Science (New York, N.Y.) 356, (2017).
S. K. Wculek, F. J. Cueto, A. M. Mujal, I. Melero, M. F. Krummel, D. Sancho, Dendritic cells in cancer immunology and immunotherapy. Nature reviews. Immunology, (2019).
Y. Zhou, Z. Liu, J. D. Welch, X. Gao, L. Wang, T. Garbutt, B. Keepers, H. Ma, J. F. Prins, W. Shen, J. Liu, L. Qian, Single-Cell Transcriptomic Analyses of Cell Fate Transitions during Human Cardiac Reprogramming. Cell Stem Cell. 25, 149-164.e9 (2019).

### Items

1. A composition comprising one or more constructs or vectors, which upon expression encodes the transcription factors:
   i) BATF3, or a biologically active variant thereof, wherein the biologically active variant is at least 70% identical to SEQ ID NO: 10 (BATF3), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 10 (BATF3);
   ii) IRF8, or a biologically active variant thereof, wherein the biologically active variant is at least 70% identical to SEQ ID NO: 11 (IRF8), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 11 (IRF8); and
   iii) PU.1, or a biologically active variant thereof, wherein the biologically active variant is at least 70% identical to SEQ ID NO: 12 (PU.1), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 12 (PU.1);
wherein the one or more constructs or vectors comprise a promoter region capable of controlling the transcription of the transcription factors, wherein the promoter region comprises spleen focus-forming virus (SFFV) promoter, MND (myeloproliferative sarcoma virus enhancer, negative control region deleted, dl587rev primer-binding site substituted) promoter, CAG (CMV early enhancer/chicken β actin) promoter, cytomegalovirus (CMV) promoter, ubiquitin C (UbC) promoter, EF-1 alpha (EF-1α) promoter, EF-1 alpha short (EF1S) promoter, EF-1 alpha with intron (EF1i) promoter, phosphoglycerate kinase (PGK) promoter, or a promoter exhibiting essentially the same effect.
2. The composition according to item 1, further comprising one or more constructs or vectors, which upon expression encode one or more transcription factors selected from:
   a) IRF7, or a biologically active variant thereof, wherein the biologically active variant is at least 70% identical to SEQ ID NO: 21 (IRF7) such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 21 (IRF7);
   b) BATF, or a biologically active variant thereof, wherein the biologically active variant is at least 70% identical to SEQ ID NO: 19 (BATF), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 19 (BATF);
   c) SPIB, or a biologically active variant thereof, wherein the biologically active variant is at least 70% identical to SEQ ID NO: 23 (SPIB), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 23 (SPIB);
   d) SPIC, or a biologically active variant thereof, wherein the biologically active variant is at least 70% identical to SEQ ID NO: 25 (SPIC), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 25 (SPIC);

wherein the one or more constructs or vectors comprise a promoter region capable of controlling the transcription of the transcription factors, wherein the promoter region comprises spleen focus-forming virus (SFFV) promoter, MND (myeloproliferative sarcoma virus enhancer, negative control region deleted, dl587rev primer-binding site substituted) promoter, CAG (CMV early enhancer/chicken β actin) promoter, cytomegalovirus (CMV) promoter, ubiquitin C (UbC) promoter, EF-1 alpha (EF-1α) promoter, EF-1 alpha short (EF1S) promoter, EF-1 alpha with intron (EF1i) promoter, phosphoglycerate kinase (PGK) promoter, or a promoter exhibiting essentially the same effect.
3. The composition according to any one of items 1 or 2, wherein:
   a) the SFFV promoter comprises or consists of a polynucleotide sequence at least 80% identical to SEQ ID NO: 1, such as at least 85%, such as at least 90%, such as at least 95%, such as at least 96%, such as at least 97%, such as at least 98%, such as at least 99%, such as 100% identical to SEQ ID NO: 1;
   b) the MND promoter comprises or consists of a polynucleotide sequence at least 80% identical to SEQ ID NO: 2, such as at least 85%, such as at least 90%, such as at least 95%, such as at least 96%, such as at least 97%, such as at least 98%, such as at least 99%, such as 100% identical to SEQ ID NO: 2;
   c) the CAG promoter comprises or consists of a polynucleotide sequence at least 80% identical to SEQ ID NO: 3, such as at least 85%, such as at least 90%, such as at least 95%, such as at least 96%, such as at least 97%, such as at least 98%, such as at least 99%, such as 100% identical to SEQ ID NO: 3;
   d) the CMV promoter comprises or consists of a polynucleotide sequence at least 80% identical to SEQ ID NO: 4, such as at least 85%, such as at least 90%, such as at least 95%, such as at least 96%, such as at least 97%, such as at least 98%, such as at least 99%, such as 100% identical to SEQ ID NO: 4;
   e) the UbC promoter comprises or consists of a polynucleotide sequence at least 80% identical to SEQ ID NO: 5, such as at least 85%, such as at least 90%, such as at least 95%, such as at least 96%, such as at least 97%, such as at least 98%, such as at least 99%, such as 100% identical to SEQ ID NO: 5;
   f) the EF-1α promoter comprises or consists of a polynucleotide sequence at least 80% identical to SEQ ID NO: 6, such as at least 85%, such as at least 90%, such as at least 95%, such as at least 96%, such as at least 97%, such as at least 98%, such as at least 99%, such as 100% identical to SEQ ID NO: 6;
   g) the EF1S promoter comprises or consists of a polynucleotide sequence at least 80% identical to SEQ ID NO: 7, such as at least 85%, such as at least 90%, such as at least 95%, such as at least 96%, such as at least 97%, such as at least 98%, such as at least 99%, such as 100% identical to SEQ ID NO: 7;
   h) the EF1i promoter comprises or consists of a polynucleotide sequence at least 80% identical to SEQ ID NO: 8, such as at least 85%, such as at least 90%, such as at least 95%, such as at least 96%, such as at least 97%, such as at least 98%, such as at least 99%, such as 100% identical to SEQ ID NO: 8; and
   i) the PGK promoter comprises or consists of a polynucleotide sequence at least 80% identical to SEQ ID NO: 9, such as at least 85%, such as at least 90%, such as at least 95%, such as at least 96%, such as at least 97%, such as at least 98%, such as at least 99%, such as 100% identical to SEQ ID NO: 9.
4. The composition according to any one of the preceding items, wherein the composition comprises:
   a) one construct or vector which upon expression encodes the transcription factors BATF3, IRF8 and PU.1;
   b) one construct or vector which upon expression encodes the transcription factors BATF3, IRF8 and SPIB;
   c) a first construct or vector which upon expression encodes the transcription factors BATF3 and IRF8, and a second construct or vector which upon expression encodes the transcription factor PU.1;
   d) a first construct or vector which upon expression encodes the transcription factors BATF3 and IRF8, and a second construct or vector which upon expression encodes the transcription factor SPIB;
   e) a first construct or vector which upon expression encodes the transcription factor BATF3, and a second construct or vector which upon expression encodes the transcription factors IRF8 and PU.1;
   f) a first construct or vector which upon expression encodes the transcription factor BATF3, and a second construct or vector which upon expression encodes the transcription factors IRF8 and SPIB;
   g) a first construct or vector which upon expression encodes the transcription factor IRF8, and a second construct or vector which upon expression encodes the transcription factors BATF3 and PU.1;
   h) a first construct or vector which upon expression encodes the transcription factor IRF8, and a second construct or vector, which upon expression encodes the transcription factors BATF3 and SPIB;
   i) a first construct or vector which upon expression encodes the transcription factor BATF3; a second construct or vector which upon expression encodes the transcription factor IRF8; and a third construct or vector which upon expression encodes the transcription factor PU.1; and/or
   j) a first construct or vector which upon expression encodes the transcription factor BATF3, a second construct or vector which upon expression encodes the transcription factor IRF8, and a third construct or vector which upon expression encodes the transcription factor SPIB.
5. The composition according to any one of the preceding items, wherein the composition comprises:
   a) one construct or vector which upon expression encodes the transcription factors BATF3, IRF8, PU.1 and IRF7;
   b) a first construct or vector which upon expression encodes the transcription factors BATF3 and IRF8, and a second construct or vector which upon expression encodes the transcription factors PU.1 and IRF7;
   c) a first construct or vector which upon expression encodes the transcription factors BATF3 and PU.1, and a second construct or vector which upon expression encodes the transcription factors IRF8 and IRF7;
   d) a first construct or vector which upon expression encodes the transcription factors PU.1 and IRF8, and a second construct or vector which upon expression encodes the transcription factors BATF3 and IRF7;
   e) a first construct or vector which upon expression encodes the transcription factors BATF3, IRF8 and PU.1, and a second construct or vector which upon expression encodes the transcription factor IRF7;
   f) a first construct or vector which upon expression encodes the transcription factor BATF3, and a second construct or vector which upon expression encodes the transcription factors IRF8, PU.1 and IRF7;
   g) a first construct or vector which upon expression encodes the transcription factor IRF8, and a second construct or vector which upon expression encodes the transcription factors BATF3, PU.1 and IRF7;
   h) a first construct or vector which upon expression encodes the transcription factor PU.1, and a second construct or vector which upon expression encodes the transcription factors BATF3, IRF8 and IRF7; and/or
   i) a first construct or vector which upon expression encodes the transcription factor BATF3; a second construct or vector which upon expression encodes the transcription factor IRF8; a third construct or vector which upon expression encodes the transcription factor PU.1 and a fourth construct or vector which upon expression encodes the transcription factor IRF7;
6. The composition according to any one of the preceding items, wherein the composition comprises:
   a) one construct or vector which upon expression encodes the transcription factors BATF3, IRF8, PU.1 and BATF;
   b) a first construct or vector which upon expression encodes the transcription factors BATF3 and IRF8, and a second construct or vector which upon expression encodes the transcription factors PU.1 and BATF;
   c) a first construct or vector which upon expression encodes the transcription factors BATF3 and PU.1, and a second construct or vector which upon expression encodes the transcription factors IRF8 and BATF;
   d) a first construct or vector which upon expression encodes the transcription factors PU.1 and IRF8, and a second construct or vector which upon expression encodes the transcription factors BATF3 and BATF;
   e) a first construct or vector which upon expression encodes the transcription factors BATF3, IRF8 and PU.1, and a second construct or vector which upon expression encodes the transcription factor BATF;
   f) a first construct or vector which upon expression encodes the transcription factor BATF3, and a second construct or vector which upon expression encodes the transcription factors IRF8, PU.1 and BATF;
   g) a first construct or vector which upon expression encodes the transcription factor IRF8, and a second construct or vector which upon expression encodes the transcription factors BATF3, PU.1 and BATF;
   h) a first construct or vector which upon expression encodes the transcription factor PU.1, and a second construct or vector which upon expression encodes the transcription factors BATF3, IRF8 and BATF; and/or
   i) a first construct or vector which upon expression encodes the transcription factor BATF3; a second construct or vector which upon expression encodes the transcription factor IRF8; a third construct or vector which upon expression encodes the transcription factor PU.1 and a fourth construct or vector which upon expression encodes the transcription factor BATF;
7. The composition according to any one of the preceding items, wherein BATF3 is encoded by a polynucleotide sequence with at least 90% sequence identity to SEQ ID NO: 14, such as at least 95%, such as at least 96%, such as at least 97%, such as at least 98%, such as at least 99%, such as 100% sequence identity to SEQ ID NO: 14.
8. The composition according to any one of the preceding items, wherein IRF8 is encoded by a polynucleotide sequence with at least 90% sequence identity to SEQ ID NO: 15, such as at least 95%, such as at least 96%, such as at least 97%, such as at least 98%, such as at least 99%, such as 100% sequence identity to SEQ ID NO: 15.
9. The composition according to any one of the preceding items, wherein PU.1 is encoded by a polynucleotide sequence with at least 90% sequence identity to SEQ ID NO: 16, such as at least 95%, such as at least 96%, such as at least 97%, such as at least 98%, such as at least 99%, such as 100% sequence identity to SEQ ID NO: 16.
10. The composition according to any one of the preceding items, wherein BATF is encoded by a polynucleotide sequence with at least 90% sequence identity to SEQ ID NO: 18, such as at least 95%, such as at least 96%, such as at least 97%, such as at least 98%, such as at least 99%, such as 100% sequence identity to SEQ ID NO: 18.
11. The composition according to any one of the preceding items, wherein IRF7 is encoded by a polynucleotide sequence with at least 90% sequence identity to SEQ ID NO: 20, such as at least 95%, such as at least 96%, such as at least 97%, such as at least 98%, such as at least 99%, such as 100% sequence identity to SEQ ID NO: 20.
12. The composition according to any one of the preceding items, wherein SPIB is encoded by a polynucleotide sequence with at least 90% sequence identity to SEQ ID NO: 22, such as at least 95%, such as at least 96%, such as at least 97%, such as at least 98%, such as at least 99%, such as 100% sequence identity to SEQ ID NO: 22.
13. The composition according to any one of the preceding items, wherein SPIC is encoded by a polynucleotide sequence with at least 90% sequence identity to SEQ ID NO: 24, such as at least 95%, such as at least 96%, such as at least 97%, such as at least 98%, such as at least 99%, such as 100% sequence identity to SEQ ID NO: 24.
14. The composition according to any one of the preceding items, wherein the one or more constructs or vectors upon expression further encodes the transcription factor CCAAT/enhancer-binding protein alpha (CEBPα), or a biologically active variant thereof, wherein the biologically active variant is at least 70% identical to SEQ ID NO: 13 (CEBPα), such as at least 75%, such as at least 80%, such as at least 85%, such as at least 90%, such as at least 95%, such as at least 96%, such as at least 97%, such as at least 98%, such as at least 99%, such as 100% identical to SEQ ID NO: 13.
15. The composition according to any one of the preceding items, wherein CEBPα is encoded by a polynucleotide sequence with at least 90% sequence identity to SEQ ID NO: 17, such as at least 95%, such as at least 96%, such as at least 97%, such as at least 98%, such as at least 99%, such as 100% sequence identity to SEQ ID NO: 17.
16. The composition according to any one of the preceding items, wherein the one or more constructs or vectors further comprise self-cleaving peptides operably linked to at least two of the at least three coding regions, thus forming a single open reading frame.
17. The composition according to item 16, wherein the self-cleaving peptides are 2A peptides.
18. The composition according to item 17, wherein the 2A peptides are selected from the group consisting of equine rhinitis A virus (E2A), foot-and-mouth disease virus (F2A), porcine teschovirus-1 (P2A) and Thosea signa virus (T2A) peptides.
19. The composition according to any one of the preceding items, wherein the one or more constructs or vectors is a viral vector.
20. The composition according to any one of the preceding items, wherein the one or more constructs or vectors is a viral vector selected from the group consisting of: lentiviral vectors, retrovirus vectors, adenovirus vectors, herpes virus vectors, pox virus vectors, adeno-associated virus vectors, paramyxoviridae vectors, rabdoviral vectors, alphaviral vectors and flaviral vectors.
21. The composition according to item 20, wherein the viral vector is a lentiviral vector
22. The composition according to item 20, wherein the adenovirus vector is selected from the group consisting of: wild-type Ad vectors, hybrid Ad vectors and mutant Ad vectors.
23. The composition according to item 22, wherein the wild-type Ad vectors is Ad5 and wherein the hybrid Ad vector is Ad5/F35.
24. The composition according to item 20, wherein the adeno-associated virus vector is selected from the group consisting of: wild-type AAV vectors, hybrid AAV vectors and mutant AAV vectors.
25. The composition according to item 24, wherein the hybrid AAV vector is AAV-DJ and wherein the mutant AAV vector is AAV2-QuadYF.
26. The composition according to any one of the preceding items, wherein the one or more constructs or vectors is a plasmid.
27. The composition according to any one of the preceding items, wherein the backbone of the one or more constructs or vectors is selected from the group consisting of: FUW, pRRL-cPPT, pRLL, pCCL, pCLL, pHAGE2, pWPXL, pLKO, pHIV, pLL, pCDH and pLenti.
28. The composition according to any one of the preceding items, wherein the one or more constructs or vectors further comprise a posttranscriptional regulatory element (PRE) sequence.
29. The composition according to item 28, wherein the PRE sequence is a Woodchuck hepatitis virus posttranscriptional regulatory element (WPRE).
30. The composition according to any one of the preceding items, wherein the one or more constructs or vectors further comprise a central polypurine tract (cPPT).
31. The composition according to any one of the preceding items, wherein the one or more constructs or vectors further comprise 5′ and 3′ terminal repeats.
32. The composition according to item 31, wherein at least one of the 5′ and 3′ terminal repeats is a lentiviral long terminal repeat or a self-inactivating (SIN) design with partially deleted U3 of the 3′ long terminal repeat.
33. The composition according to any one of the preceding items, wherein the one or more constructs or vectors further comprise a nucleocapsid protein packaging target site.
34. The composition according to item 33, wherein the protein packaging target site comprises a HIV-1 psi sequence.
35. The composition according to any one of the preceding items, wherein the one or more constructs or vectors further comprise a REV protein response element (RRE).
36. The composition according to any one of the preceding items, further comprising one or more cytokines selected from the group consisting of: IFNβ, IFNγ, TNFα, IFNα, IL-1β, IL-6, CD40I, Flt3I, GM-CSF, IFN-λ1, IFN-ω, IL-2, IL-4, IL-15, prostaglandin 2, SCF and oncostatin M (OM).
37. The composition according to any one of the preceding items, further comprising one or more epigenetic modifiers, such as histone deacetylase inhibitors.
38. The composition according to item 37, wherein the one or more histone deacetylase inhibitor is valproic acid.
39. The composition according to any one of the preceding items, wherein the composition is a pharmaceutical composition.
40. A cell comprising one or more constructs or vectors according to any one of the preceding items.
41. The cell according to item 40, wherein the cell is a mammalian cell, such as a human or a murine cell.
42. The cell according to any one of items 40 to 41, wherein the cell is selected from the group consisting of: a stem cell, a differentiated cell, and a cancer cell, wherein:
   a) the stem cell is selected from the group consisting of: a pluripotent stem cell and a multipotent stem cell, such as a mesenchymal stem cell or a hematopoietic stem cell;
   b) the differentiated cell is any somatic cell, such as a fibroblast or a hematopoietic cell, such as a monocyte.
43. The cell according to any one of items 40 to 42, wherein the cell is a reprogrammed human dendritic or antigen-presenting cell, such as a human type 1 conventional dendritic cell.
44. The cell according to any one of items 40 to 43, wherein the cell further expresses one or more surface marker(s) selected from the surface markers of table 1.
45. The cell according to any one of items 40 to 44, wherein the cell is positive for one or more surface markers listed in table 1.
46. The cell according to any one of items 40 to 45, wherein the cell is CD226 positive.
47. A method of reprogramming or inducing a cell into a dendritic or antigen-presenting cell, the method comprising the following steps:
   c) transducing a cell with a composition comprising a construct or vector according to any one of items 1 to 32.
   d) expressing the transcription factors;
whereby a reprogrammed or induced cell is obtained.
48. The method according to item 47, wherein the reprogramming or induction is in vivo, in vitro, or ex vivo.
49. The method according to any one of items 47 to 48, wherein the method further comprises a step of culturing the transduced cell in a cell media, wherein the step is conducted before or after expressing the transcription factors.
50. The method according to any one of items 47 to 49, wherein the method further comprises culturing the transduced cell in a cell media comprising one or more cytokines selected form the group consisting of: IFNβ, IFNγ, TNFα, IFNα, IL-1β, IL-6, CD40I, Flt3I, GM-CSF, IFN-λ1, IFN-ω, IL-2, IL-4, IL-15, prostaglandin 2, SCF and oncostatin M (OM).
51. The method according to any one of items 47 to 50, wherein the method further comprises culturing the transduced cell in a cell media comprising one or more epigenetic modifiers, such as histone deacetylase inhibitors.
52. The method according to item 51, wherein the histone deacetylase inhibitor is valproic acid.
53. The method according to any one of items 47 to 52, wherein the cell is a mammalian cell, such as a human or a murine cell.
54. The method according to any one of items 47 to 53, wherein the cell is selected from the group consisting of: a stem cell, a differentiated cell, and a cancer cell, wherein:
   e) the stem cell is selected from the group consisting of: a pluripotent stem cell and a multipotent stem cell, such as a mesenchymal stem cell or a hematopoietic stem cell;
   f) the differentiated cell is any somatic cell, such as a fibroblast or a hematopoietic cell such as a monocyte.
55. The method according to any one of items 47 to 54, wherein the transduced cell is cultured during at least 2 days, such as at least 5 days, such as at least 8 days, such as at least 10 days, such as at least 12 days.
56. The method according to any one of items 47 to 55, wherein the resulting reprogrammed or induced cell is a type 1 conventional dendritic cell.
57. The method according to any one of items 47 to 56, wherein the resulting reprogrammed or induced cell is cluster differentiation 45 (CD45) positive.
58. The method according to any one of items 47 to 57, wherein the resulting reprogrammed or induced cell is X-C Motif Chemokine Receptor 1 (XCR1) positive.
59. The method according to any one of items 47 to 58, wherein the resulting reprogrammed or induced cell is cluster differentiation 226 (CD226) positive.
60. The method according to any one of items 47 to 59, wherein the resulting reprogrammed or induced cell is human leukocyte antigen-DR isotype (HLA-DR) positive.
61. A reprogrammed or induced cell obtained according to the method defined in any one of items 47 to 60.
62. The reprogrammed or induced cell according to item 61, wherein the cell is a dendritic or antigen-presenting cell, such as a type 1 conventional dendritic cell.
63. The reprogrammed or induced cell according to any one of items 61 to 62 wherein the resulting reprogrammed or induced cell is positive for one or more surface markers listed in table 1.
64. The reprogrammed or induced cell according to any one of items 61 to 63, wherein the resulting reprogrammed or induced cell is CD45, HLA-DR, CD141, CLEC9A, XCR1 and/or CD226 positive.
65. The composition according to any one of items 1 to 39, the cell according to any one of items 40 to 46, and/or the reprogrammed or induced cell according to any one of items 61 to 64, for use in medicine.
66. The composition according to any one of items 1 to 39, the cell according to any one of items 40 to 46, and/or the reprogrammed or induced cell according to any one of items 61 to 64, for use in the treatment of cancer or infectious diseases.
67. The composition, the cell and/or the reprogrammed cell according to item 66, wherein the cancer is selected from the group consisting of: basal cell carcinoma, cervical dysplasia, sarcoma, germ cell tumor, retinoblastoma, glioblastoma, lymphoma, Hodgkin's lymphoma, non-Hodgkin's lymphoma, blood cancer, prostate cancer, ovarian cancer, cervix cancer, oesophageal cancer, uterus cancer, vaginal cancer, breast cancer, head and neck cancer, gastric cancer, oral cavity cancer, naso-pharynx cancer, trachea cancer, larynx cancer, bronchi cancer, bronchioles cancer, lung cancer, pleural cancer, bladder and urothelial cancer, hollow organs cancer, esophagus cancer, stomach cancer, bile duct cancer, intestine cancer, colon cancer, colorectum cancer, rectum cancer, bladder cancer, ureter cancer, kidney cancer, liver cancer, gall bladder cancer, spleen cancer, brain cancer, lymphatic system cancer, bone cancer, pancreatic cancer, leukemia, chronic myeloid leukemia, acute lymphoblastic leukemia, acute myeloid leukemia, skin cancer, melanoma and myeloma, preferably wherein the cancer is selected from the group consisting of: melanoma, head and neck cancer, breast cancer, colorectal cancer, liver cancer, lymphoma, bladder and urothelial cancer, pancreatic cancer and glioblastoma.
68. A method of treating cancer or infectious diseases, the method comprising administering to an individual in need thereof the composition according to any one of items 1 to 38, the cell according to any one of items 40 to 46, the pharmaceutical composition according to item 39, and/or the reprogrammed or induced cell according to any one of items 61 to 64.
69. Use of the composition according to any one of items 1 to 38, the cell according to any one of items 40 to 46, the pharmaceutical composition according to item 39, and/or the reprogrammed or induced cell according to any one of items 61 to 64, for the manufacture of a medicament for the treatment of cancer or infectious disease.

1-69. (canceled)

70. A composition comprising one or more constructs or vectors, which upon expression encodes the transcription factors:
a) BATF3, or a biologically active variant thereof, wherein the biologically active variant is at least 90% identical to SEQ ID NO: 10;
b) IRF8, or a biologically active variant thereof, wherein the biologically active variant is at least 90% identical to SEQ ID NO: 11; and
c) PU.1, or a biologically active variant thereof, wherein the biologically active variant is at least 90% identical to SEQ ID NO: 12;
wherein the one or more constructs or vectors comprise a promoter region capable of controlling the transcription of the transcription factors, wherein the promoter region comprises the spleen focus-forming virus (SFFV) promoter.

71. The composition according to claim 70, further comprising one or more constructs or vectors, which upon expression encode one or more transcription factors selected from:
a) IRF7, or a biologically active variant thereof, wherein the biologically active variant is at least 90% identical to SEQ ID NO: 21;
b) BATF, or a biologically active variant thereof, wherein the biologically active variant is at least 90% identical to SEQ ID NO: 19;
c) SPIB, or a biologically active variant thereof, wherein the biologically active variant is at least 90% identical to SEQ ID NO: 23; and
d) SPIC, or a biologically active variant thereof, wherein the biologically active variant is at least 90% identical to SEQ ID NO: 25;
wherein the one or more constructs or vectors comprise a promoter region capable of controlling the transcription of the transcription factors, wherein the promoter region comprises spleen focus-forming virus (SFFV) promoter.

72. The composition according to claim 70, wherein the SFFV promoter comprises or consists of a polynucleotide sequence at least 90% identical to SEQ ID NO: 1.

73. The composition according to claim 70, wherein the composition comprises:
a) one construct or vector which upon expression encodes the transcription factors BATF3, IRF8 and PU.1;
b) one construct or vector which upon expression encodes the transcription factors BATF3, IRF8 and SPIB;
c) a first construct or vector which upon expression encodes the transcription factors BATF3 and IRF8, and a second construct or vector which upon expression encodes the transcription factor PU.1;
d) a first construct or vector which upon expression encodes the transcription factors BATF3 and IRF8, and a second construct or vector which upon expression encodes the transcription factor SPIB;
e) a first construct or vector which upon expression encodes the transcription factor BATF3, and a second construct or vector which upon expression encodes the transcription factors IRF8 and PU.1;
f) a first construct or vector which upon expression encodes the transcription factor BATF3, and a second construct or vector which upon expression encodes the transcription factors IRF8 and SPIB;
g) a first construct or vector which upon expression encodes the transcription factor IRF8, and a second construct or vector which upon expression encodes the transcription factors BATF3 and PU.1;
h) a first construct or vector which upon expression encodes the transcription factor IRF8, and a second construct or vector, which upon expression encodes the transcription factors BATF3 and SPIB;
i) a first construct or vector which upon expression encodes the transcription factor BATF3; a second construct or vector which upon expression encodes the transcription factor IRF8; and a third construct or vector which upon expression encodes the transcription factor PU.1;
and/or
j) a first construct or vector which upon expression encodes the transcription factor BATF3, a second construct or vector which upon expression encodes the transcription factor IRF8, and a third construct or vector which upon expression encodes the transcription factor SPIB.

74. The composition according to claim 70, wherein the one or more constructs or vectors are lentiviral vectors, retrovirus vectors, adenovirus vectors, herpes virus vectors, pox virus vectors, adeno-associated virus vectors, paramyxoviridae vectors, rabdoviral vectors, alphaviral vectors and/or flaviral vectors.

75. The composition according to claim 74, wherein the adenovirus vector is a wild-type Ad vector, hybrid Ad vector and/or mutant Ad vector.

76. The composition according to claim 75, wherein the wild-type Ad vector is Ad5 and wherein the hybrid Ad vector is Ad5/F35.

77. A cell comprising one or more constructs or vectors according to claim 70.

78. The cell according to claim 77, wherein the cell is a mammalian cell.

79. The cell according claim 77, wherein the cell is a stem cell, a differentiated cell, or a cancer cell, wherein:
a) the stem cell is a pluripotent stem cell or a multipotent stem cell; and
b) the differentiated cell is any somatic cell.

80. A method of reprogramming or inducing a cell into a dendritic or antigen-presenting cell, the method comprising the following steps:
a) transducing a cell with a composition comprising a construct or vector according to any one of claim 70; and
b) expressing the transcription factors;
whereby a reprogrammed or induced cell is obtained.

81. The method according to claim 80, wherein the reprogramming or induction is in vivo, in vitro, or ex vivo.

82. The method according to claim 80, wherein the method further comprises a step of culturing the transduced cell in a cell media, wherein the step is conducted before or after expressing the transcription factors.

83. The method according to claim 80, wherein the method further comprises culturing the transduced cell in a cell media comprising one or more epigenetic modifiers, and wherein the epigenetic modifiers are histone deacetylase inhibitors.

84. The method according to claim 83, wherein the histone deacetylase inhibitor is valproic acid.

85. A reprogrammed or induced cell obtained according to the method defined in claim 80.

86. The reprogrammed or induced cell according to claim 85, wherein the cell is a type 1 conventional dendritic cell.

87. The reprogrammed or induced cell according to claim 85, wherein the resulting reprogrammed or induced cell is CD45, HLA-DR, CD141, CLEC9A, XCR1 and/or CD226 positive.

88. A method of treating cancer or infectious diseases comprising administering a therapeutically effective amount of the composition according to claim 70 to a subject in need thereof.

89. The method according to claim 88, wherein the cancer is selected from the group consisting of: melanoma, head and neck cancer, colorectal cancer, basal cell carcinoma, cervical dysplasia, sarcoma, germ cell tumor, retinoblastoma, glioblastoma, lymphoma, Hodgkin's lymphoma, non-Hodgkin's lymphoma, blood cancer, prostate cancer, ovarian cancer, cervix cancer, oesophageal cancer, uterus cancer, vaginal cancer, breast cancer, gastric cancer, oral cavity cancer, naso-pharynx cancer, trachea cancer, larynx cancer, bronchi cancer, bronchioles cancer, lung cancer, pleural cancer, urothelial cancer, hollow organs cancer, esophagus cancer, stomach cancer, bile duct cancer, intestine cancer, colon cancer, rectum cancer, bladder cancer, ureter cancer, kidney cancer, liver cancer, gall bladder cancer, spleen cancer, brain cancer, lymphatic system cancer, bone cancer, pancreatic cancer, leukemia, chronic myeloid leukemia, acute lymphoblastic leukemia, acute myeloid leukemia, skin cancer, and myeloma.


SEQUENCE LISTING (25 sequences)

SEQ ID NO: 1 - DNA, 406 bp, Artificial
  SFFV (Spleen focus-forming virus) promoter sequence
  misc_feature: SFFV (Spleen focus-forming virus) promoter sequence
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcggccg

SEQ ID NO: 2 - DNA, 596 bp, Artificial
  MND (myeloproliferative sarcoma virus enhancer, negative control region deleted, dl587rev primer-binding site substituted) promoter sequence
  misc_feature: MND (myeloproliferative sarcoma virus enhancer, negative control region deleted, dl587rev primer-binding site substituted) promoter sequence
cgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatc

SEQ ID NO: 3 - DNA, 584 bp, Artificial
  CAG (CMV early enhancer/chicken beta actin) promoter sequence
  misc_feature: CAG (CMV early enhancer/chicken beta actin) promoter sequence
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - DNA, 508 bp, Artificial
  Cytomegalovirus (CMV) promoter sequence
  misc_feature: Cytomegalovirus (CMV) promoter sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 5 - DNA, 1212 bp, Homo sapiens
  misc_feature: Ubiquitin C (UbC) promoter sequence
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 6 - DNA, 231 bp, Artificial
  EF-1 alpha (EF-1alpha) promoter sequence
  misc_feature: EF-1 alpha (EF-1alpha) promoter sequence
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 7 - DNA, 212 bp, Artificial
  EF-1 alpha short (EF1S) promoter sequence
  misc_feature: EF-1 alpha short (EF1S) promoter sequence
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 8 - DNA, 1179 bp, Artificial
  EF-1 alpha with intron (EF1i) promoter sequence
  misc_feature: EF-1 alpha with intron (EF1i) promoter sequence
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - DNA, 505 bp, Homo sapiens
  misc_feature: Phosphoglycerate kinase (PGK) promoter sequence
gggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 10 - protein, 126 aa, Homo sapiens
  MISC_FEATURE: Human BATF3 (protein sequence)
MSQGLPAAGSVLQRSVAAPGNQPQPQPQQQSPEDDDRKVRRREKNRVAAQRSRKQTQKADKLHEEYESLEQENTMLRREIGKLTEELKHLTEALKEHEKMCPLLLCPMNFVPVPPRPDPVAGCLPR

SEQ ID NO: 11 - protein, 425 aa, Homo sapiens
  MISC_FEATURE: Human IRF8 isoform 1 (protein sequence)
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 12 - protein, 271 aa, Homo sapiens
  MISC_FEATURE: Human PU.1 isoform 1 (protein sequence)
MLQACKMEGFPLVPPQPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 13 - protein, 239 aa, Homo sapiens
  MISC_FEATURE: Human CCAAT/enhancer-binding protein alpha (CEBPalpha) (protein sequence)
MPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 14 - DNA, 384 bp, Homo sapiens
  misc_feature: Human BATF3 (DNA sequence)
atgtcgcaagggctcccggccgccggcagcgtcctgcagaggagcgtcgcggcgcccgggaaccagccgcagccgcagccgcagcagcagagccctgaggatgatgacaggaaggtccgaaggagagaaaaaaaccgagttgctgctcagagaagtcggaagaagcagacccagaaggctgacaagctccatgaggaatatgagagcctggagcaagaaaacaccatgctgcggagagagatcgggaagctgacagaggagctgaagcacctgacagaggcactgaaggagcacgagaagatgtgcccgctgctgctctgccctatgaactttgtgccagtgcctccccggccggaccctgtggccggctgcttgccccgatga

SEQ ID NO: 15 - DNA, 1287 bp, Homo sapiens
  misc_feature: Human IRF8 (DNA sequence)
atgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtcggctccggc

SEQ ID NO: 16 - DNA, 819 bp, Homo sapiens
  misc_feature: Human PU.1 (DNA sequence)
atgttacaggcgtgcaaaatggaagggtttcccctcgtcccccctccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccacggcagcggc

SEQ ID NO: 17 - DNA, 720 bp, Homo sapiens
  misc_feature: Human CCAAT/enhancer-binding protein alpha (CEBPA) (DNA sequence)
atgcccgggggagcgcacgggcccccgcccggctacggctgcgcggccgccggctacctggacggcaggctggagcccctgtacgagcgcgtcggggcgccggcgctgcggccgctggtgatcaagcaggagccccgcgaggaggatgaagccaagcagctggcgctggccggcctcttcccttaccagccgccgccgccgccgccgccctcgcacccgcacccgcacccgccgcccgcgcacctggccgccccgcacctgcagttccagatcgcgcactgcggccagaccaccatgcacctgcagcccggtcaccccacgccgccgcccacgcccgtgcccagcccgcaccccgcgcccgcgctcggtgccgccggcctgccgggccctggcagcgcgctcaaggggctgggcgccgcgcaccccgacctccgcgcgagtggcggcagcggcgcgggcaaggccaagaagtcggtggacaagaacagcaacgagtaccgggtgcggcgcgagcgcaacaacatcgcggtgcgcaagagccgcgacaaggccaagcagcgcaacgtggagacgcagcagaaggtgctggagctgaccagtgacaatgaccgcctgcgcaagcgggtggaacagctgagccgcgaactggacacgctgcggggcatcttccgccagctgccagagagctccttggtcaaggccatgggcaactgcgcgtga

SEQ ID NO: 18 - DNA, 913 bp, Homo sapiens
  misc_feature: Homo sapiens basic leucine zipper ATF-like transcription factor (BATF)
aaagcgagcgacatgtccctttggggagcagtccctctgcaccccagagtgaggaggacgcaggggtcagaggtggctacagggcaggcagaggaggcacctgtagggggtggtgggctggtggcccaggagaagtcaggaagggagcccagctggtgacaagagagcccagaggtgcctggggctgagtgtgagagcccggaagatttcagccatgcctcacagctccgacagcagtgactccagcttcagccgctctcctccccctggcaaacaggactcatctgatgatgtgagaagagttcagaggagggagaaaaatcgtattgccgcccagaagagccgacagaggcagacacagaaggccgacaccctgcacctggagagcgaagacctggagaaacagaacgcggctctacgcaaggagatcaagcagctcacagaggaactgaagtacttcacgtcggtgctgaacagccacgagcccctgtgctcggtgctggccgccagcacgccctcgccccccgaggtggtgtacagcgcccacgcattccaccaacctcatgtcagctccccgcgcttccagccctgagcttccgatgcggggagagcagagcctcgggaggggcacacagactgtggcagagctgcgcccatcccgcagaggcccctgtccacctggagacccggagacagaggcctggacaaggagtgaacacgggaactgtcacgactggaagggcgtgaggcctcccagcagtgccgcagcgtttcgaggggcgtgtgctggaccccaccactgtgggttgcaggcccaatgcagaagagtattaagaaagatgctcaagtcccatggcacagagcaaggcgggcagggaacggttatttttctaaataaatgctttaaaagaaa

SEQ ID NO: 19 - protein, 125 aa, Homo sapiens
  MISC_FEATURE: basic leucine zipper transcriptional factor ATF-like [Homo sapiens], peptide sequence
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 20 - DNA, 2008 bp, Homo sapiens
  misc_feature: Homo sapiens interferon regulatory factor 7 (IRF7)
gagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 21 - protein, 516 aa, Homo sapiens
  MISC_FEATURE: Interferon regulatory factor 7 [Homo sapiens], peptide sequence
MPVPERPAAGPDSPRPGTRRAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 22 - DNA, 3792 bp, Homo sapiens
  misc_feature: Homo sapiens Spi-B transcription factor (SPIB)
ggcaaacagcccgcccggcaccaccatgctcgccctggaggctgcacagctcgacgggccacacttcagctgtctgtacccagatggcgtcttctatgacctggacagctgcaagcattccagctaccctgattcagagggggctcctgactccctgtgggactggactgtggccccacctgtcccagccaccccctatgaagccttcgacccggcagcagccgcttttagccacccccaggctgcccagctctgctacgaaccccccacctacagccctgcagggaacctcgaactggcccccagcctggaggccccggggcctggcctccctgcataccccacggagaacttcgctagccagaccctggttcccccggcatatgccccgtaccccagccctgtgctatcagaggaggaagacttaccgttggacagccctgccctggaggtctcggacagcgagtcggatgaggccctcgtggctggccccgaggggaagggatccgaggcagggactcgcaagaagctgcgcctgtaccagttcctgctggggctactgacgcgcggggacatgcgtgagtgcgtgtggtgggtggagccaggcgccggcgtcttccagttctcctccaagcacaaggaactcctggcgcgccgctggggccagcagaaggggaaccgcaagcgcatgacctaccagaagctggcgcgcgccctccgaaactacgccaagaccggcgagatccgcaaggtcaagcgcaagctcacctaccagttcgacagcgcgctgctgcctgcagtccgccgggcctgagcacacccgaggctcccacctgcggagccgctgggggacctcacgtcccagccaggatccccctggaagaaaaagggcgtccccacactctaggtgataggacttacgcatccccaccttttggggtaaggggagtgctgccctgccataatccccaagcccagcccgggcctgtctgggattccccacttgtgcctggggtcctctgggatttctttgtcatgtacagactccctgggatcctcatgttttgggtgacaggacctatggaccactatactcggggaggcagggtagcagttcttccagaatcccaagagcttctctgggattttcttgtgatatctgattccccagtgaggcctgggacgtttttaagatcgctgtgtgtctgtaaaccctgaatctcatctggggtgggggccctgctggcaaccctgagccctgtccaaggttccctcttgtcagatctgagatttcctagttatgtctggggccctctgggagctgttatcatctcagatctcttcgcccatctatggctgtgttgtcacatctgtcccctcatttttgagatcccccaattctctggaactattctgctgcccctttttatgtgtctggagttccccaatcacatctagggctcctccaagatccttttgtcatgtctgaaatcactcttgagaggtctggggtggaggatggggagtcagtgaaatgtgtcatgtctgggccctgtcagggacacccttgttatatctgggatcctccaatcacatctgagacctcctaggctctccatctgatatgccctttcagggaccccacaaagactgagttctcatggggatcctacccttcctagtgccactccctatggccatgctgaagaccactctggccacgcgactgattttgggtgatcatggcagctccccacccatgtcatttctaaccagaagtctcaaggtcgtcacccccctgccccccaaccgaggccccggtcgctggtggtggtctctttagtgcactgtagcacttggtggtggaggtgtgagggatccacattaacagcaggccatcagctgggcaatggctcacacctgtaatcccagcactttgggaggcgaggcagggggaatggcttgaacccaggcattcaagaccagcctgggcaacataatgagacctcgtctctacaaaacataacaaaaacaattagccgagcgtgggggtgaacacctgtggtcccagctgctcaggaggctgaggtgggaggatctcttgagcccaggaagtaggaggctgtagtgagctgtaatcgtgccactgcactccagcctgggcgacagagtgagacaccgtcttaaaaacaaaaacaaggccgggcacggtggctcatgcctgttgtcccagcactttgggaggccgaggcaggcggatcacgaggtcgagagatcgagaccatcctggccaacatggtgaaaccctgtctctactaaaaatacagaaattagctgggcgtggtggcacgtgcctgtagtcccagctactcgggaggctgaggcaagagaatcgcttgaacgtgggaggcagaggttgcagtgagcctagattgtgccactgcactccagcctgggggacagagcgagactccgtctgaaaataaaaacaacaaaaacagcagaccattcaaaatagggagactttgcataatccagatttctgccttcacttaaaactttggacggtctggagagagtcggccagttttcggtggggggtggggagctggaacaggacagtagcctttcctaatgaggcatttgttctccaatctgccccagtcgctgccatccctggctatctcaccctagcagcttctcaagcctgttggctttagaccactgtataaacccagctggaactgaagcctgggtggactatggagccctggttgggacccccagggagtcaaaggctgcgggccaagaggccagaggtccttgagcctgggtgggcaggtggatctagggtgcatgacttgctgcttcccaaccttagtttgtcccttctgtgaaaaagggagagaaggaggaggaagatctcaaaaagactttccagcccagtgcggtggctcacgcctgtaatcccagcactttgggaggccgatgcaggtggatcacctgaggtaggagttcaagaccagcctgaccaacatagtgaagccccttctctactaaaaatacaaaattagctgggcgtggtggcatgtgcctgtactcccagctacttgggaggctgaggcaggagaatcgcttgaacctgggaggcggaggttgtagtgagctgagatcacaccactgcacaccagcctgggcgacaagagcgaaactccgtctcaaaaaaaaaaaactgttgcagccccgttgagcctttgacaccgcctgaaatccaccccactcccaggaggaggaggaggaaggaatgccaatgacctagagacacgagaagtccatgtggaggcacacagcagctgatggcagagcccaggctgggacctgcccttaagagaatgagtgggaagggggagggaggaagggcaggtaaaacgtcctccccagggccccctgcaacggggaaggtactttttacaaaagctatcattgtcaccctaaatgtggaataaaataagatgcatcgacgtagacaaacctcctgggaccttttgtcagggactgcaatcctgcccctccactgaggccgctggctctcagagacaccgtgacatcacgggtgatgatgagaggagttcaaagagagaattatatgctggcgcggtggctctgtaatcccaacactttggggggccaaggcaggaggatcgcttgagtacaggagtttgaaaccagcctgggcaagatagtgagatccccttcccacccgtctacaaaaaaaataaaaaattagcgggg

SEQ ID NO: 23 - protein, 262 aa, Homo sapiens
  MISC_FEATURE: transcription factor Spi-B [Homo sapiens], peptide sequence
MLALEAAQLDGPHFSCLYPDGVFYDLDSCKHSSYPDSEGAPDSLWDWTVAPPVPATPYEAFDPAAAAFSHPQAAQLCYEPPTYSPAGNLELAPSLEAPGPGLPAYPTENFASQTLVPPAYAPYPSPVLSEEEDLPLDSPALEVSDSESDEALVAGPEGKGSEAGTRKKLRLYQFLLGLLTRGDMRECVWWVEPGAGVFQFSSKHKELLARRWGQQKGNRKRMTYQKLARALRNYAKTGEIRKVKRKLTYQFDSALLPAVRRA

SEQ ID NO: 24 - DNA, 1217 bp, Homo sapiens
  misc_feature: Homo sapiens Spi-C transcription factor (SPIC)
gatggttatgtcttaacaattctaatttttaaaaaaaatattactattttttttcatcagagccattgcactggaaaataattttttattttcatgatagcctaccgttgaatttaccgttctgatattaatgaaacatctctataaagggttgaagtgtcttcccggattgtcaacttattttattttattttcttcaagcaacaattgctaaggaacagaattgtcaatttattaatgaaatatgacgtgtgttgaacaagacaagctgggtcaagcatttgaagatgcttttgaggttctgaggcaacattcaactggagatcttcagtactcgccagattacagaaattacctggctttaatcaaccatcgtcctcatgtcaaaggaaattccagctgctatggagtgttgcctacagaggagcctgtctataattggagaacggtaattaacagtgctgcggacttctattttgaaggaaatattcatcaatctctgcagaacataactgaaaaccagctggtacaacccactcttctccagcaaaaggggggaaaaggcaggaagaagctccgactgtttgaataccttcacgaatccctgtataatccggagatggcatcttgtattcagtgggtagataaaaccaaaggcatctttcagtttgtatcaaaaaacaaagaaaaacttgccgagctttgggggaaaagaaaaggcaacaggaagaccatgacttaccagaaaatggccagggcactcagaaattacggaagaagtggggaaattaccaaaatccggaggaagctgacttaccagttcagtgaggccattctccaaagactctctccatcctatttcctggggaaagagatcttctattcacagtgtgttcaacctgatcaagaatatctcagtttaaataactggaatgcaaattataattatacatatgccaattaccatgagctaaatcaccatgattgctaaatatactttcatatttcatggtttactggcatcggaaatctctacaagttttaatgatttctccctccctctctttttttcctcctctgaagaaatttaggatttttctcttaaagcaaatactaaagaggaaaaaaaattaactttattgttgcttttatcaaagagtatgtaatctatactaacttgttgggaaattctgccaatgaacaacttttttataata

SEQ ID NO: 25 - protein, 248 aa, Homo sapiens
  MISC_FEATURE: transcription factor Spi-C [Homo sapiens], peptide sequence
MTCVEQDKLGQAFEDAFEVLRQHSTGDLQYSPDYRNYLALINHRPHVKGNSSCYGVLPTEEPVYNWRTVINSAADFYFEGNIHQSLQNITENQLVQPTLLQQKGGKGRKKLRLFEYLHESLYNPEMASCIQWVDKTKGIFQFVSKNKEKLAELWGKRKGNRKTMTYQKMARALRNYGRSGEITKIRRKLTYQFSEAILQRLSPSYFLGKEIFYSQCVQPDQEYLSLNNWNANYNYTYANYHELNHHDC